(19) United States
(12) Patent Application Publication
(10) Pub. No.: US 2009/0062294 A1
(43) Pub. Date: Mar. 5, 2009

(54) HETEROARYL-SUBSTITUTED UREA MODULATORS OF FATTY ACID AMIDE HYDROLASE

(76) Inventors: Richard Apodaca, San Diego, CA (US); J. Guy Breitenbucher, Escondido, CA (US); Natalie A. Hawryluk, San Diego, CA (US); William M. Jones, San Diego, CA (US); John M. Keith, San Diego, CA (US); Jeffrey E. Merit, San Diego, CA (US); Mark S. Tichenor, San Diego, CA (US); Amy K. Timmons, Renton, WA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/126,389

(22) Filed: May 23, 2008

Related U.S. Application Data
(60) Provisional application No. 60/931,920, filed on May 25, 2007.

(51) Int. Cl. A61K 31/501; C07D 401/00; C07D 403/00; C07D 403/14; A61P 35/00; A61P 25/00; A61P 1/04; A61P 9/00; A61P 37/00; A61P 31/12; A61P 1/00; A61K 31/445; A61K 31/496

(52) U.S. Cl. 514/252.02; 544/368; 514/254.04; 514/254.03; 514/254.05; 544/366; 544/379; 514/252.13; 514/252.03; 544/238; 514/326; 546/209; 544/371; 514/252.11; 514/252.14; 544/316; 544/363; 514/252.18

(57) ABSTRACT

Certain heteroaryl-substituted piperidinyl and piperazinyl urea compounds are described, which are useful as FAAH inhibitors. Such compounds may be used in pharmaceutical compositions and methods for the treatment of disease states, disorders, and conditions mediated by fatty acid amide hydrolase (FAAH) activity, such as anxiety, pain, inflammation, sleep disorders, eating disorders, insulin resistance, diabetes, osteoporosis, and movement disorders (e.g., multiple sclerosis).

This application claims the benefit of U.S. provisional patent application Ser. No. 60/931,920, filed on May 25, 2007, which is incorporated herein by reference.

### FIELD OF THE INVENTION

The present invention relates to certain heteroaryl-substituted piperidinyl and piperazinyl urea compounds, pharmaceutical compositions containing them, and methods of using them for the treatment of disease states, disorders, and conditions mediated by fatty acid amide hydrolase (FAAH) activity.

### BACKGROUND OF THE INVENTION

Medicinal benefits have been attributed to the cannabis plant for centuries. The primary bioactive constituent of cannabis is Δ⁹-tetrahydro-cannabinol (THC). The discovery of THC eventually led to the identification of two endogenous cannabinoid receptors responsible for its pharmacological actions, namely CB₁ and CB₂ (Goya, Exp. Opin. Ther. Patents 2000, 10, 1529). These discoveries not only established the site of action of THC, but also inspired inquiries into the endogenous agonists of these receptors, or “endocannabinoids”. The first endocannabinoid identified was the fatty acid amide anandamide (AEA). AEA itself elicits many of the pharmacological effects of exogenous cannabinoids (Piomelli, Nat. Rev. Neurosci. 2003, 4(11), 873).

The catabolism of AEA is primarily attributable to the integral membrane bound protein fatty acid amide hydrolase (FAAH), which hydrolyzes AEA to arachidonic acid. FAAH was characterized in 1996 by Cravatt and co-workers (Cravatt, Nature 1996, 384, 83). It was subsequently determined that FAAH is additionally responsible for the catabolism of a large number of important lipid signaling fatty acid amides including: another major endocannabinoid, 2-arachidonoylglycerol (2-AG) (Science 1992, 258, 1946-1949); the sleep-inducing substance, oleamide (OEA) (Science 1995, 268, 1506); the appetite-suppressing agent, N-oleoylethanolamine (Rodriguez de Fonesca, Nature 2001, 414, 209); and the anti-inflammatory agent, palmitoylethanolamide (PEA) (Lambert, Curr. Med. Chem. 2002, 9(6), 663).

Small-molecule inhibitors of FAAH should elevate the concentrations of these endogenous signaling lipids and thereby produce their associated beneficial pharmacological effects. There have been some reports of the effects of various FAAH inhibitors in pre-clinical models.

In particular, two carbamate-based inhibitors of FAAH were reported to have analgesic properties in animal models. In rats, BMS-1 (see WO 02/087569), which has the structure shown below, was reported to have an analgesic effect in the Chung spinal nerve ligation model of neuropathic pain, and the Hargraves test of acute thermal nociception. URB-597 was reported to have efficacy in the zero plus maze model of anxiety in rats, as well as analgesic efficacy in the rat hot plate and formalin tests (Kathuria, Nat. Med. 2003, 9(1), 76). The sulfonylfluoride AM374 was also shown to significantly reduce spasticity in chronic relapsing experimental autoimmune encephalomyelitis (CREAE) mice, an animal model of multiple sclerosis (Baker, FASEB J. 2001, 15(2), 300).

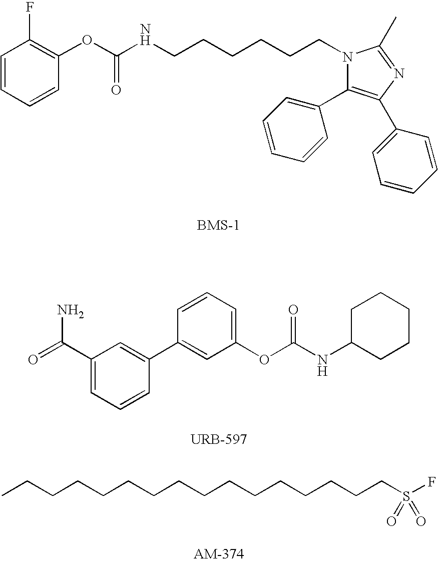

In addition, the oxazolopyridine ketone OL-135 is reported to be a potent inhibitor of FAAH, and has been reported to have analgesic activity in both the hot plate and tail emersion tests of thermal nociception in rats (WO 04/033652).

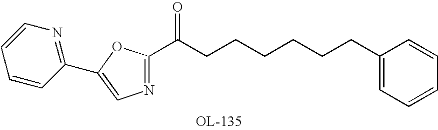

Results of research on the effects of certain exogenous cannabinoids has elucidated that a FAAH inhibitor may be useful for treating various conditions, diseases, disorders, or symptoms. These include pain, nausea/emesis, anorexia, spasticity, movement disorders, epilepsy and glaucoma. To date, approved therapeutic uses for cannabinoids include the relief of chemotherapy-induced nausea and emesis among patients with cancer and appetite enhancement in patients with HIV/AIDs who experience anorexia as a result of wasting syndrome. Two products are commercially available in some countries for these indications, namely, dronabinol (Marinol®) and nabilone.

Apart from the approved indications, a therapeutic field that has received much attention for cannabinoid use is analgesia, i.e., the treatment of pain. Five small randomized controlled trials showed that THC is superior to placebo, producing dose-related analgesia (Robson, Br. J. Psychiatry 2001, 178, 107-115). Atlantic Pharmaceuticals is reported to be developing a synthetic cannabinoid, CT-3, a 1,1-dimethyl heptyl derivative of the carboxylic metabolite of tetrahydrocannabinol, as an orally active analgesic and anti-inflammatory agent. A pilot phase II trial in chronic neuropathic pain with CT-3 was reportedly initiated in Germany in May 2002.

A number of individuals with locomotor activity-related diseases, such as multiple sclerosis have claimed a benefit from cannabis for both disease-related pain and spasticity, with support from small controlled trials (Croxford et el., J. Neuroimmunol, 2008, 193, 120-9; Svendsen, Br. Med. J. 2004, 329, 253). Likewise, various victims of spinal cord injuries, such as paraplegia, have reported that their painful spasms are alleviated after smoking marijuana. A report showing that cannabinoids appear to control spasticity and tremor in the CREAE model of multiple sclerosis demonstrated that these effects are mediated by CB₁ and CB₂ receptors (Baker, Nature 2000, 404, 84-87). Phase 3 clinical trials have been undertaken in multiple sclerosis and spinal cord injury patients with a narrow ratio mixture of tetrahydrocannabinol/cannabidiol (THC/CBD).

Reports of small-scale controlled trials to investigate other potential commercial uses of cannabinoids have been made. Trials in volunteers have been reported to have confirmed that oral, injected, and smoked cannabinoids produced dose-related reductions in intraocular pressure (IOP) and therefore may relieve glaucoma symptoms. Ophthalmologists have prescribed cannabis for patients with glaucoma in whom other drugs have failed to adequately control intraocular pressure (Robson, 2001, supra).

Inhibition of FAAH using a small-molecule inhibitor may be advantageous compared to treatment with a direct-acting CB₁ agonist. Administration of exogenous CB₁ agonists may produce a range of responses, including reduced nociception, catalepsy, hypothermia, and increased feeding behavior. These four in particular are termed the “cannabinoid tetrad.” Experiments with FAAH −/− mice show reduced responses in tests of nociception, but did not show catalepsy, hypothermia, or increased feeding behavior (Cravatt, Proc. Natl. Acad. Sci. USA 2001, 98(16), 9371). Fasting caused levels of AEA to increase in rat limbic forebrain, but not in other brain areas, providing evidence that stimulation of AEA biosynthesis may be anatomically regionalized to targeted CNS pathways (Kirkham, Br. J. Pharmacol. 2002, 136, 550). The finding that AEA increases are localized within the brain, rather than systemic, suggests that FAAH inhibition with a small molecule could enhance the actions of AEA and other fatty acid amides in tissue regions where synthesis and release of these signaling molecules is occurring in a given pathophysiological condition (Piomelli, 2003, supra).

In addition to the effects of a FAAH inhibitor on AEA and other endocannabinoids, inhibitors of FAAH's catabolism of other lipid mediators may be used in treating certain other therapeutic indications. For example, PEA has demonstrated biological effects in animal models of inflammation (Holt, et al. Br. J. Pharmacol. 2005, 146, 467-476), immunosuppression, analgesia, and neuroprotection (Ueda, J. Biol. Chem. 2001, 276(38), 35552). Oleamide, another substrate of FAAH, induces sleep (Boger, Proc. Natl. Acad. Sci. USA 2000, 97(10), 5044; Mendelson, Neuropsychopharmacology 2001, 25, S36). Inhibition of FAAH has also been implicated in cognition (Varvel et al., J. Pharmacol. Exp. Ther. 2006, 317(1), 251-257) and depression (Gobbi et al., Proc. Natl. Acad. Sci. USA 2005, 102(51), 18620-18625).

Two additional indications for FAAH are supported by recent data indicating that FAAH substrate activated receptors are important in energy metabolism, and in bone homeostasis (Overton et al., Br. J. Pharmacol. 2008, in press; and Plutzky, Diab. Vasc. Dis. Res. 2007, 4 Suppl 3, S12-4). It has been shown that the previously mentioned lipid signaling fatty acid amides catabolized by FAAH, oleoylethanolamide (OEA), is one of the most active agonists of the recently de-orphanised GPCR 119 (GPR119) (also termed glucose dependent insulinotropic receptor). This receptor is expressed predominantly in the pancreas in humans and activation improves glucose homeostasis via glucose-dependent insulin release in pancreatic beta-cells. GPR119 agonists can suppress glucose excursions when administered during oral glucose tolerance tests, and OEA has also been shown independently to regulate food intake and body weight gain when administered to rodents, indicating a probable benefit in energy metabolism disorders, such as insulin resistance and diabetes. The FAAH substrate palmitoylethanolamide (PEA) is an agonist at the PPARα receptor. Evidence from surrogate markers in human studies with the PPARα agonist fenofibrate is supportive of the concept that PPARα agonism offers the potential for inducing a coordinated PPARα response that may improve dyslipidaemia, repress inflammation and limit atherosclerosis in patients with the metabolic syndrome or type 2 diabetes. The FAAH substrate anandamide (AEA) is an agonist at the PPARγ receptor. Anandamide treatment induces 3T3-L1 differentiation into adipocytes, as well as triglyceride droplet accumulation and expression of adiponectin (Bouaboula et al., E. J. Pharmacol. 2005, 517, 174-181). Low dose cannabinoid therapy has been shown to reduce atherosclerosis in mice, further suggesting a therapeutic benefit of FAAH inhibition in dyslipidemia, liver steatosis, steatohepatitis, obesity, and metabolic syndrome (Steffens et al., Nature, 2005, 434, 782-6).

Osteoporosis is one of the most common degenerative diseases. It is characterized by reduced bone mineral density (BMD) with an increased risk for bone fractures. CB₂-deficient mice have a markedly accelerated age-related trabecular bone loss and cortical expansion. A CB₂-selective agonism enhances endocortical osteoblast number and activity and restrains trabecular osteoclastogenesis and attenuates ovariectomy-induced bone loss (Ofek et al., Proc. Natl. Acad. Sci. U.S.A. 2006, 103, 696-701). There is a substantial genetic contribution to BMD, although the genetic factors involved in the pathogenesis of human osteoporosis are largely unknown. The applicability to human BMD is suggested by genetic studies in which a significant association of single polymorphisms and haplotypes was found encompassing the CNR2 gene on human chromosome 1p36, demonstrating a role for the peripherally expressed CB₂ receptor in the etiology of osteoporosis (Karsak et al., Hum. Mol. Genet, 2005, 14, 3389-96).

Thus, small-molecule FAAH inhibitors should be useful in treating pain of various etiologies, anxiety, multiple sclerosis and other movement disorders, nausea/emesis, eating disorders, epilepsy, glaucoma, inflammation, immunosuppression, neuroprotection, depression, cognition enhancement, and sleep disorders, and potentially with fewer side effects than treatment with an exogenous cannabinoid.

A number of heteroaryl-substituted ureas have been reported in various publications. Certain substituted thiophene ureas are described in U.S. Pat. No. 6,881,741. Certain ureido-pyrazoles are described in U.S. Pat. No. 6,387,900. Certain benzothiazole amide derivatives are described in US Patent Publication US 2003/149036. Certain ureas are reported as prenyltransferase inhibitors in WO 2003/047569. Piperidinyl ureas are described as histamine H₃ receptor antagonists in U.S. Pat. No. 6,100,279. Piperazinyl ureas are disclosed as calcitonin mimetics in U.S. Pat. Nos. 6,124,299 and 6,395,740. Various ureas are reported as small-molecule FAAH modulators in US Patent Publication Nos. US 2006/173184 and US 2007/0004741, in Intl. Patent Appl. Nos. WO 2008/023720, WO 2008/047229, and WO 2008/024139, and by Cravatt et al. (Biochemistry 2007, 46(45), 13019. Ureas are described as modulators of other targets in U.S. Pat. Appl. Publ. US 2007/270433, and in Intl. Pat. Appl. Publ. Nos. WO 2007/096251 and WO 2006/085108. However, there remains a desire for potent FAAH modulators with suitable pharmaceutical properties.

### SUMMARY OF THE INVENTION

Certain heteroaryl-substituted piperidinyl and piperazinyl urea derivatives have now been found to have FAAH-modulating activity. Thus, the invention is directed to the general and preferred embodiments defined, respectively, by the independent and dependent claims appended hereto, which are incorporated by reference herein.

In one general aspect, the invention is directed to compounds of Formula (I):

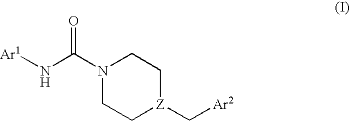

wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, 1H-pyrazol-3-yl, 5-methylpyrazin-2-yl, 3-chloropyrazin-2-yl, pyridazin-3-yl, 6-methoxypyridazin-3-yl, 5-methyl isoxazol-3-yl, 1,5-dimethyl-1H-pyrazol-3-yl, 4-bromo-1-methyl-1H-pyrazol-3-yl, 2-ethyl-2H-pyrazol-3-yl, 5-methyl-1H-pyrazol-3-yl, or 5-phenyl-1H-pyrazol-3-yl group;
Z is —N— or >CH; and
Ar² is:
   (i) phenyl unsubstituted or substituted with one or two R^{a} moieties;
      where each R^{a} moiety is independently —C₁₋₄alkyl, —C≡C—R^{d}, —OC₁₋₄alkyl, halo, —CF₃, —OCF₃, —OCH₂CF₃, —SCF₃, —S(O)₀₋₂C₁₋₄alkyl, —SO₂CF₃, —OSO₂C₁₋₄alkyl, —(CH₂)₀₋₁CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —(CH₂)₀₋₁CN;
         or two adjacent R^{a} moieties taken together form —O(CH₂)₁₋₂O— or —OCF₂O—;
         where R^{b} and R^{c} are each independently —H or —C₁₋₄alkyl; and
         R^{d} is H, C₃₋₆cycloalkyl, or —CH₂NR^{e}R^{f};
            where R^{e} and R^{f} are each independently H or C₁₋₄alkyl;
   (ii) phenyl substituted at the 3- or 4-position with -L-Ar³, unsubstituted or substituted with one or two R^{a} moieties, wherein:
      L is a linker selected from the group consisting of —(CH₂)₁₋₃—, —CH═CH—, —O—, —OCH₂—, —CH₂O—, —NH—, >NC₁₋₄alkyl, —S—, —C≡C—, —C(═O)—, and
         a covalent bond; and
      Ar³ is:
         (a) phenyl;
         (b) naphthyl; or
         (c) a monocyclic or bicyclic heteroaryl group; or
   (iii) a 9- or 10-membered fused bicyclic heteroaryl group;
      where when Ar¹ is 6-chloro-pyridazin-3-yl, isoxazol-3-yl, or 1H-pyrazol-3-yl,
   then Ar² is not benzo[1,3]dioxol-5-yl or 2,2-difluoro-benzo[1,3]dioxol-5-yl; and pharmaceutically acceptable salts, pharmaceutically acceptable prodrugs, and pharmaceutically active metabolites of said compounds.

In another general aspect, the invention is directed to compounds of Formula (Ia):

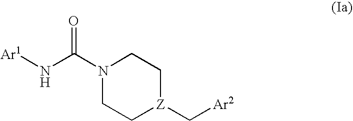

wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, or 1H-pyrazol-3-yl group;
Z is —N— or >CH; and
Ar² is:
   (i) phenyl or 3-phenoxyphenyl substituted with one or two R^{a} moieties;
      where each R^{a} moiety is independently —C₁₋₄alkyl, —OC₁₋₄alkyl, halo, —CF₃, —OCF₃, —OCH₂CF₃, —SCF₃, —S(O)₀₋₂C₁₋₄alkyl, —OSO₂C₁₋₄alkyl, —CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —CN;
         where R^{b} and R^{c} are each independently —H or —C₁₋₄alkyl; or
   (ii) benzo[1,3]dioxol-5-yl, 2,2-difluoro-benzo[1,3]dioxol-5-yl, or naphthyl;
      where when Ar¹ is 6-chloro-pyridazin-3-yl, isoxazol-3-yl, or 1H-pyrazol-3-yl,

then Ar² is not benzo[1,3]dioxol-5-yl or 2,2-difluoro-benzo[1,3]dioxol-5-yl; and pharmaceutically acceptable salts, pharmaceutically acceptable prodrugs, and pharmaceutically active metabolites of such compounds. In especially preferred embodiments, the invention is directed to compounds described or exemplified in the detailed description below and their pharmaceutically acceptable salts.

One skilled in the art will recognize that compounds of Formula (Ia) are embodiments of compounds of Formula (I). References herein to compounds of Formula (I) therefore encompass compounds of Formula (Ia) as well.

In a further general aspect, the invention relates to pharmaceutical compositions each comprising: (a) an effective amount of at least one agent selected from compounds of Formula (I), pharmaceutically acceptable salts of compounds of Formula (I), pharmaceutically acceptable prodrugs of compounds of Formula (I), and pharmaceutically active metabolites of Formula (I); and (b) a pharmaceutically acceptable excipient.

In another general aspect, the invention is directed to a method of treating a subject suffering from or diagnosed with a disease, disorder, or medical condition mediated by FAAH activity, comprising administering to the subject in need of such treatment an effective amount of at least one agent selected from compounds of Formula (I) and their pharmaceutically acceptable salts, pharmaceutically active prodrugs, and pharmaceutically active metabolites. In preferred embodiments of the inventive method, the disease, disorder, or medical condition is selected from: anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, symptoms of drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, auto-immune diabetes, intractable pruritis, and neuroinflammation.

Additional embodiments, features, and advantages of the invention will be apparent from the following detailed description and through practice of the invention.

### DETAILED DESCRIPTION OF INVENTION AND ITS PREFERRED EMBODIMENTS

The invention may be more fully appreciated by reference to the following detailed description, including the following glossary of terms and the concluding examples. For the sake of brevity, the disclosures of the publications, including patents, cited in this specification are herein incorporated by reference.

As used herein, the terms “including”, “containing” and “comprising” are used in their open, non-limiting sense.

The term “alkyl” refers to a straight- or branched-chain alkyl group having from 1 to 12 carbon atoms in the chain. Examples of alkyl groups include methyl (Me, which also may be structurally depicted by / symbol), ethyl (Et), n-propyl, isopropyl, butyl, isobutyl, sec-butyl, tert-butyl (tBu), pentyl, isopentyl, tert-pentyl, hexyl, isohexyl, and so on.

The term “alkenyl” refers to a straight- or branched-chain alkenyl group having from 2 to 12 carbon atoms in the chain. (The double bond of the alkenyl group is formed by two sp² hybridized carbon atoms.) Illustrative alkenyl groups include prop-2-enyl, but-2-enyl, but-3-enyl, 2-methylprop-2-enyl, hex-2-enyl, and so on.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

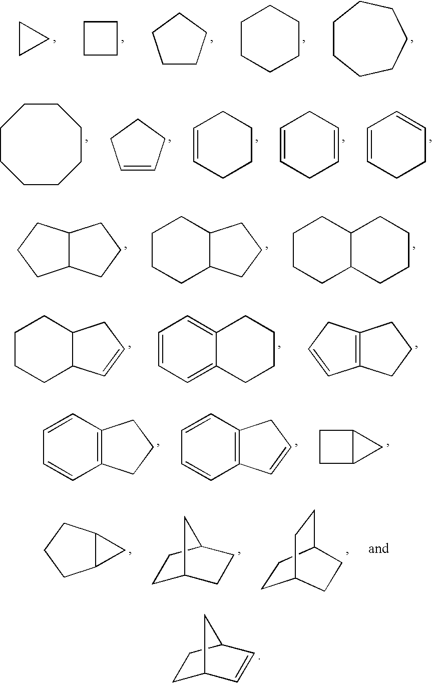

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative examples of heterocycloalkyl groups include the following entities, in the form of properly bonded moieties:

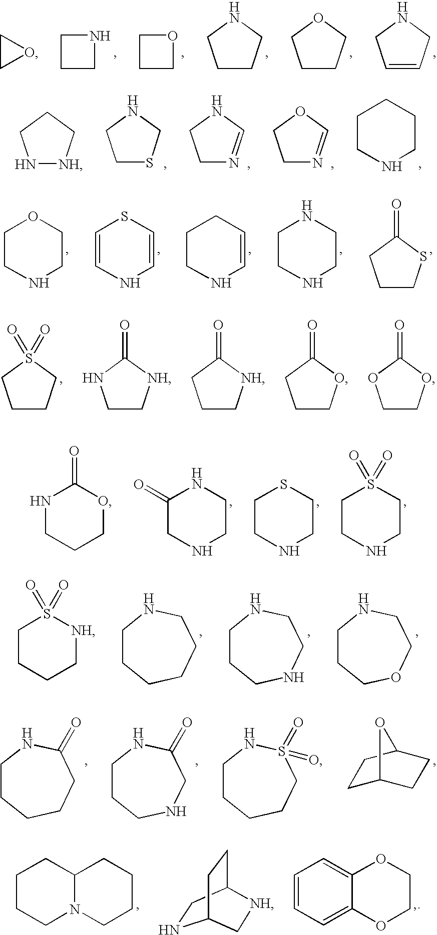

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

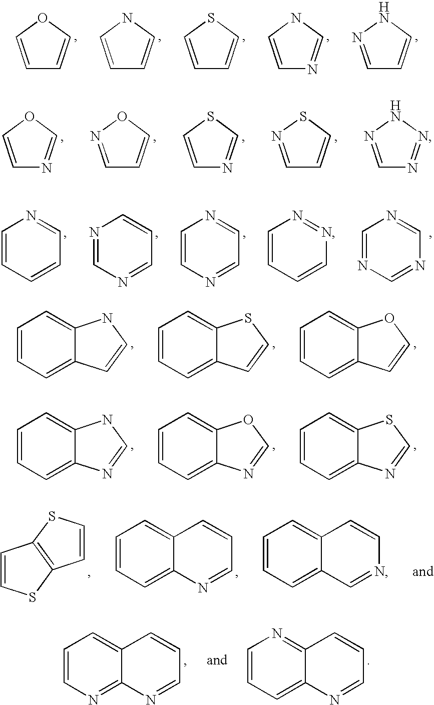

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

A structural formula given herein is intended to represent compounds having structures depicted by the formula as well as equivalent variations or forms. For example, compounds encompassed by Formula (I) may have asymmetric centers and therefore exist in different enantiomeric forms. All optical isomers and stereoisomers of the compounds of the general formula, and mixtures thereof, are considered within the scope of the formula. Thus, a general formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers (e.g. pyrazole, benzimidazole, tetrazole, or benzotriazole tautomers), or as atropisomers, which are intended to be represented by the structural formula. Additionally, a formula given herein is intended to embrace hydrates, solvates, and polymorphs of such compounds, and mixtures thereof.

A structural formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸O, ¹⁷O, ³²P, ³³P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)], including drug or substrate tissue distribution assays, or in radioactive treatment of patients. In particular, an ¹⁸F— or ¹¹C-labeled compound may be preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a formula variable appears more than once, the choice of the species from a specified list is independent of the choice of the species for the same variable elsewhere in the formula.

In preferred embodiments of Formula (I), Ar¹ is a benzo[d]isoxazol-3-yl, 6-fluorobenzo[d]isoxazol-3-yl, benzo[1,2,5]thiadiazol-4-yl, benzo[1,2,5]oxadiazol-4-yl, 6-chloro-pyridazin-3-yl, pyrazin-2-yl, isoxazol-3-yl, 1H-benzotriazol-5-yl, benzothiazol-6-yl, or 1H-pyrazol-3-yl group. In further preferred embodiments, Ar¹ is a benzo[d]isoxazol-3-yl group. In still further preferred embodiments, Ar¹ is a pyrazin-2-yl group. In still further preferred embodiments, Ar¹ is an isoxazol-3-yl group. In still further preferred embodiments, Ar¹ is a pyridazin-3-yl group.

In preferred embodiments, Z is —N—. In other preferred embodiments, Z is >CH.

In preferred embodiments, Ar² is phenyl, substituted with one or two R^{a} moieties.

In preferred embodiments, Ar² is phenyl, substituted with one or two R^{a} moieties, and each R^{a} moiety is independently selected from the group consisting of: chloro, cyano, isobutyl, methylsulfanyl, methanesulfonyl, trifluoromethyl, trifluoromethoxy, 2,2,2-trifluoroethoxy, fluoro, methyl, methoxy, tert-butyl, bromo, methoxycarbonyl, cyanomethyl, methoxycarbonylmethyl, trifluoromethanesulfonyl, trifluoromethanesulfanyl, and butyl; or two adjacent R^{a} moieties taken together form —OCH₂O— or —OCF₂O—.

In further preferred embodiments, Ar² is phenyl substituted at the 3- or 4-position with -L-Ar³, to form a -phenyl-L-Ar³ group that is unsubstituted or substituted with one or two R^{a} moieties. In further preferred embodiments, L is —CH₂CH₂—, —O—, —OCH₂—, or —C≡C—. In still further preferred embodiments, Ar³ is phenyl. In still further preferred embodiments, Ar³ is phenyl and each R^{a} moiety is independently selected from the group consisting of: chloro, cyano, isobutyl, methylsulfanyl, methanesulfonyl, trifluoromethyl, trifluoromethoxy, 2,2,2-trifluoroethoxy, fluoro, methyl, methoxy, tert-butyl, bromo, methoxycarbonyl, cyanomethyl, methoxycarbonylmethyl, trifluoromethanesulfonyl, trifluoromethanesulfanyl, and butyl; or two adjacent R^{a} moieties taken together form —OCH₂O— or —OCF₂O—.

In still further preferred embodiments, Ar³ is naphthyl. In still further preferred embodiments, Ar³ is a monocyclic or bicyclic heteroaryl group. In still further preferred embodiments, Ar³ is a thiophenyl, pyrimidinyl, pyridyl, pyrazinyl, or quinolinyl group. In still further preferred embodiments, Ar³ is naphthyl or a monocyclic or bicyclic heteroaryl group and each R^{a} moiety is independently selected from the group consisting of: chloro, cyano, isobutyl, methylsulfanyl, methanesulfonyl, trifluoromethyl, trifluoromethoxy, 2,2,2-trifluoroethoxy, fluoro, methyl, methoxy, tert-butyl, bromo, methoxycarbonyl, cyanomethyl, methoxycarbonyl methyl, trifluoromethanesulfonyl, trifluoromethanesulfanyl, and butyl; or two adjacent R^{a} moieties taken together form —OCH₂O— or —OCF₂O—.

In further preferred embodiments, Ar² is a 9- or 10-membered fused bicyclic heteroaryl group. In still further preferred embodiments, Ar² is a benzimidazolyl, indazolyl, benzothiophenyl, quinolinyl, indolyl, or benzofuranyl group.

In preferred embodiments of Formula (I) or (Ia), Ar¹ is a benzo[d]isoxazol-3-yl, 6-fluorobenzo[d]isoxazol-3-yl, benzo[1,2,5]thiadiazol-4-yl, benzo[1,2,5]oxadiazol-4-yl, 6-chloro-pyridazin-3-yl, pyrazin-2-yl, isoxazol-3-yl, 1H-benzotriazol-5-yl, benzothiazol-6-yl, or 1H-pyrazol-3-yl group. In further preferred embodiments, Ar¹ is a benzo[d]isoxazol-3-yl group. In still further preferred embodiments, Ar¹ is a pyrazin-2-yl group. In still further preferred embodiments, Ar¹ is an isoxazol-3-yl group. In still further preferred embodiments, Ar¹ is a pyridazin-3-yl group.

In preferred embodiments, Ar² is 3-phenoxyphenyl substituted with one or two R^{a} moieties independently selected from the group consisting of fluoro, chloro, bromo, —CF₃, —OCF₃, or —OCH₂CF₃. In other preferred embodiments, Ar² is naphthyl.

The invention also relates to pharmaceutically acceptable salts of the free acids or bases represented by Formula (I), preferably of the preferred embodiments described above and of the specific compounds exemplified herein. The therapeutic compositions and methods of the invention may employ pharmaceutically acceptable salts of the free acids or bases represented by Formula (I), preferably of the preferred embodiments described above and of the specific compounds exemplified herein. A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, by treatment of the free base with an inorganic acid, such as hydrochloric acid, hydrobromic acid, sulfuric acid, sulfamic acid, nitric acid, boric acid, phosphoric acid, and the like; or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid; an amino acid, such as aspartic acid or glutamic acid; an aromatic acid, such as benzoic acid, 2-acetoxybenzoic acid, naphthoic acid, or cinnamic acid; a sulfonic acid, such as laurylsulfonic acid, p-toluenesulfonic acid, methanesulfonic acid, or ethanesulfonic acid; or any compatible mixture of acids such as those given as examples herein.

If the compound of Formula (I) is an acid such as a carboxylic acid or sulfonic acid, the desired pharmaceutically acceptable salt may be prepared by any suitable method, for example, by treatment of the free acid with an inorganic or organic base, such as an amine (primary, secondary or tertiary), an alkali metal hydroxide, alkaline earth metal hydroxide, or any compatible mixture of bases such as those given as examples herein. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I). The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Fleisher et al., Adv. Drug Delivery Rev. 1996, 19, 115-130. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in Robinson et al., J. Med. Chem. 1996, 39, 10-18. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I). A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

The compounds of Formula (I), and their pharmaceutically acceptable salts, pharmaceutically acceptable prodrugs, and pharmaceutically active metabolites (collectively, “active agents”) of the present invention are useful as FAAH inhibitors in the methods of the invention. The active agents may be used in the inventive methods for the treatment of medical conditions, diseases, or disorders mediated through inhibition or modulation of FAAH, such as those described herein. Active agents according to the invention may therefore be used as an analgesic, anti-depressant, cognition enhancer, neuroprotectant, sedative, appetite stimulant, or contraceptive.

Exemplary medical conditions, diseases, and disorders mediated by FAAH activity include anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, epilepsy, Niemann-Pick disease, Parkinson's disease, Huntington's chorea, optic neuritis, autoimmune uveitis, symptoms of drug withdrawal, nausea, emesis, sexual dysfunction, post-traumatic stress disorder, cerebral vasospasm, diabetes, metabolic syndrome and osteoporosis.

Thus, the active agents may be used to treat subjects diagnosed with or suffering from such a disease, disorder, or condition. The term “treat” or “treating” as used herein is intended to refer to administration of an agent or composition of the invention to a subject for the purpose of effecting a therapeutic benefit through modulation of FAAH activity. Treating includes reversing, ameliorating, alleviating, inhibiting the progress of, lessening the severity of, reducing the incidence of, or preventing a disease, disorder, or condition, or one or more symptoms of such disease, disorder or condition mediated through modulation of FAAH activity. The term “subject” refers to a mammalian patient in need of such treatment, such as a human. “Modulators” include both inhibitors and activators, where “inhibitors” refer to compounds that decrease, prevent, inactivate, desensitize or down-regulate FAAH expression or activity, and “activators” are compounds that increase, activate, facilitate, sensitize, or up-regulate FAAH expression or activity.

Accordingly, the invention relates to methods of using the active agents described herein to treat subjects diagnosed with or suffering from a disease, disorder, or condition mediated through FAAH activity, such as: anxiety, pain, sleep disorders, eating disorders, inflammation, movement disorders (e.g., multiple sclerosis), energy metabolism (e.g. insulin resistance, diabetes, dyslipidemia, liver steatosis, steatohepatitis, obesity, and metabolic syndrome) and bone homeostasis (e.g. osteoporosis).

Symptoms or disease states are intended to be included within the scope of “medical conditions, disorders, or diseases.” For example, pain may be associated with various diseases, disorders, or conditions, and may include various etiologies. Illustrative types of pain treatable with a FAAH-modulating agent, in one example herein a FAAH-inhibiting agent, according to the invention include cancer pain, postoperative pain, GI tract pain, spinal cord injury pain, visceral hyperalgesia, thalamic pain, headache (including stress headache and migraine), low back pain, neck pain, musculoskeletal pain, peripheral neuropathic pain, central neuropathic pain, neurogenerative disorder related pain, and menstrual pain. HIV wasting syndrome includes associated symptoms such as appetite loss and nausea. Parkinson's disease includes, for example, levodopa-induced dyskinesia. Treatment of multiple sclerosis may include treatment of symptoms such as spasticity, neurogenic pain, central pain, or bladder dysfunction. Symptoms of drug withdrawal may be caused by, for example, addiction to opiates or nicotine. Nausea or emesis may be due to chemotherapy, postoperative, or opioid related causes. Treatment of sexual dysfunction may include improving libido or delaying ejaculation. Treatment of cancer may include treatment of glioma. Sleep disorders include, for example, sleep apnea, insomnia, and disorders calling for treatment with an agent having a sedative or narcotic-type effect. Eating disorders include, for example, anorexia or appetite loss associated with a disease such as cancer or HIV infection/AIDS.

In treatment methods according to the invention, an effective amount of at least one active agent according to the invention is administered to a subject suffering from or diagnosed as having such a disease, disorder, or condition. A “therapeutically effective amount” or “effective amount” means an amount or dose of a FAAH-modulating agent sufficient to generally bring about a therapeutic benefit in patients in need of treatment for a disease, disorder, or condition mediated by FAAH activity. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.0001 to about 200 mg of active agent per kg of subject's body weight per day, preferably about 0.001 to 100 mg/kg/day, or about 0.01 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 5 g/day. Once improvement of the patient's disease, disorder, or condition has occurred, the dose may be adjusted for maintenance treatment. For example, the dosage or the frequency of administration, or both, may be reduced as a function of the symptoms, to a level at which the desired therapeutic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be coadministered separately with an active agent of Formula (I) or included with such an agent in a pharmaceutical composition according to the invention. In an exemplary embodiment, additional active ingredients are those that are known or discovered to be effective in the treatment of conditions, disorders, or diseases mediated by FAAH activity, such as another FAAH modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention. In one illustrative embodiment, a composition according to the invention may contain one or more additional active ingredients selected from opioids, NSAIDs (e.g., ibuprofen, cyclooxygenase-2 (COX-2) inhibitors, and naproxen), gabapentin, pregabalin, tramadol, acetaminophen, and aspirin.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 5 mg to 5 g daily, or from about 50 mg to 5 g daily, in single or divided doses. For example, a total daily dosage of about 5 mg to 5 g daily may be accomplished by dosing once, twice, three, or four times per day.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary active agents useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I).

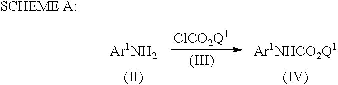

Referring to Scheme A, a carbamate of formula (IV) may be obtained by reacting a compound of formula (II) with a compound of formula (III), in which Q¹ represents an aryl group, under chloroformate condensation conditions. Preferably, Q¹ is substituted or unsubstituted phenyl, and the reaction occurs with or without a base, in a solvent such as acetonitrile, at a temperature from about 0° C. to about 80° C. More preferably, Q¹ is phenyl, and the reaction occurs in acetonitrile at about 70° C., or in the presence of a base such as pyridine, triethylamine, or diisopropylethylamine, in dichloromethane at 0° C. followed by warming to room temperature.

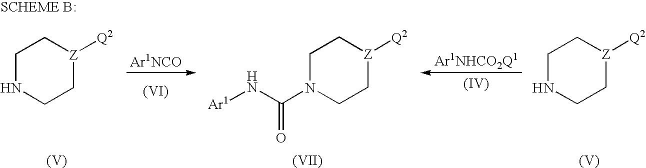

Referring to Scheme B, a compound of formula (VII) is prepared from a compound of formula (V). The group Q² is CH₂Ar² or when Z is N, Q² may also be a suitable nitrogen protecting group Q³. A compound of formula (VII) is obtained by reacting a compound of formula (V) with a compound of formula (VI) under isocyanate addition conditions. In a preferred embodiment, the reaction is performed in a solvent at a temperature from 0° C. to 100° C. Preferred conditions employ dichloromethane (DCM) at room temperature. Alternatively, a compound of formula (VII) is obtained by reacting a compound of formula (V) with a compound of formula (IV) under aryl carbamate condensation conditions. The reaction may preferably take place in a solvent at a temperature from about room temperature to about 120° C. Preferably, Q¹ is phenyl, and the reaction is performed in dimethylsulfoxide (DMSO) in a microwave reactor at about 100° C. or by conventional heating from about room temperature to about 50° C. Where Q² is CH₂Ar², compounds of formula (VII) fall within the scope of Formula (I).

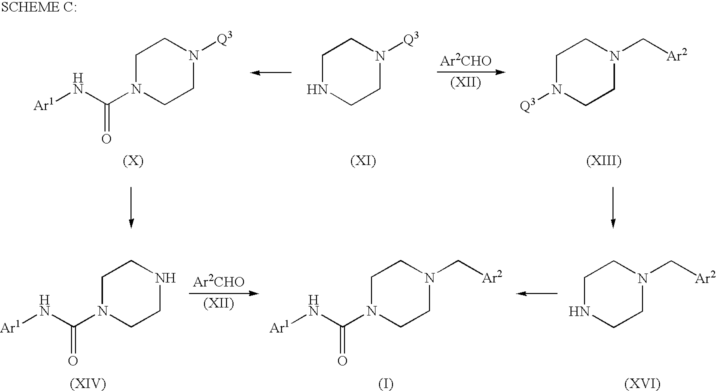

Referring to Scheme C, compounds of formula (I) are prepared from compounds of formula (XI). A suitable protecting group Q³ compatible with the transformations in Scheme C is selected. Preferably, Q³ is tert-butyl-carbamoyl (Boc). A compound of formula (X) is obtained by reacting a compound of formula (XI): (a) with a compound of formula (VI); (b) with a compound of formula (IV); or (c) with a compound Ar¹NH₂ in the presence of di-(N-succinimidyl)carbonate. An amine of formula (XIV) is obtained by deprotecting a compound of formula (X) with a reagent under suitable Q³ deprotection conditions. Boc deprotection may be preferably effected with HCl or trifluoroacetic acid (TFA) in a solvent such as diethyl ether (Et₂O), DCM, or 1,4-dioxane. A compound of Formula (I) is obtained by reacting a compound of formula (XIV) with an aldehyde (XII) under reductive amination conditions in the presence of a reductant such as sodium triacetoxyborohydride, resin-supported triacetoxyborohydride (e.g., MP—B(OAc)₃H), sodium cyanoborohydride, or phenylsilane in a solvent such as tetrahydrofuran (THF), 1,2-dichloroethane (DCE), DCM, methanol (MeOH), ethanol (EtOH), or Et₂O at a temperature from about 0° C. to 80° C. The use of a promoter or catalyst with acidic character such as an organometallic complex or carboxylic acid may increase the rate of the reaction and/or reduce the formation of by-products. Preferably, sodium triacetoxyborohydride in DCE is employed at room temperature. Reductive amination may also be performed using solid-supported triacetoxyborohydride in the presence of Et₃N in tetrahydrofuran (THF).

Alternatively, a compound of formula (XIII) is obtained by reacting an aldehyde (XI) with a protected piperazine (XII) under reductive amination conditions as described. Deprotection of Q³ from a compound of formula (XIII) under general deprotection conditions provides piperazines (XVI). A compound of Formula (I) is obtained by reacting a compound of formula (XVI) with either a compound of formula (IV) or with a compound of formula (VI) as described in the preceding schemes.

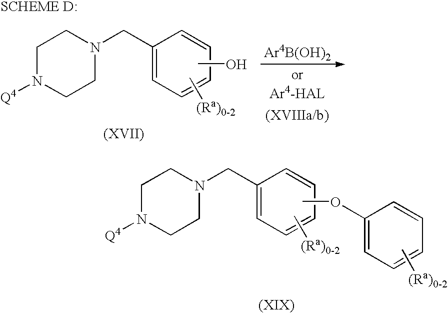

Referring to Scheme D, a compound of formula (XVII), where Q⁴ is —CONR¹Ar¹ or a nitrogen protecting group Q³, is prepared as described in the preceding schemes. A compound of formula (XVII) is converted to a compound of formula (XIX) by reaction with a suitable boronic acid (XVIIIa) in the presence of a drying agent such as powdered 4 Å molecular sieves, a promoter such as copper(II) acetate, optionally in the presence of air or a pure oxygen atmosphere, and optionally in the presence of a base such as pyridine or triethylamine, in a solvent such as DCM or DCE. Where Q⁴ is —CONR¹Ar¹, compounds of formula (XIX) are within the scope of Formula (I). Alternatively, a compound of formula (XIX), where Q⁴ is a nitrogen protecting group Q³, is prepared from (XVII) by treatment with a suitable aryl halide (XVIIIb, where HAL is chloro, bromo, or iodo) and a base such as Cs₂CO₃ in a solvent such as DMSO at temperatures ranging from about room temperature to about 120° C.

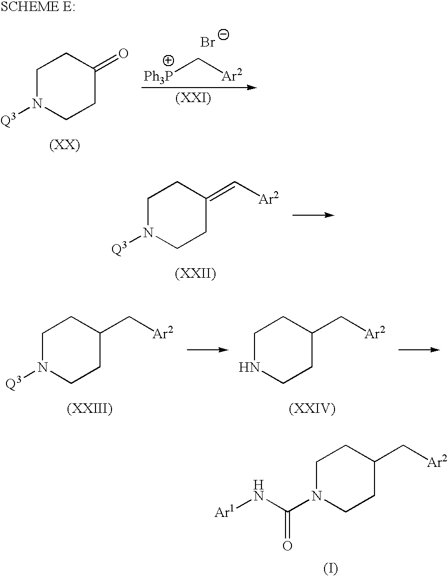

Compounds of Formula (I) are also prepared according to Scheme E. Deprotonation of a Wittig reagent (XXI; obtained from a commercial source or prepared from a suitable bromide, alcohol, aldehyde, or other precursor following general technqiues known in the art) with a base such as NaH, in a solvent such as DMSO, and subsequent treatment with a piperidone (XX), where Q³ is a nitrogen protecting group (such as Boc or benzyl) gives a compound of formula (XXII). Reduction of the double bond with hydrogen (about 10 to 100 psi) in the presence of a catalyst such as palladium on carbon or platinum(II) oxide, in solvent such as MeOH or EtOH gives a compound of formula (XXIII). Deprotection of Q³ is accomplished using conventional conditions to give a piperidine (XXIV). A compound of Formula (I) is prepared by reacting a compound of formula (XXIV) with either a compound of formula (IV) or a compound of formula (VI) as described in the preceding schemes.

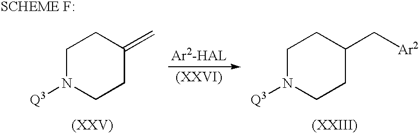

Intermediate compounds of formula (XXIII) are also prepared according to Scheme F. Hydrometallation of an alkenyl compound (XXV) gives an activated species, which is subsequently reacted with a suitable reagent Ar²-HAL (where HAL is chloride, bromide, or iodide), to provide a compound (XXIII). Preferably, hydrometallation is accomplished by hydroboration using a suitable dialkylborane reagent such as 9-borabicyclo[3.3.1]nonane (9-BBN) or diisopinocampheylborane, in a solvent such as THF. The resulting boron adduct is preferably reacted with Ar²-HAL in the presence of a suitable palladium(II) catalyst, a base such as K₂CO₃ or Cs₂CO₃, in a solvent such as N,N-dimethylformamide (DMF) or an aqueous mixture thereof. Compounds of formula (XXIII) are converted to compounds of Formula (I) using methods described in the preceding schemes.

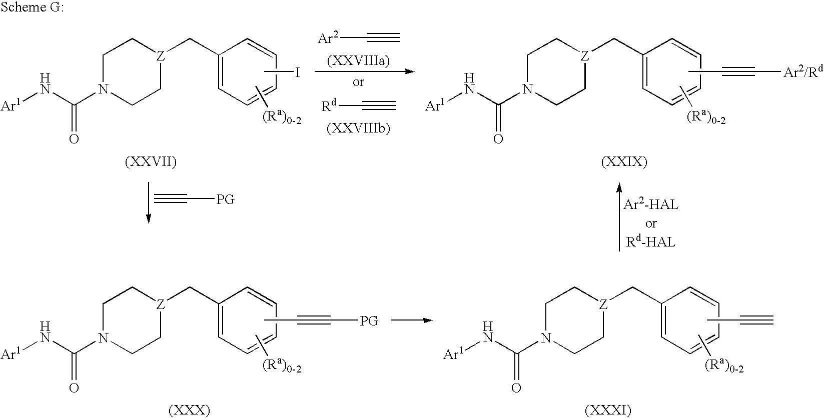

Further embodiments of Formula (I), such as compounds (XXIX) or (XXXI) are prepared as described in Scheme G. Palladium-catalyzed coupling of compounds (XXVII, prepared as described in the preceding Schemes) with alkynes (XXVIIIa/b) provides compounds (XXIX). Preferably, reactions are run in the presence of a palladium(II) catalyst such as Pd(PPh₃)₂Cl₂, a copper(I) catalyst such as Cul, a base such as triethylamine, with or without an additional phosphine ligand such as triphenylphosphine, in a solvent such as THF, at a temperature from about room temperature to about 50° C. Alternatively, iodides (XXVII) are coupled with a protected alkyne reagent, where PG is a suitable protecing group such as trimethylsilyl, to give compounds (XXX). Removal of the protecting group gives compounds (XXXI). A second palladium-catalyzed coupling reaction with suitable halides Ar²-HAL or R^{d}-HAL (where HAL is chloride, bromide, or iodide) gives compounds (XXIX).

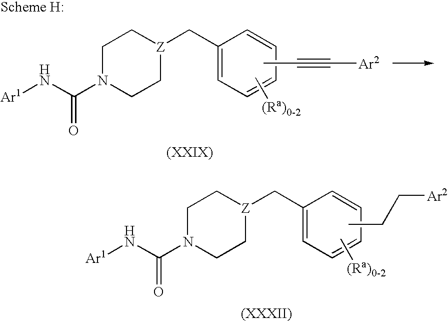

Alkynes (XXIX) are optionally reduced to compounds (XXXII) using standard hydrogenation protocols. Preferably, reactions are accomplished using hydrogen gas and a catalyst such as palladium on carbon, in a solvent such as EtOH.

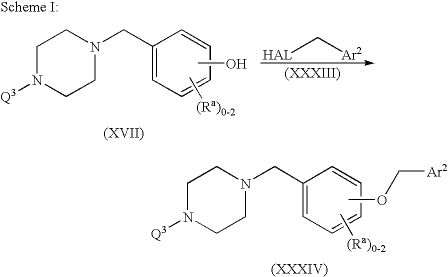

Further embodiments of Formula (I) are prepared as shown in Scheme I. Alkylation of a phenol (XVII) with a suitable benzyl halide (XXXIII) and a base such as K₂CO₃ in a solvent such as acetonitrile at a temperature from about room temperature to about 50° C. provides compounds (XXXIV).

Compounds of Formula (I) may be converted to their corresponding salts by applying general techniques described in the art. For example, a compound of Formula (I) may be treated with trifluoroacetic acid, HCl, or citric acid in a solvent such as Et₂O, 1,4-dioxane, DCM, THF, or MeOH to provide the corresponding salt forms.

Compounds prepared according to the schemes described above may be obtained as single enantiomers, diastereomers, or regioisomers, by enantio-, diastero-, or regio-specific synthesis, or by resolution. Compounds prepared according to the schemes above may alternatively be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following specific examples are provided to further illustrate the invention and various preferred embodiments.

### EXAMPLES

### Chemistry:

In preparing the examples listed below, the following general experimental and analytical methods were used.

Reaction mixtures were stirred under a nitrogen atmosphere at room temperature (rt) unless otherwise noted. Where solutions or mixtures are concentrated, they are typically concentrated under reduced pressure using a rotary evaporator. Where solutions are dried, they are typically dried over a drying agent such as MgSO₄ or Na₂SO₄.

Normal phase flash column chromatography (FCC) was performed on silica gel columns using ethyl acetate (EtOAc)/hexanes as eluent, unless otherwise indicated.

Reversed-Phase high performance liquid chromatography (HPLC) was performed using: 1) a Gilson® instrument with a YMC-Pack ODS-A, 5 μm, 75×30 mm column, a flow rate of 25 mL/min, detection at 220 and 254 nm, with a 15% to 99% acetonitrile/water/0.05% TFA gradient; or 2) Shimadzu instrument with a Phenomenex Gemini column 5 μm C18 (150×21.2 mm) or Waters Xterra RP18 OBD column 5 μm (100×30 mm), a gradient of 95:5 to 0:100 water (0.05% TFA)/CH₃CN (0.05% TFA), a flow rate of 30 mL/min, and detection at 254 nM.

Mass spectra were obtained on an Agilent series 1100 MSD using electrospray ionization (ESI) in positive mode unless otherwise indicated.

NMR spectra were obtained on either a Bruker model DPX400 (400 MHz), DPX500 (500 MHz), DRX600 (600 MHz) spectrometer. The format of the ¹H NMR data below is: chemical shift in ppm down field of the tetramethylsilane reference (multiplicity, coupling constant J in Hz, integration).

Chemical names were generated using ChemDraw Ultra 6.0.2 (CambridgeSoft Corp., Cambridge, Mass.) or ACD/Name Version 9 (Advanced Chemistry Development, Toronto, Ontario, Canada).

### Example 1

### 4-(2,2-Difluoro-benzo[1,3]dioxol-5-ylmethyl)-piperazine-1-carboxylic acid benzo[d]isoxazol-3-ylamide trifluoroacetic acid salt

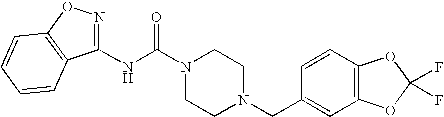

Step A: Benzo[d]isoxazol-3-yl-carbamic acid phenyl ester. A mixture of benzo[d]isoxazol-3-ylamine (3.0 g) and ClCO₂Ph (0.94 mL) in dry CH₃CN (30 mL) was stirred for 23 h at 70° C. The reaction mixture was poured into de-ionized water, stirred for 30 min and filtered. The isolated solid was rinsed thoroughly with water and then dried under high vacuum to give 1.90 g (100%) of the title compound. MS: 255.1.

Step B: 1-(2,2-Difluoro-benzo[1,3]dioxol-5-ylmethyl)-piperazine. A 0° C. solution of piperazine-1-carboxylic acid tert-butyl ester (20.0 g) and 2,2-difluoro-benzo[1,3]dioxole-5-carbaldehyde (14.8 mL) in DCE (208 mL) was treated with NaB(OAc)₃H (31.8 g). The mixture was allowed to warm to rt and was stirred for 16 h. The resulting mixture was cooled in an ice bath and treated with 10% aq. KOH (200 mL). After 1 h, the resulting mixture was extracted with DCM (3×200 mL). The combined organic extracts were dried and concentrated, giving a white solid (37.6 g). This solid was dissolved in MeOH (850 mL) and treated with HCl (2 M in Et₂O; 159 mL). After 16 h, the resulting mixture was treated with Et₂O (850 mL). A white precipitate was filtered off and washed with Et₂O (2×140 mL), giving a white solid (27.6 g). This solid (27.5 g) was suspended in DCM (200 mL) and treated with 10% aq. KOH (200 mL). The organic phase was extracted with DCM (2×150 mL). The combined organic extracts were dried and concentrated, giving the title compound as a white solid (20.8 g). MS: 257.1.

Step C: 4-(2,2-Difluoro-benzo[1,3]dioxol-5-ylmethyl)-piperazine-1-carboxylic acid benzo[d]isoxazol-3-ylamide trifluoroacetic acid salt. To a Smith Process vial were added a spin vane, benzo[d]isoxazol-3-yl-carbamic acid phenyl ester (51.2 mg), 1-(2,2-difluoro-benzo[1,3]dioxol-5-ylmethyl)-piperazine (76.5 mg) and DMSO (0.5 mL). The vial was purged with N₂, capped and heated via microwave irradiation for 15 min at 100° C. The reaction mixture was then directly purified by reverse-phase HPLC to give 62.4 mg (58%) of the desired product as the TFA salt. MS: 417.2. ¹H NMR (d₄-MeOH): 7.88 (d, J=7.8, 1H), 7.60-7.57 (m, 1H), 7.52 (d, J=8.4, 1H), 7.43 (d, J=1.8, 1H), 7.35-7.34 (dd, J=1.5, 8.1, 1H), 7.32-7.30 (m, 2H), 4.53-3.54 (br hump, 4H), 4.43 (s, 2H), 3.40 (br s, 4H).

The compounds in Examples 2-8 were prepared using methods analogous to those described in Example 1.

### Example 2

### 4-(2,2-Difluoro-benzo[1,3]dioxol-5-ylmethyl)-piperazine-1-carboxylic acid (3-phenyl-[1,2,4]thiadiazol-5-yl)-amide trifluoroacetic acid salt

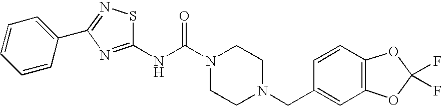

MS: 460.5. ¹H NMR (CDCl₃): 10.66 (br s, 1H), 8.10-8.08 (m, 2H), 7.46-7.43 (m, 3H), 7.00 (s, 1H), 6.96 (d, J=8.4, 1H), 6.91-6.89 (dd, J=1.2, 7.8, 1H), 3.33 (br s, 6H), 2.15 (br s, 4H).

### Example 3

### 4-(2,2-Difluoro-benzo[1,3]dioxol-5-ylmethyl)-piperazine-1-carboxylic acid (1H-tetrazol-5-yl)-amide trifluoroacetic acid salt

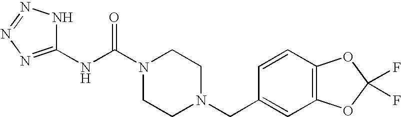

MS: 368.5. ¹H NMR (d₆-DMSO): 15.51 (s, 1H), 10.98 (s, 1H), 7.54 (s, 2H), 7.33-7.32 (m, 1H), 4.29 (br s, 4H), 3.58-2.86 (m, 6H).

### Example 4

### 4-(2,2-Difluoro-benzo[1,3]dioxol-5-ylmethyl)-piperazine-1-carboxylic acid benzo[1,2,5]thiadiazol-4-ylamide trifluoroacetic acid salt

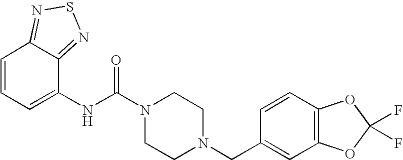

MS: 434.5. ¹H NMR (d₄-MeOH): 7.94-7.93 (dd, J=1.2, 7.2, 1H), 7.63-7.57 (m, 2H), 7.26 (s, 1H), 7.14 (d, J=0.6, 2H), 3.64 (t, J=4.8, 4H), 2.54 (t, J=4.8, 4H).

### Example 5

### 4-(2,2-Difluoro-benzo[1,3]dioxol-5-ylmethyl)-piperazine-1-carboxylic acid benzo[1,2,5]oxadiazol-4-ylamide trifluoroacetic acid salt

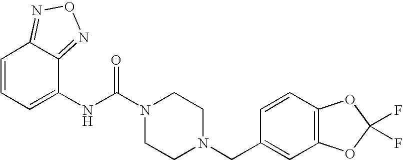

MS: 418.2. ¹H NMR (d₄-MeOH): 7.62 (d, J=7.2, 1H), 7.57 (d, J=9.0, 1H), 7.48-7.45 (m, 2H), 7.38-7.34 (m, 2H), 4.44 (s, 2H), 4.28-3.63 (br hump, 4H), 3.39 (br s, 4H).

### Example 6

### 4-(2,2-Difluoro-benzo[1,3]dioxol-5-ylmethyl)-piperazine-1-carboxylic acid (3H-benzotriazol-5-yl)-amide trifluoroacetic acid salt

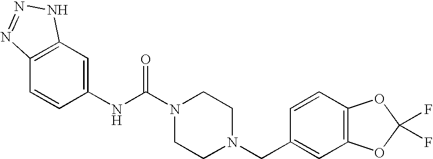

MS: 417.2. ¹H NMR (d₄-MeOH): 8.00 (s, 1H), 7.79 (d, J=9.0, 1H), 7.44 (s, 1H), 7.42-7.40 (dd, J=1.8, 9.0, 1H), 7.37-7.33 (m, 2H), 4.44 (s, 2H), 4.50-3.20 (br hump, 4H), 3.37 (br s, 4H).

### Example 7

### 4-(2,2-Difluoro-benzo[1,3]dioxol-5-ylmethyl)-piperazine-1-carboxylic acid thiophen-2-ylamide

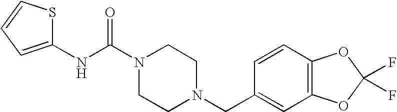

MS: 382.1. ¹H NMR (CDCl₃): 7.11 (s, 1H), 7.05 (s, 1H), 7.02-6.97 (m, 2H), 6.83-6.77 (m, 2H), 6.54-6.51 (m, 1H), 3.52-3.48 (m, 6H), 2.49-2.43 (m, 4H).

### Example 8

### 4-(2,2-Difluoro-benzo[1,3]dioxol-5-ylmethyl)-piperazine-1-carboxylic acid thiophen-3-ylamide

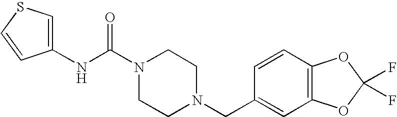

MS: 382.1. ¹H NMR (CDCl₃): 7.27-7.25 (m, 1H), 7.21-7.17 (m, 1H), 7.11 (s, 1H), 7.01-6.94 (m, 3H), 6.69 (s, 1H), 3.51-3.45 (m, 6H), 2.48-2.42 (m, 4H).

### Example 9

### 4-Naphthalen-2-ylmethyl-piperidine-1-carboxylic acid (6-chloro-pyridazin-3-yl)-amide

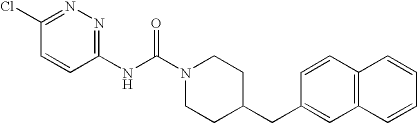

Step A: Naphthalen-2-ylmethyl-triphenyl-phosphonium bromide. A flask containing a mixture of 2-bromomethyl-naphthalene (25.0 g) and triphenylphosphine (31.3 g) in xylenes (230 mL) was fitted with a reflux condenser, purged with N₂, and heated to 135° C. for 24 h. The resulting white solid was isolated by filtration, washed with toluene, and dried under high vacuum.

Step B: 4-Naphthalen-2-ylmethylene-piperidine-1-carboxylic acid tert-butyl ester. A 0° C. suspension of NaH (95%, 3.30 g) in dry DMSO (300 mL) was stirred for 10 min and then treated with a hot solution of naphthalen-2-ylmethyl-triphenyl-phosphonium bromide (52.4 g) in DMSO (100 mL) via cannula over 20 min (heating the DMSO solution was necessary to dissolve the phosphonium salt). The resultant bright red mixture was allowed to stir at 0° C. for 10 min before adding a solution of N-Boc-piperidinone (26.3 g) in DMSO (100 mL) via cannula over 20 min. After stirring for 1 h at 0° C., 3 h at rt and 50° C. for 18 h, the mixture was diluted with 1-L water and extracted with Et₂O (500 mL×4). The organic extracts were washed with water (×2), dried, and concentrated to give a yellow heterogeneous mixture. The crude material was suspended in hot hexanes (700 mL) and the solid removed by filtration. Concentration of the filtrate gave an oily residue that was purified by FCC to give 28.1 g (80%) of the title compound as a colorless oil.

Step C: 4-Naphthalen-2-ylmethyl-piperidine-1-carboxylic acid tert-butyl ester. A flask containing a suspension of 4-naphthalen-2-ylmethylene-piperidine-1-carboxylic acid tert-butyl ester (22.7 g) and 10% Pd/C (6.3 g) in EtOH (350 mL) was evacuated and then affixed with a H₂ balloon. After 18 h, the H₂ was evacuated from the flask and replaced with N₂. The reaction mixture was filtered twice through diatomaceous earth and then through a Zapcap. The filtrate was concentrated to give 21.9 g (96%) of the title compound as a pale-yellow oil. MS: 348.5 (M+Na)⁺.

Step D: 4-Naphthalen-2-ylmethyl-piperidine. A mixture of 4-naphthalen-2-ylmethyl-piperidine-1-carboxylic acid tert-butyl ester (21.4 g) and TFA (75 mL) was stirred at rt for 18 h. The mixture was concentrated, diluted with DCM, and washed with 1 N NaOH. The organic layer was dried and concentrated to give 14.8 g (100%) of the title compound as a pale-yellow oil that crystallized upon standing. MS: 226.2.

Step E: 4-Naphthalen-2-ylmethyl-piperidine-1-carboxylic acid (6-chloro-pyridazin-3-yl)-amide. The title compound was prepared using methods analogous to those described in Example 1, Step C. MS: 481.5. ¹H NMR (CDCl₃): 8.46 (br s, 1H), 8.31 (br s, 1H), 7.81 (d, J=7.5, 1H), 7.78 (d, J=8.5, 2H), 7.58 (s, 1H), 7.48-7.39 (m, 3H), 7.30-7.28 (dd, J=1.5, 8.5, 1H), 4.17 (d, J=13.5, 2H), 2.87 (t, J=12.5, 2H), 2.73 (d, J=7.0, 2H), 1.94-1.83 (m, 1H), 1.76 (d, J=13, 2H), 1.36-1.25 (m, 2H).

The compounds in Examples 10-27 were prepared using methods analogous to those described in Example 9.

### Example 10

### 4-Naphthalen-2-ylmethyl-piperidine-1-carboxylic acid pyrazin-2-ylamide trifluoroacetic acid salt

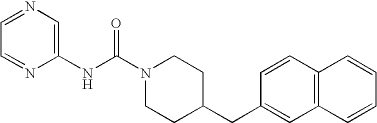

MS: 347.5. ¹H NMR (CDCl₃): 9.42 (d, J=1.5, 1H), 8.27 (d, J=2.5, 1H), 8.15-8.14 (dd, J=1.5, 2.5, 1H), 7.84-7.79 (m, 3H), 7.60 (s, 1H), 7.48-7.43 (m, 2H), 7.32-7.30 (m, 1H), 4.13 (d, J=13, 2H), 2.94-2.88 (dt, J=3.0, 12.5, 2H), 2.76 (d, J=7.5, 2H), 1.92-1.87 (m, 1H), 1.81 (d, J=13.0, 2H), 1.39-1.30 (m, 2H).

### Example 11

### 4-Naphthalen-2-ylmethyl-piperidine-1-carboxylic acid isoxazol-3-ylamide

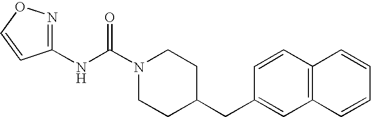

MS: 336.5. ¹H NMR (d₆-acetone): 8.71 (s, 1H), 8.16 (s, 1H), 7.81 (d, J=8.4, 1 H), 7.78 (d, J=8.4, 2H), 7.58 (s, 1H), 7.48-7.42 (m, 2H), 7.30-7.28 (dd, J=1.8, 9, 1H), 7.00 (s, 1H), 4.17 (d, J=13.2, 2H), 2.86 (t, J=12.6, 2H), 2.72 (d, J=7.2, 2H), 1.90-1.83 (m, 1H), 1.75 (d, J=12.6, 2H), 1.33-1.25 (m, 2H).

### Example 12

### 4-Naphthalen-2-ylmethyl-piperidine-1-carboxylic acid (3-phenyl-[1,2,4]thiadiazol-5-yl)-amide

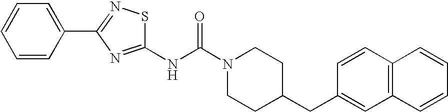

MS: 429.5. ¹H NMR (CDCl₃): 8.13-8.12 (m, 2H), 7.82 (d, J=7.8, 1H), 7.79 (d, J=8.4, 2H), 7.56 (s, 1H), 7.50-7.43 (m, 5H), 7.28-7.26 (m, 1H), 4.16 (br s, 1H), 2.86 (t, J=12.6, 2H), 2.70 (d, J=7.2, 2H), 1.90-1.83 (m, 1H), 1.76 (d, J=13.2, 2H), 1.28-1.21 (m, 2H).

### Example 13

### 4-Naphthalen-2-ylmethyl-piperidine-1-carboxylic acid (1H-tetrazol-5-yl)-amide

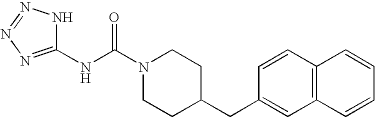

MS: 337.5. ¹H NMR (d₆-DMSO): 15.36 (s, 1H), 10.67 (s, 1H), 7.88-7.84 (m, 3H), 7.68 (s, 1H), 7.50-7.44 (m, 2H), 7.38-7.37 (dd, J=1.8, 8.4, 1H), 4.15 (d, J=13.2, 2H), 2.83-2.79 (m, 2H), 2.70 (d, J=7.2, 2H), 1.91-1.85 (m, 1H), 1.64-1.61 (m, 2H), 1.20-1.13 (m, 3H).

### Example 14

### 4-Naphthalen-2-ylmethyl-piperidine-1-carboxylic acid (2H-pyrazol-3-yl)-amide

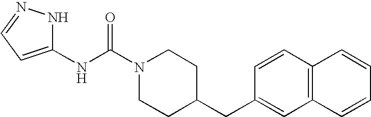

MS: 335.5. ¹H NMR (CDCl₃): 7.79 (d, J=7.8, 1H), 7.76 (d, J=7.8, 2H), 7.63 (br s, 1H), 7.56 (s, 1H), 7.46-7.40 (m, 2H), 7.37 (s, 1H), 7.28-7.26 (m, 1H), 6.37 (br s 1H), 4.05 (d, J=12.6, 2H), 2.78-2.74 (m, 2H), 2.68 (d, J=7.2, 2H), 1.83-1.77 (m, 1H), 1.68 (d, J=12.6, 2H), 1.29-1.21 (m, 2H).

### Example 15

### 4-Naphthalen-2-ylmethyl-piperidine-1-carboxylic acid benzo[1,2,5]oxadiazol-4-ylamide

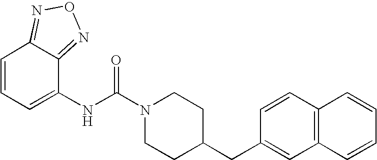

MS: 387.3. ¹H NMR (CDCl₃): 7.97-7.96 (m, 1H), 7.82 (d, J=15.6, 1H), 7.80-7.81 (m, 2H), 7.60 (s, 1H), 7.49-7.43 (m, 2H), 7.40-7.39 (m, 2H), 7.36 (s, 1H), 7.30 (d, J=8.4, 1H), 4.13 (d, J=13.2, 2H), 2.98-2.94 (m, 2H), 2.76 (d, J=7.2, 2H), 1.96-1.88 (m, 1H), 1.85-1.82 (m, 2H), 1.40-1.33 (m, 2H).

### Example 16

### 4-Naphthalen-2-ylmethyl-piperidine-1-carboxylic acid (1H-benzotriazol-5-yl)-amide

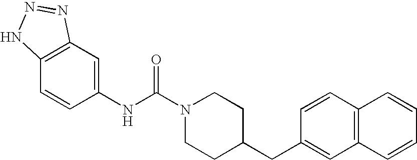

MS: 386.3. ¹H NMR (CDCl₃): 8.19 (br s, 1H), 7.80-7.76 (m, 3H), 7.64 (br s, 1H), 7.56 (s, 1H), 7.46-7.40 (m, 2H), 7.36 (br s, 1H), 6.91 (br s, 1H), 4.14 (d, J=11.4, 2H), 2.91-2.87 (m, 2H), 2.69 (d, J=6.6, 2H), 1.90 (br s, 1H), 1.77 (d, J=12.6, 2H), 1.34-1.28 (m, 2H).

### Example 17

### 4-Naphthalen-2-ylmethyl-piperidine-1-carboxylic acid [1,5]naphthyridin-2-ylamide trifluoroacetic acid salt

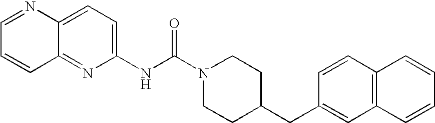

MS: 397.3. ¹H NMR (CDCl₃): 9.00 (d, J=3.6, 1H), 8.65 (d, J=9.6, 1H), 8.49 (d, J=9.6, 1H), 8.41 (d, J=8.4, 1H), 7.82-7.78 (m, 4H), 7.59 (s, 1H), 7.48-7.42 (m, 2H), 7.31-7.29 (dd, J=1.2, 8.4, 1H), 4.32-4.30 (m, 2H), 2.96 (br s, 2H), 2.76 (d, J=7.2, 2H), 1.96-1.88 (m, 1H), 1.84 (d, J=13.2, 2H), 1.41-1.34 (m, 2H).

### Example 18

### 4-Naphthalen-2-ylmethyl-piperidine-1-carboxylic acid quinolin-2-ylamide trifluoroacetic acid salt

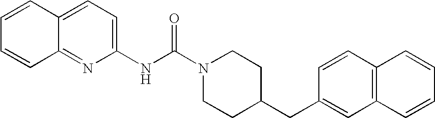

MS: 396.3. ¹H NMR (d₆-acetone): 8.72 (d, J=9.6, 1H), 8.23 (d, J=9.0, 1H), 8.12-8.09 (m, 2H), 7.99-7.96 (m, 1H), 7.87-7.83 (m, 3H), 7.73-7.70 (m, 2H), 7.49-7.43 (m, 2H), 7.41-7.40 (dd, J=1.2, 8.4, 1H), 4.35 (d, J=13.8, 2H), 2.99 (br s, 2H), 2.78 (d, J=7.2, 2H), 2.09-1.98 (m, 1H), 1.80-1.77 (m, 2H), 1.41-1.34 (m, 2H).

### Example 19

### 4-Naphthalen-2-ylmethyl-piperidine-1-carboxylic acid benzothiazol-6-ylamide

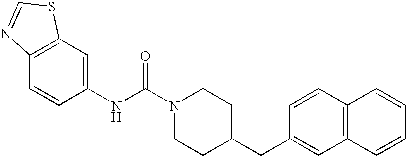

MS: 402.2. ¹H NMR (CDCl₃): 9.01 (s, 1H), 8.31 (s, 1H), 8.03 (d, J=8.4, 1H), 7.83-7.78 (m, 3H), 7.59 (s, 1H), 7.49-7.43 (m, 2H), 7.30 (d, J=8.4, 1H), 7.25-7.23 (m, 1H), 6.70 (s, 1H), 4.08 (d, J=13.8, 2H), 2.91-2.86 (m, 2H), 2.75 (d, J=7.2, 2H), 1.93-1.85 (m, 1H), 1.78 (d, J=12.6, 2H), 1.37-1.30 (m, 2H).

### Example 20

### 4-Naphthalen-2-ylmethyl-piperidine-1-carboxylic acid quinolin-5-ylamide trifluoroacetic acid salt

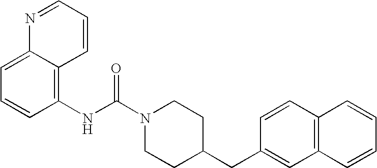

MS: 396.3. ¹H NMR (CDCl₃): 8.72 (d, J=7.8, 2H), 7.95 (s, 1H), 7.85-7.80 (m, 4H), 7.66-7.62 (m, 3H), 7.52-7.44 (m, 3H), 7.32 (d, J=8.4, 1H), 4.24 (d, J=13.8, 2H), 2.93 (t, J=12.6, 2H), 2.78 (d, J=6.6, 2H), 1.96-1.87 (m, 1H), 1.82 (d, J=12.6, 2H), 1.41-1.34 (m, 2H).

### Example 21

### 4-Naphthalen-2-ylmethyl-piperidine-1-carboxylic acid benzo[d]isoxazol-3-ylamide

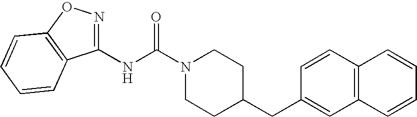

MS: 386.3. ¹H NMR (CDCl₃/d₄-MeOH mix): 7.84 (d, J=7.8, 1H), 7.79-7.75 (m, 3H), 7.58 (s, 1H), 7.52-7.49 (m, 1H), 7.44-7.38 (m, 3H), 7.31-7.29 (dd, J=1.5, 15.9, 1H), 7.24 (br t, J=7.5, 1H), 4.18 (d, J=13.2, 2H), 2.91-2.87 (m, 2H), 2.73 (d, J=7.2, 2H), 1.95-1.85 (m, 1H), 1.75 (d, J=12.6, 2H), 1.36-1.29 (m, 2H).

### Example 22

### 4-(4-Fluoro-benzyl)-piperidine-1-carboxylic acid benzo[d]isoxazol-3-ylamide

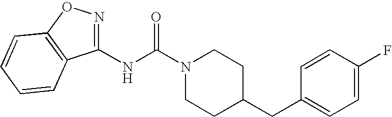

MS: 354.2. ¹H NMR (CDCl₃): 8.79 (s, 1H), 8.06 (d, J=8.5, 1H), 7.54-7.51 (m, 1H), 7.43-7.42 (m, 1H), 7.29-7.26 (m, 1H), 7.11-7.08 (m, 2H), 7.00-6.96 (m, 2H), 4.27 (d, J=13.0, 2H), 2.93 (t, J=12.0, 2H), 2.55 (d, J=7.0, 2H), 1.74 (d, J=9.5, 3H), 1.33-1.24 (m, 2H).

### Example 23

### 4-(4-Fluoro-benzyl)-piperidine-1-carboxylic acid (6-chloro-pyridazin-3-yl)-amide

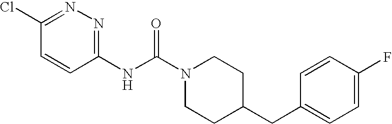

MS: 349.2. ¹H NMR (CDCl₃): 8.45 (s, 1H), 8.29 (d, J=9.5, 1H), 7.42 (d, J=9.5, 1H), 7.11-7.08 (m, 2H), 6.99-6.96 (m, 2H), 4.18 (d, J=13.5, 2H), 2.90-2.89 (m, 2H), 2.55 (d, J=6.5, 2H), 1.77-1.71 (m, 3H), 1.29-1.21 (m, 2H).

### Example 24

### 4-(4-Fluoro-benzyl)-piperidine-1-carboxylic acid isoxazol-3-ylamide

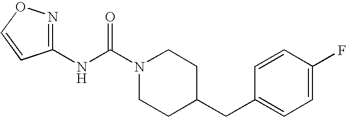

MS: 304.2. ¹H NMR (CDCl₃): 9.04 (s, 1H), 8.17 (d, J=1.5, 1H), 7.10-7.08 (m, 2H), 7.01 (d, J=2.0, 1H), 6.99-6.96 (m, 2H), 4.21 (m, J=13.5, 2H), 2.88-2.83 (m, 2H), 2.33 (d, J=7.0, 2H), 1.74-1.70 (m, 3H), 1.28-1.93 (m, 2H).

### Example 25

### 4-(3-Trifluoromethyl-benzyl)-piperidine-1-carboxylic acid (6-chloro-pyridazin-3-yl)-amide

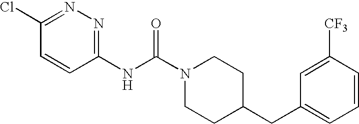

MS: 399.1. ¹H NMR (CDCl₃): 8.46 (d, J=9.5, 1H), 7.57 (d, J=9.5, 1H), 7.48 (d, J=8.0, 1H), 7.43-7.40 (m, 2H), 7.33 (d, J=7.5, 1H), 4.22 (d, J=13.5, 2H), 2.90 (t, J=12.0, 2H), 2.64 (d, J=7.0, 2H), 1.86-1.74 (m, 3H), 1.33-1.25 (m, 2H).

### Example 26

### 4-(3-Trifluoromethyl-benzyl)-piperidine-1-carboxylic acid isoxazol-3-ylamide

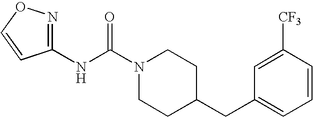

MS: 354.2. ¹H NMR (CDCl₃): 9.11 (s, 1H), 8.17 (d, J=2.0, 1H), 7.48 (d, J=7.5, 1H), 7.43-7.40 (m, 2H), 7.33 (d, J=7.5, 1H), 7.02 (d, J=2.0, 1H), 4.22 (d, J=13.5, 2H), 2.90-2.84 (m, 2H), 2.63 (d, J=7.0, 2H), 1.82-1.76 (m, 1H), 1.72 (d, J=13.5, 2H), 1.27 (m, 2H).

### Example 27

### 4-(3-Trifluoromethyl-benzyl)-piperidine-1-carboxylic acid benzo[d]isoxazol-3-ylamide

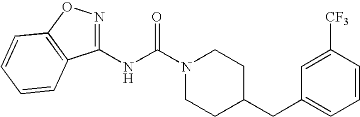

MS: 404.2. ¹H NMR (CDCl₃): 9.09 (s, 1H), 8.06 (d, J=8.0, 1H), 7.54-7.48 (m, 2H), 7.42-7.39 (m, 3H), 7.32 (d, J=7.5, 1H), 7.29-7.26 (m, 1H), 4.30 (d, J=13.0, 2H), 2.93 (t, J=12.5, 2H), 2.63 (d, J=7.0, 2H), 1.84-1.78 (m, 1H), 1.74 (d, J=14.0, 2H), 1.35-1.27 (m, 2H).

### Example 28

### 4-[3-(4-Fluoro-3-trifluoromethyl-phenoxy)-benzyl]-piperazine-1-carboxylic acid benzo[d]isoxazol-3-ylamide trifluoroacetic acid salt

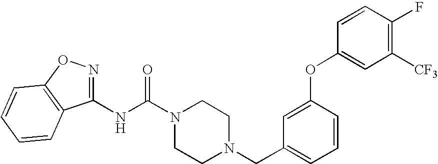

Step A: 4-(3-Hydroxy-benzyl)-piperazine-1-carboxylic acid benzo[d]isoxazol-3-ylamide. The title compound was prepared using methods analogous to those described in Example 1. MS: 353.2. ¹H NMR (d₄-MeOH): 7.84-7.81 (m, 1H), 7.59-7.54 (m, 1H), 7.53-7.50 (m, 1H), 7.32-7.27 (m, 1H), 7.16-7.11 (m, 1H), 6.82-6.80 (m, 2H), 6.71-6.68 (m, 1H), 3.64-3.61 (m, 4H), 3.52-3.50 (m, 2H), 2.55-2.51 (m, 4H).

Step B: 4-[3-(4-Fluoro-3-trifluoromethyl-phenoxy)-benzyl]-piperazine-1-carboxylic acid benzo[d]isoxazol-3-ylamide trifluoroacetic acid salt. A mixture of 4-fluoro-3-(trifluoromethyl)phenylboronic acid (124.7 mg), pyridine (122 μL), 4 Å powdered molecular sieves (181 mg), and Cu(OAc)₂ (51.5 mg) in DCM (3 mL) was stirred, open to air, for 48 h at rt. Additional DCM was added as the mixture dried up. The reaction mixture was filtered through a pad of diatomaceous earth and passed through a pad of silica gel (NH₃/MeOH/DCM). The filtrate was concentrated and the residue was purified by reverse-phase HPLC to give 45.1 mg (24%) of the desired product as the TFA salt. MS: 515.2. ¹H NMR (CDCl₃): 9.75 (s, 1H), 7.93 (d, J=8.0, 1H), 7.51-7.48 (m, 1H), 7.35-7.32 (m, 2H), 7.24 (t, J=8.0, 1H), 7.19-7.17 (m, 1H), 7.15-7.10 (m, 3H), 7.03 (t, J=2.0, 1H), 6.99-6.97 (m, 1H), 4.17 (s, 2H), 4.39-3.50 (br s, 4H), 3.45-2.82 (br s, 4H).

The compounds in Examples 29-35 were prepared using methods analogous to those described in Example 28.

### Example 29

### 4-[3-(3-Trifluoromethoxy-phenoxy)-benzyl]-piperazine-1-carboxylic acid benzo[d]isoxazol-3-ylamide trifluoroacetic acid salt

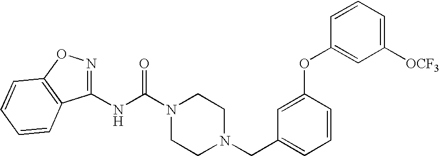

MS: 513.2. ¹H NMR (d₄-MeOH): 7.87 (d, J=8.0, 1H), 7.62-7.59 (m, 1H), 7.56-7.52 (m, 2H), 7.47 (t, J=8.0, 1H), 7.36-7.30 (m, 2H), 7.27 (s, 1H), 7.21-7.18 (m, 1H), 7.08-7.07 (m, 1H), 7.04-7.01 (m, 1H), 6.94 (s, 1H), 4.42 (s, 2H), 4.09-3.50 (br s, 4H), 3.39 (s, 4H).

### Example 30

### 4-[3-(4-Trifluoromethoxy-phenoxy)-benzyl]-piperazine-1-carboxylic acid benzo[d]isoxazol-3-ylamide trifluoroacetic acid salt

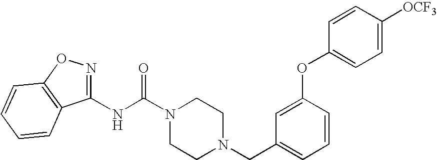

MS: 513.2. ¹H NMR (d₄-MeOH): 7.87 (d, J=8.0, 1H), 7.61-7.58 (m, 1H), 7.55-7.52 (m, 2H), 7.33-7.30 (m, 4H), 7.24-7.23 (m, 1H), 7.19-7.17 (m, 1H), 7.14-7.11 (m, 2H), 4.41 (s, 2H), 4.15-3.55 (br s, 4H), 3.38 (s, 4H).

### Example 31

### 4-[3-(3-Bromo-phenoxy)-benzyl]-piperazine-1-carboxylic acid benzo[d]isoxazol-3-ylamide trifluoroacetic acid salt

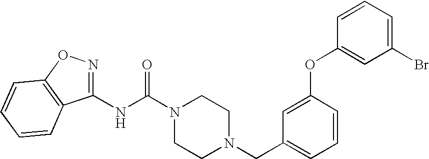

MS: 507.1. ¹H NMR (CDCl₃): 9.72 (s, 1H), 7.93 (d, J=8.0, 1H), 7.52-7.49 (m, 1H), 7.36-7.32 (m, 2H), 7.26-7.23 (m, 2H), 7.17 (t, J=8.0, 1H), 7.12 (d, J=8.0, 1H), 7.10 (t, J=2.0, 1H), 7.02-7.01 (m, 2H), 6.90-6.88 (m, 1H), 4.15 (s, 2H), 4.35-3.55 (br s, 4H), 3.55-2.89 (br s, 4H).

### Example 32

### 4-[3-(4-Bromo-phenoxy)-benzyl]-piperazine-1-carboxylic acid benzo[d]isoxazol-3-ylamide trifluoroacetic acid salt

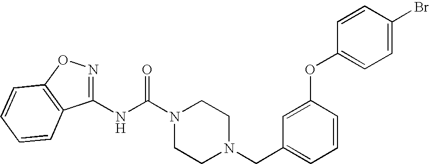

MS: 507.1. ¹H NMR (CDCl₃): 9.70 (s, 1H), 7.93 (d, J=8.0, 1H), 7.50 (t, J=8.0, 1H), 7.43-7.40 (m, 2H), 7.36-7.31 (m, 2H), 7.27-7.24 (m, 1H), 7.09 (d, J=7.5, 1H), 7.00-6.99 (m, 2H), 6.85-6.82 (m, 2H), 4.15 (s, 2H), 4.31-3.35 (br s, 4H), 3.45-2.70 (br s, 4H).

### Example 33

### 4-[3-(3,4-Difluoro-phenoxy)-benzyl]-piperazine-1-carboxylic acid benzo[d]isoxazol-3-ylamide

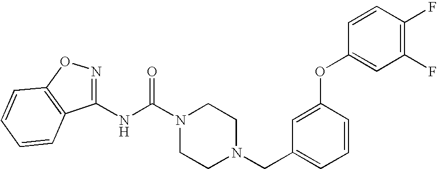

MS: 465.2. ¹H NMR (d₄-MeOH): 9.78 (s, 1H), 7.92 (d, J=6.8, 1H), 7.49 (t, J=6.4, 1H), 7.34-7.31 (m, 2H), 7.23 (t, J=6.0, 1H), 7.11-7.07 (m, 2H), 7.01-6.98 (m, 2H), 6.80-6.76 (m, 1H), 6.70-6.67 (m, 1H), 4.14 (s, 2H), 4.30-3.50 (br s, 4H), 3.35-2.85 (br s, 4H).

### Example 34

### 4-[3-(3,5-Difluoro-phenoxy)-benzyl]-piperazine-1-carboxylic acid benzo[d]isoxazol-3-ylamide trifluoroacetic acid salt

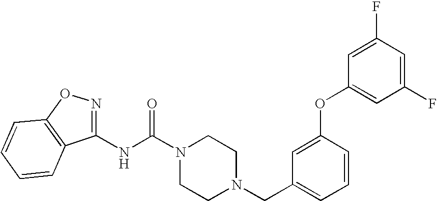

MS: 465.2. ¹H NMR (CDCl₃): 9.75 (s, 1H), 7.92 (d, J=8.0, 1H), 7.51-7.47 (m, 1H), 7.38-7.33 (m, 2H), 7.24 (t, J=8.0, 1H), 7.17 (d, J=7.5, 1H), 7.07-7.05 (m, 2H), 6.56-6.51 (m, 1H), 6.46-6.41 (m, 2H), 4.147 (s, 2H), 4.28-3.55 (br s, 4H), 3.40-2.90 (br s, 4H).

### Example 35

### 4-{3-[4-(2,2,2-Trifluoro-ethoxy)-phenoxy]-benzyl}-piperazine-1-carboxylic acid benzo[d]isoxazol-3-ylamide

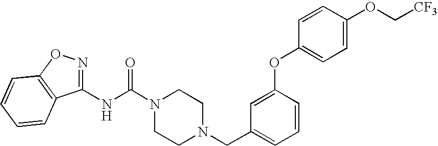

MS: 527.2. ¹H NMR (CDCl₃): 8.41 (s, 1H), 8.07 (d, J=8.0, 1H), 7.54-7.50 (m, 1H), 7.44 (d, J=8.5, 1H), 7.28 (d, J=8.0, 2H), 7.06 (d, J=7.5, 1H), 7.01-6.98 (m, 3H), 6.95-6.92 (m, 2H), 6.87-6.85 (m, 1H), 4.36-4.31 (m, 2H), 3.66-3.64 (m, 4H), 3.54 (s, 2H), 2.55-2.53 (m, 4H).

### Example 36

### 4-[3-(4-Chloro-phenoxy)-benzyl]-piperazine-1-carboxylic acid benzo[d]isoxazol-3-ylamide trifluoroacetic acid salt

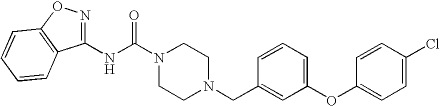

Step A: 4-(Benzo[d]isoxazol-3-ylcarbamoyl)-piperazine-1-carboxylic acid tert-butyl ester. A mixture of benzo[d]isoxazol-3-yl-carbamic acid phenyl ester (1.072 g) and piperazine-1-carboxylic acid tert-butyl ester (942 mg) in DMSO (8 mL) was stirred at 50° C. for 20 h, and then was diluted with water (400 mL), mixed thoroughly and filtered. The collected solid was dissolved in DCM and washed with water (×1) and satd. aq. NaHCO₃ (×1), dried and concentrated to give a brown solid. Purification by FCC gave 1.10 g (79%) of the product as a white crystalline solid.

Step B: Piperazine-1-carboxylic acid benzo[d]isoxazol-3-ylamide. A mixture of 4-(benzo[d]isoxazol-3-ylcarbamoyl)-piperazine-1-carboxylic acid tert-butyl ester (1.10 g) and TFA (2.5 mL) in DCM (32 mL) was stirred for 2.5 h and then concentrated to give 1.15 g (100%) of the title compound as a pale-yellow viscous oil. MS: 247.2.

Step C: 4-[3-(4-Chloro-phenoxy)-benzyl]-piperazine-1-carboxylic acid benzo[d]isoxazol-3-ylamide. A mixture of piperazine-1-carboxylic acid benzo[d]isoxazol-3-ylamide (60.5 mg), 3-(4-chlorophenoxy)-benzaldehyde (88.4 mg), Et₃N (0.1 mL), and MP—B(OAc)₃H (235 mg; resin loading=2.33 mmol/g) in THF (2.0 mL) was mixed on a shaker table for 19 h. The mixture was filtered and the filtrate was concentrated. The residue was purified by reverse-phase HPLC to give 44.6 mg (46%) of the title compound as the TFA salt. MS: 463.2. ¹H NMR (d₄-MeOH): 7.88 (d, J=7.8, 1H), 7.60-7.58 (m, 1H), 7.54-7.48 (m, 2H), 7.38-7.35 (m, 2H), 7.33-7.28 (m, 2H), 7.202-7.196 (m, 1H), 7.14-7.12 (dd, J=1.8, 8.4, 1H), 7.03-7.01 (m, 2H), 4.66-2.69 (br hump, 4H), 4.40 (s, 2H), 3.38 (br hump, 4H).

The compounds in Examples 37-57 were prepared using methods analogous to those described in Example 36.

### Example 37

### 4-[3-(4-Chloro-phenoxy)-benzyl]-piperazine-1-carboxylic acid benzo[1,2,5]thiadiazol-4-ylamide trifluoroacetic acid salt

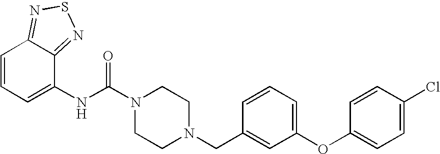

MS: 480.2. ¹H NMR (d₄-MeOH): 7.92-7.90 (dd, J=0.6, 7.2, 1H), 7.66-7.65 (d, J=9.0, 1H), 7.60-7.57 (dd, J=9.0, 7.8, 1H), 7.51-7.48 (t, J=8.4, 1H), 7.38-7.36 (m, 2H), 7.29 (br d, J=7.8, 1H), 7.21-7.20 (br m, 1H), 7.14-7.12 (dd, J=1.8, 7.8, 1H), 7.04-7.02 (m, 2H), 4.40 (s, 2H), 3.80-3.01 (br hump, 8H).

### Example 38

### 4-[3-(3,4-Dichloro-phenoxy)-benzyl]-piperazine-1-carboxylic acid benzo[1,2,5]thiadiazol-4-ylamide trifluoroacetic acid salt

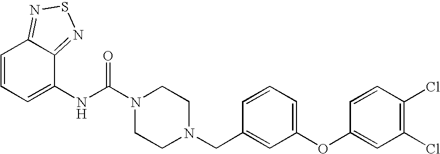

MS: 514.1. ¹H NMR (d₄-MeOH): 7.92-7.91 (dd, J=1.2, 7.8, 1H), 7.67-7.65 (dd, J=1.2, 9.0, 1H), 7.61-7.58 (dd, J=7.2, 9.0, 1H), 7.55-7.52 (t, J=7.8, 1H), 7.50 (d, J=9.0, 1H), 7.35 (d, J=7.2, 1H), 7.25 (br m, 1H), 7.21 (d, J=2.4, 1H), 7.20-7.18 (dd, J=2.4, 8.4, 1H), 6.99-6.97 (dd, J=3.0, 9.0, 1H), 4.58-4.22 (br hump, 4H), 4.42 (s, 2H), 3.72-3.01 (br hump, 4H).

### Example 39

### 4-[3-(3,5-Dichloro-phenoxy)-benzyl]-piperazine-1-carboxylic acid benzo[1,2,5]thiadiazol-4-ylamide trifluoroacetic acid salt

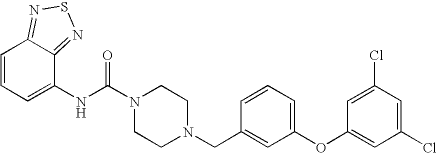

MS: 514.1. ¹H NMR (d₄-MeOH): 7.94-7.93 (dd, J=0.6, 7.2, 1H), 7.70-7.68 (dd, J=1.2, 9.0, 1H), 7.63-7.56 (m, 2H), 7.40 (d, J=7.8, 1H), 7.30-7.29 (m, 1H), 7.24-7.23 (m, 2H), 7.02 (d, J=1.8, 2H), 4.83-2.94 (br hump, 4H), 4.40 (s, 2H), 3.41 (br s, 4H).

### Example 40

### 4-[3-(3-Trifluoromethyl-phenoxy)-benzyl]-piperazine-1-carboxylic acid benzo[1,2,5]thiadiazol-4-ylamide trifluoroacetic acid salt

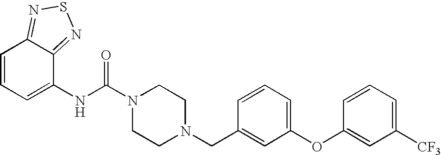

MS: 514.2. ¹H NMR (d₄-MeOH): 7.92-7.91 (dd, J=1.2, 7.2, 1H), 7.67-7.65 (dd, J=1.2, 9.0, 1H), 7.61-7.53 (m, 3H), 7.45 (d, J=7.8, 1H), 7.36 (d, J=7.8, 1H), 7.31-7.28 (m, 3H), 7.20-7.18 (m, 1H), 4.85-2.94 (br hump, 4H), 4.43 (s, 2H), 3.41 (br hump, 4H).

### Example 41

### 4-(3-Trifluoromethoxy-benzyl)-piperazine-1-carboxylic acid benzo[1,2,5]thiadiazol-4-ylamide trifluoroacetic acid salt

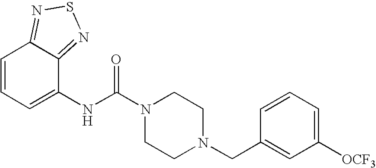

MS: 438.2. ¹H NMR (d₄-MeOH): 7.93-7.92 (dd, J=7.2, 0.6, 1H), 7.69-7.67 (1.2, 9.0, 1H), 7.64-7.60 (m, 2H), 7.57-7.55 (m, 2H), 7.47-7.45 (m, 1H), 4.48 (s, 2H), 4.33-3.66 (br hump, 4H), 3.42 (br s, 4H).

### Example 42

### 4-[3-(4-Chloro-phenoxy)-benzyl]-piperazine-1-carboxylic acid (6-chloro-pyridazin-3-yl)-amide trifluoroacetic acid salt

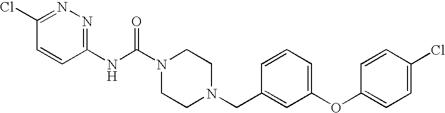

MS: 413.2. ¹H NMR (d₄-MeOH): 8.47 (s, 1H), 7.48 (t, J=8.4, 1H), 7.38-7.36 (m, 2H), 7.28 (d, J=7.2, 1H), 7.18 (t, J=1.8, 1H), 7.14-7.12 (dd, J=2.4, 7.8, 1H), 7.03-7.01 (m, 2H), 6.73 (s, 1H), 4.70-2.85 (br hump, 4H), 4.37 (s, 2H), 3.36 (br hump, 4H).

### Example 43

### 4-[3-(3,4-Dichloro-phenoxy)-benzyl]-piperazine-1-carboxylic acid (6-chloro-pyridazin-3-yl)-amide trifluoroacetic acid salt

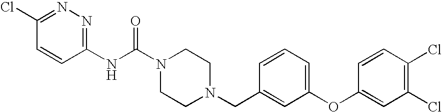

MS: 447.1. ¹H NMR (d₄-MeOH): 8.46 (s, 1H), 7.53-7.50 (s, 2H), 7.33 (d, J=7.8, 1H), 7.23 (t, J=1.8, 1H), 7.20 (d, J=3.0, 1H), 7.19-7.17 (m, 1H), 6.98-6.96 (dd, J=2.4, 8.4, 1H), 6.73 (s, 1H), 4.74-2.66 (br hump, 4H), 4.39 (s, 2H), 3.36 (br hump, 4H).

### Example 44

### 4-[3-(3-Trifluoromethyl-phenoxy)-benzyl]-piperazine-1-carboxylic acid (6-chloro-pyridazin-3-yl)-amide trifluoroacetic acid salt

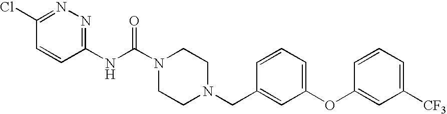

MS: 447.2. ¹H NMR (d₄-MeOH): 8.61 (br s, 1H), 7.57 (t, J=8.4, 1H), 7.53 (t, J=7.8, 1H), 7.45 (d, J=7.8, 1H), 7.34 (d, J=7.8, 1H), 7.29-7.25 (m, 3H), 7.18-7.17 (dd, J=1.8, 7.8, 1H), 6.84 (br s, 1H), 4.77-2.89 (br hump, 4H), 4.40 (s, 2H), 3.35 (br hump, 4H).

### Example 45

### 4-(4-Fluoro-3-phenoxy-benzyl)-piperazine-1-carboxylic acid (6-chloro-pyridazin-3-yl)-amide trifluoroacetic acid salt

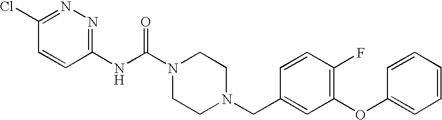

MS: 397.2. ¹H NMR (d₄-MeOH): 8.44 (d, J=1.8, 1H), 7.38-7.31 (m, 4H), 7.28-7.27 (dd, J=1.8, 7.8, 1H), 7.15-7.12 (m, 1H), 7.009-6.995 (m, 1H), 6.71 (d, J=1.8, 1H), 4.75-2.84 (br hump, 4H), 4.34 (s, 2H), 3.33 (br hump, 4H).

### Example 46

### 4-[3-(4-Chloro-phenoxy)-benzyl]-piperazine-1-carboxylic acid isoxazol-3-ylamide trifluoroacetic acid salt

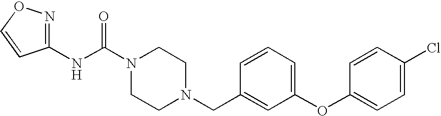

MS: 458.1. ¹H NMR (d₄-MeOH): 8.12 (d, J=9.0, 1H), 7.67 (d, J=9.6, 1H), 7.49 (t, J=7.8, 1H), 7.38-7.35 (m, 2H), 7.28 (d, J=7.8, 1H), 7.19 (t, J=1.8, 1H), 7.14-7.12 (dd, J=1.8, 7.8, 1H), 7.03-7.01 (m, 2H), 4.79-2.86 (br hump, 4H), 4.39 (s, 2H), 3.38 (br hump, 4H).

### Example 47

### 4-[3-(3,4-Dichloro-phenoxy)-benzyl]-piperazine-1-carboxylic acid isoxazol-3-ylamide trifluoroacetic acid salt

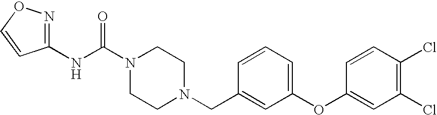

MS: 490.1. ¹H NMR (d₄-MeOH): 8.14 (d, J=9.6, 1H), 7.68 (d, J=9.0, 1H), 7.55-7.51 (m, 2H), 7.34 (d, J=7.8, 1H), 7.24 (t, J=2.4, 1H), 7.21 (d, J=2.4, 1H), 7.20-7.18 (m, 1H), 7.00-6.98 (dd, J=3.0, 9.0, 1H), 4.72-2.72 (br hump, 4H), 4.40 (s, 2H), 3.38 (br hump, 4H).

### Example 48

### 4-[3-(3-Trifluoromethyl-phenoxy)-benzyl]-piperazine-1-carboxylic acid isoxazol-3-ylamide trifluoroacetic acid salt

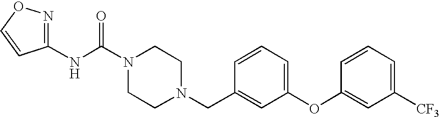

MS: 492.2. ¹H NMR (d₄-MeOH): 8.21 (br s, 1H), 7.72 (br s, 1H), 7.59-7.53 (m, 2H), 7.45 (d, J=7.8, 1H), 7.35 (d, J=7.8, 1H), 7.30-7.27 (m, 3H), 7.20-7.18 (dd, J=1.8, 7.8, 1H), 4.70-2.82 (br hump, 4H), 4.41 (s, 2H), 3.39 (br hump, 4H).

### Example 49

### 4-(4-Fluoro-3-phenoxy-benzyl)-piperazine-1-carboxylic acid isoxazol-3-ylamide trifluoroacetic acid salt

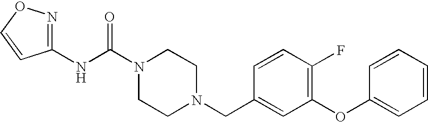

MS: 442.2. ¹H NMR (d₄-MeOH): 8.13 (br s, 1H), 7.67 (d, J=9.0, 1H), 7.39-7.32 (m, 4H), 7.30-7.28 (dd, J=2.4, 7.8, 1H), 7.15-7.12 (m, 1H), 7.02-7.00 (dd, J=1.2, 9.0, 1H), 4.76-2.55 (br hump, 4H), 4.35 (s, 2H), 3.34 (br hump, 4H).

### Example 50

### 4-[3-(3,4-Dichloro-phenoxy)-benzyl]-piperazine-1-carboxylic acid benzo[d]isoxazol-3-ylamide trifluoroacetic acid salt

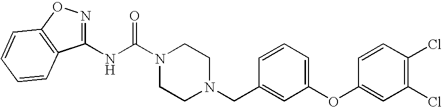

MS: 497.1. ¹H NMR (d₄-MeOH): 7.87 (d, J=2.4, 1H), 7.61-7.59 (m, 1H), 7.55-7.50 (m, 3H), 7.35-7.30 (m, 2H), 7.25-7.24 (m, 1H), 7.21 (d, J=3.0, 1H), 7.20-7.18 (m, 1H), 6.99-6.97 (dd, J=2.4, 8.4, 1H), 4.55-2.93 (br hump, 4H), 4.42 (s, 2H), 3.40 (br hump, 4H).

### Example 51

### 4-[3-(3-Trifluoromethyl-phenoxy)-benzyl]-piperazine-1-carboxylic acid benzo[d]isoxazol-3-ylamide trifluoroacetic acid salt

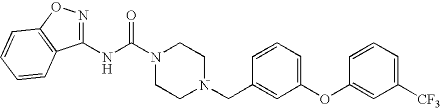

MS: 497.2. ¹H NMR (d₄-MeOH): 7.88 (d, J=7.8, 1H), 7.60-7.52 (m, 4H), 7.45 (d, J=7.8, 1H), 7.35 (d, J=7.8, 1H), 7.32-7.27 (m, 4H), 7.19-7.17 (dd, J=2.4, 7.8, 1H), 4.69-2.98 (br hump, 4H), 4.42 (s, 2H), 3.40 (br hump, 4H).

### Example 52

### 4-(3-Trifluoromethoxy-benzyl)-piperazine-1-carboxylic acid benzo[d]isoxazol-3-ylamide trifluoroacetic acid salt

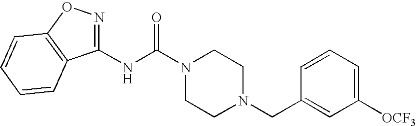

MS: 421.2. ¹H NMR (d₄-MeOH): 7.88 (d, J=7.8, 1H), 7.63-7.58 (m, 2H), 7.55-7.50 (m, 3H), 7.43 (d, J=7.2, 1H), 7.32 (t, J=7.8, 1H), 4.61-2.99 (br hump, 4H), 4.47 (s, 2H), 3.42 (br hump, 4H).

### Example 53

### 4-[3-(4-Chloro-phenoxy)-benzyl]-piperazine-1-carboxylic acid (1H-pyrazol-3-yl)-amide trifluoroacetic acid salt

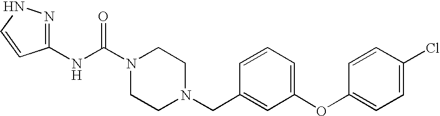

MS: 412.2. ¹H NMR (d₄-MeOH): 7.89 (br hump, 1H), 7.50 (t, J=7.8 Hz, 1H), 7.39-7.36 (m, 2H), 7.28 (d, J=7.8, 1H), 7.19 (t, J=1.8, 1H), 7.15-7.13 (dd, J=1.8, 7.8, 1H), 7.04-7.02 (m, 2H), 6.38 (br hump, 1H), 4.50-2.95 (br hump, 4H), 3.35 (br hump, 4H).

### Example 54

### 4-[3-(3,4-Dichloro-phenoxy)-benzyl]-piperazine-1-carboxylic acid (1H-pyrazol-3-yl)-amide trifluoroacetic acid salt

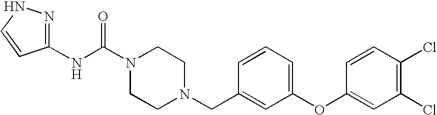

MS: 446.1. ¹H NMR (d₄-MeOH): 7.91 (br hump, 1H), 7.55-7.52 (m, 2H), 7.34 (d, J=7.8, 1H), 7.25 (t, J=1.8, 1H), 7.22 (d, J=3.0, 1H), 7.20-7.18 (dd, J=2.4, 7.8, 1H), 7.01-6.99 (dd, J=3.0, 9.0, 1H), 6.44 (br hump, 1H), 4.6-2.85 (br hump, 4H), 4.40 (s, 2H), 3.37 (br hump, 4H).

### Example 55

### 4-[3-(3-Trifluoromethyl-phenoxy)-benzyl]-piperazine-1-carboxylic acid (1H-pyrazol-3-yl)-amide trifluoroacetic acid salt

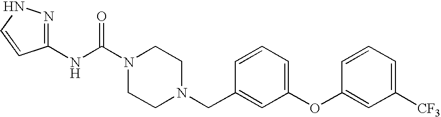

MS: 446.2. ¹H NMR (d₄-MeOH): 7.98 (br hump, 1H), 7.61-7.55 (m, 2H), 7.47 (d, J=7.8, 1H), 7.36 (d, J=7.8, 1H), 7.31-7.27 (m, 3H), 7.22-7.20 (dd, J=2.4, 7.8, 1H), 4.60-2.89 (br hump, 4H), 4.41 (s, 2H), 3.36 (br hump, 4H).

### Example 56

### 4-(4-Fluoro-3-phenoxy-benzyl)-piperazine-1-carboxylic acid (1H-pyrazol-3-yl)-amide trifluoroacetic acid salt

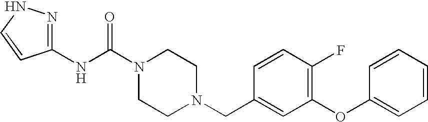

MS: 396.2. ¹H NMR (d₄-MeOH): 7.89 (br hump, 1H), 7.41-7.33 (m, 4H), 7.31-7.29 (dd, J=1.8, 7.8, 1H), 7.16 (t, J=7.2, 1H), 7.03 (d, J=7.8, 1H), 6.43 (br hump, 1H), 4.60-2.71 (br hump, 4H), 4.35 (s, 2H), 3.35 (br hump, 4H).

### Example 57

### 4-[3-(4-Chloro-phenoxy)-benzyl]-piperazine-1-carboxylic acid (1H-tetrazol-5-yl)-amide trifluoroacetic acid salt

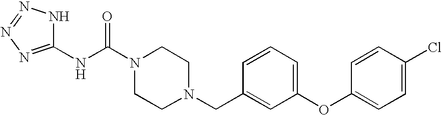

MS: 414.2. ¹H NMR (d₄-MeOH): 7.51 (t, J=7.8, 1H), 7.40-7.39 (m, 2H), 7.28 (d, J=7.8, 1H), 7.19 (br s, 1H), 7.16-7.14 (dd, J=1.8, 7.8, 1H), 7.05-7.03 (m, 2H), 4.36 (s, 2H), 4.10-3.60 (br hump, 4H), 3.44-3.21 (br hump, 4H).

### Example 58

### 4-[3-(4-Chloro-phenoxy)-benzyl]-piperazine-1-carboxylic acid pyrazin-2-ylamide trifluoroacetic acid salt

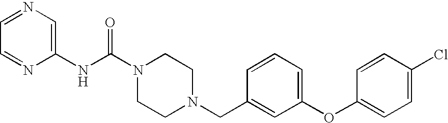

Step A: 4-(Pyrazin-2-ylcarbamoyl)-piperazine-1-carboxylic acid tert-butyl ester. To a mixture of aminopyrazine (530 mg) in DCM (52 mL) was added di(N-succinimidyl)carbonate (1.43 g). The heterogeneous mixture was stirred for 21 h and then was treated with N-Boc-piperazine (1.62 g). After 8 h, the mixture was concentrated and the residue purified by FCC (NH₃/MeOH/DCM) to give 1.07 g (63%) of the title compound as a white solid. MS: 308.2.

Step B: Piperazine-1-carboxylic acid pyrazin-2-ylamide. The title compound was prepared using methods analogous to those described in Example 36, Step B. MS: 208.2.

Step C: 4-[3-(4-Chloro-phenoxy)-benzyl]-piperazine-1-carboxylic acid pyrazin-2-ylamide. The title compound was prepared a the TFA salt using methods analogous to those described in Example 36, Step C.

MS: 424.2. ¹H NMR (d₄-MeOH): 9.18 (br s, 1H), 8.34 (br s, 2H), 7.50 (t, J=7.8, 1H), 7.39-7.36 (m, 2H), 7.29 (d, J=7.2, 1H), 7.201-7.195 (m, 1H), 7.15-7.13 (m, 1H), 7.04-7.02 (m, 2H), 4.58-2.83 (br hump, 4H), 4.38 (s, 2H), 3.37 (br hump, 4H).

The compounds in Examples 59-61 were prepared using methods analogous to those described in Example 58.

### Example 59

### 4-[3-(3,4-Dichloro-phenoxy)-benzyl]-piperazine-1-carboxylic acid pyrazin-2-ylamide trifluoroacetic acid salt

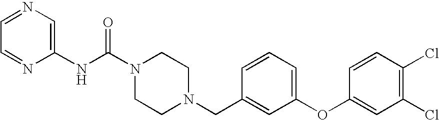

MS: 458.1. ¹H NMR (d₄-MeOH): 9.06 (s, 1H), 8.31 (s, 1H), 8.21 (s, 1H), 7.55-7.51 (m, 2H), 7.34 (d, 1H), 7.24-7.18 (m, 3H), 7.00-6.98 (dd, J=2.4, 8.4, 1H), 4.57-2.85 (br hump, 4H), 4.40 (s, 2H), 3.36 (br hump, 4H).

### Example 60

### 4-[3-(3-Trifluoromethyl-phenoxy)-benzyl]-piperazine-1-carboxylic acid pyrazin-2-ylamide trifluoroacetic acid salt

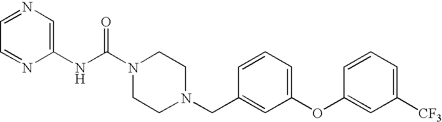

MS: 458.2. ¹H NMR (d₄-MeOH): 9.22 (br s, 1H), 8.36 (br s, 2H), 7.59-7.53 (m, 2H), 7.45 (d, J=7.8, 1H), 7.35 (d, J=7.8, 1H), 7.30-7.26 (m, 3H), 7.20-7.18 (m, 1H), 4.80-2.94 (br hump, 4H), 4.41 (s, 2H), 3.37 (br hump, 4H).

### Example 61

### 4-(3-Trifluoromethoxy-benzyl)-piperazine-1-carboxylic acid pyrazin-2-ylamide trifluoroacetic acid salt

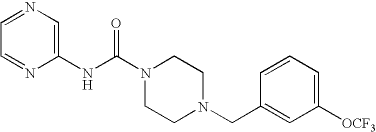

MS: 382.2. ¹H NMR (d₄-MeOH): 9.06 (s, 1H), 8.32 (s, 1H), 8.21 (s, 1H), 7.61 (t, J=7.8, 1H), 7.56-7.53 (m, 2H), 7.45-7.44 (m, 1H), 4.62-2.89 (br hump, 4H), 4.46 (s, 2H), 3.38 (br hump, 4H).

### Example 62

### N-1,2-Benzisoxazol-3-yl-4-[(2,2-difluoro-1,3-benzodioxol-5-yl)methyl]piperidine-1-carboxamide

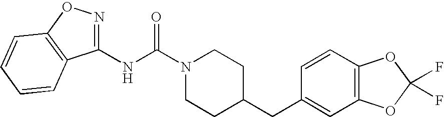

Step A: 4-(2,2-Difluoro-benzo[1,3]dioxol-5-ylmethyl)-piperidine-1-carboxylic acid tert-butyl ester. 1-Boc-4-methylene piperidine (454.6 mg) was degassed (neat) for 15 min, and then treated with a THF solution of 9-borabicyclo[3.3.1]nonane (BBN; 0.5 M in THF, 4.7 mL). The reaction mixture was refluxed for 3.5 h, then cooled to rt. The reaction mixture was then added, via cannula, to a preformed solution consisting of 5-bromo-2,2-difluoro-1,3-benzodioxole (502.3 mg), [1,1′-bis(diphenylphosphino)ferrocene]dichloropalladium(II) (Pd(dppf)Cl₂), complex with DCM (45.9 mg), and potassium carbonate (369.6 mg) in DMF/H₂O (10 mL/1 mL). The resultant mixture was heated at 60° C. for 18 h, cooled to rt, poured into water, basified to pH 11 with 10% NaOH, and extracted with EtOAc (3×). The organic layers were combined, dried (Na₂SO₄), and concentrated. The crude residue was purified (FCC) to give 4-(2,2-difluoro-benzo[1,3]dioxol-5-ylmethyl)-piperidine-1-carboxylic acid tert-butyl ester (608.2 mg, 81%).

Step B: 4-(2,2-Difluoro-benzo[1,3]dioxol-5-ylmethyl)-piperidine. The title compound was prepared using methods analogous to those described in Example 9, Step D.

Step C: N-1,2-Benzisoxazol-3-yl-4-[(2,2-difluoro-1,3-benzodioxol-5-yl)methyl]piperidine-1-carboxamide. The title compound was prepared using methods analogous to those described in Example 1, Step C. MS: 414.4. ¹H NMR (d₄-MeOH): 7.83-7.80 (m, 1H), 7.59-7.54 (m, 1H), 7.53-7.50 (m, 1H), 7.31-7.27 (m, 1H), 7.11-7.07 (m, 2H), 7.00-6.97 (m, 1H), 4.24-4.17 (m, 2H), 2.98-2.90 (m, 2H), 2.64 (d, J=7.2, 2H), 1.89-1.81 (m, 1H), 1.75-1.70 (m, 2H), 1.33-1.24 (m, 2H).

### Example 63

### 4-(3-o-Tolylethynyl-benzyl)-piperazine-1-carboxylic acid benzo[d]isoxazol-3-ylamide

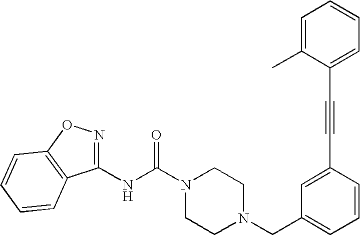

Step A: 1-(3-Iodo-benzyl)-piperazine. The title compound was prepared using methods analogous to those described in Example 1, Step B. MS: 403.1.

Step B: 4-(3-Iodo-benzyl)-piperazine-1-carboxylic acid benzo[d]isoxazol-3-ylamide. The title compound was prepared using methods analogous to those described in Example 1, Step C. MS: 463.1.

Step C: 4-(3-o-Tolylethynyl-benzyl)-piperazine-1-carboxylic acid benzo[d]isoxazol-3-ylamide. To a solution of Pd(PPh₃)₂Cl₂ (7.2 mg) in THF/Et₃N (1 mL each) was added 4-(3-iodo-benzyl)-piperazine-1-carboxylic acid benzo[d]isoxazol-3-ylamide (100.0 mg). The solution was degassed for 15 min, then copper(I) iodide (4.6 mg) and 2-ethynyltoluene (37.8 mg) were added. The reaction mixture was stirred at rt for 10 min, then poured into water and extracted with EtOAc (3×). The organic layers were combined, washed with NH₄OH, dried (Na₂SO₄), and concentrated. The crude residue was purified (FCC) to afford the title compound (89.3 mg, 92%). MS: 451.2. ¹H NMR (d₄-MeOH): 7.88-7.83 (m, 1H), 7.62-7.51 (m, 3H), 7.50-7.44 (m, 2H), 7.42-7.36 (m, 2H), 7.34-7.24 (m, 3H), 7.22-7.17 (m, 1H), 3.71-3.60 (m, 6H), 2.61-2.55 (m, 4H), 2.53 (s, 3H).

The compounds in Examples 64-80 were prepared using methods analogous to those described in Example 63.

### Example 64

### N-1,2-Benzisoxazol-3-yl-4-(3-{[2-(trifluoromethyl)-phenyl]-ethynyl}benzyl)-piperazine-1-carboxamide

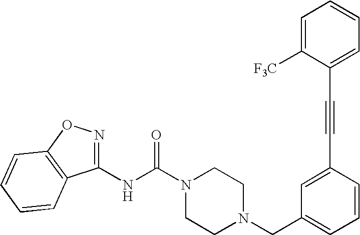

MS: 505.2. ¹H NMR (d₄-MeOH): 7.86 (d, J=8.1, 1H), 7.78-7.72 (m, 2H), 7.67-7.62 (m, 1H), 7.61-7.52 (m, 4H), 7.49-7.39 (m, 3H), 7.34-7.29 (m, 1H), 3.71-3.60 (m, 6H), 2.62-2.52 (m, 4H).

### Example 65

### N-1,2-Benzisoxazol-3-yl-4-{3-[(2-methoxyphenyl)-ethynyl]-benzyl}-piperazine-1-carboxamide

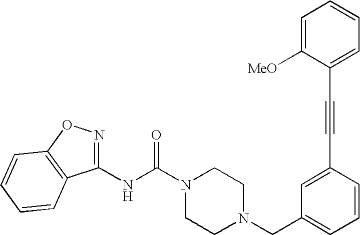

MS: 467.2. ¹H NMR (d₄-MeOH): 7.88-7.83 (m, 1H), 7.61-7.51 (m, 3H), 7.47-7.42 (m, 2H), 7.39-7.29 (m, 4H), 7.04 (d, J=8.4, 1H), 6.98-6.93 (m, 1H), 3.93 (s, 3H), 3.69-3.64 (m, 4H), 3.62 (s, 2H), 2.61-2.55 (m, 4H).

### Example 66

### N-1,2-Benzisoxazol-3-yl-4-{3-[(2-fluorophenyl)ethynyl]-benzyl}-piperazine-1-carboxamide

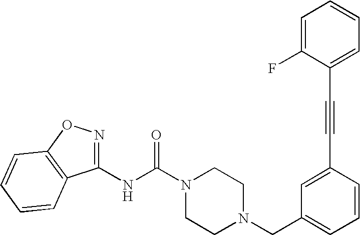

MS: 455.2. ¹H NMR (d₄-MeOH): 7.88-7.83 (m, 1H), 7.62-7.51 (m, 4H), 7.50-7.46 (m, 1H), 7.45-7.37 (m, 3H), 7.34-7.29 (m, 1H), 7.24-7.15 (m, 2H), 3.71-3.59 (m, 6H), 2.61-2.54 (m, 4H).

### Example 67

### N-1,2-Benzisoxazol-3-yl-4-{3-[(2-bromophenyl)-ethynyl]benzyl}-piperazine-1-carboxamide

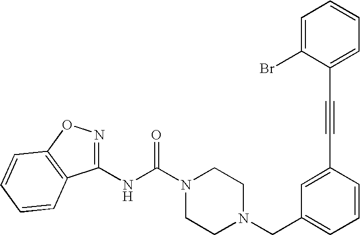

MS: 515.1. ¹H NMR (CDCl₃): 8.11-8.07 (m, 1H), 7.66-7.63 (m, 1H), 7.60-7.57 (m, 2H), 7.56-7.51 (m, 2H), 7.50-7.47 (m, 1 H), 7.42-7.28 (m, 5H), 7.23-7.19 (m, 1H), 3.66-3.62 (m, 4H), 3.60 (s, 2H), 2.60-2.55 (m, 4H).

### Example 68

### 4-(3-Ethynyl-benzyl)-piperazine-1-carboxylic acid benzo[d]isoxazol-3-ylamide

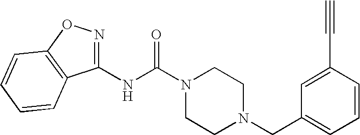

Step A: 4-(3-Trimethylsilanylethynyl-benzyl)-piperazine-1-carboxylic acid benzo[d]isoxazol-3-ylamide. The title compound was prepared using methods analogous to those described in Example 63, Step C. MS: 433.2.

Step B: 4-(3-Ethynyl-benzyl)-piperazine-1-carboxylic acid benzo[d]isoxazol-3-ylamide. To a solution of 4-(3-trimethylsilanylethynyl-benzyl)-piperazine-1-carboxylic acid benzo[d]isoxazol-3-ylamide (396.2 mg) in MeOH (10 mL) was added potassium carbonate (500 mg). The reaction mixture was stirred at rt for 2 h, then filtered through diatomaceous earth and concentrated. The crude residue was purified (FCC) to give the title compound (291.7 mg, 88%). MS: 361.2. ¹H NMR (CDCl₃): 8.11-8.08 (m, 1H), 7.88 (s, 1H), 7.57-7.50 (m, 2H), 7.49-7.46 (m, 1H), 7.45-7.42 (m, 1H), 7.37-7.29 (m, 3H), 3.68-3.62 (m, 4H), 3.56 (s, 2H), 3.10 (s, 1H), 2.58-2.53 (m, 4H).

### Example 69

### N-1,2-Benzisoxazol-3-yl-4-{3-[3-(dimethylamino)prop-1-yn-1-yl]benzyl}-piperazine-1-carboxamide

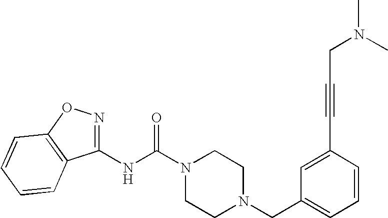

MS: 418.2. ¹H NMR (d₄-MeOH): 7.87-7.83 (m, 1H), 7.61-7.56 (m, 1H), 7.56-7.52 (m, 1H), 7.49-7.46 (m, 1H), 7.39-7.29 (m, 4H), 3.67-3.62 (m, 4H), 3.59 (s, 2H), 3.51 (s, 2H), 2.58-2.51 (m, 4H), 2.40 (s, 6H).

### Example 70

### N-1,2-Benzisoxazol-3-yl-4-[3-(cyclohexylethynyl)benzyl]-piperazine-1-carboxamide

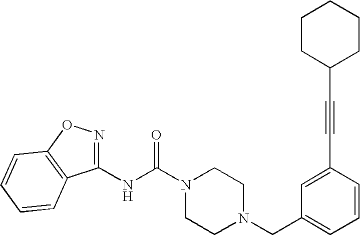

MS: 443.2. ¹H NMR (d₆-DMSO): 8.00-7.96 (m, 1H), 7.60-7.55 (m, 1H), 7.53-7.51 (m, 1H), 7.41-7.38 (m, 1H), 7.34-7.26 (m, 4H), 3.70-3.64 (m, 4H), 3.55 (s, 2H), 2.65-2.60 (m, 1H), 2.54-2.49 (m, 4H), 1.91-1.85 (m, 2H), 1.79-1.72 (m, 2H), 1.58-1.48 (m, 3H), 1.43-1.35 (m, 3H).

### Example 71

### N-1,2-Benzisoxazol-3-yl-4-[3-(cyclopentylethynyl)benzyl]-piperazine-1-carboxamide

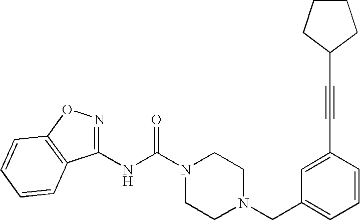

MS: 429.2. ¹H NMR (d₄-MeOH): 7.87-7.83 (m, 1H), 7.61-7.57 (m, 1H), 7.55-7.52 (m, 1H), 7.40-7.37 (m, 1H), 7.34-7.26 (m, 4H), 3.68-3.62 (m, 4H), 3.57 (s, 2H), 2.90-2.83 (m, 1H), 2.57-2.52 (m, 4H), 2.07-1.98 (m, 2H), 1.84-1.77 (m, 2H), 1.74-1.62 (m, 4H).

### Example 72

### N-1,2-Benzisoxazol-3-yl-4-{3-[(2-chlorophenyl)-ethynyl]benzyl}-piperazine-1-carboxamide

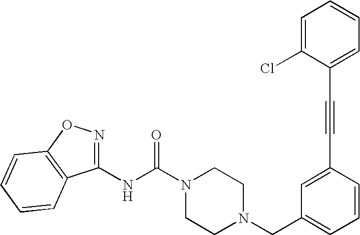

MS: 471.2. ¹H NMR (d₄-MeOH): 7.87-7.84 (m, 1H), 7.63-7.56 (m, 3H), 7.55-7.47 (m, 3H), 7.45-7.29 (m, 5H), 3.69-3.61 (m, 6H), 2.61-2.55 (m, 4H).

### Example 73

### N-1,2-Benzisoxazol-3-yl-4-{3-[(3-chlorophenyl)ethynyl]benzyl}-piperazine-1-carboxamide

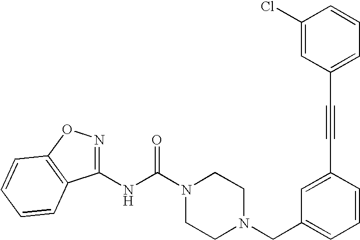

MS: 471.2. ¹H NMR (CDCl₃): 8.12-8.08 (m, 1H), 7.69 (s, 1H), 7.57-7.51 (m, 3H), 7.50-7.45 (m, 2H), 7.45-7.42 (m, 1H), 7.37-7.28 (m, 5H), 3.68-3.63 (m, 4H), 3.59 (s, 2H), 2.60-2.54 (m, 4H).

### Example 74

### N-1,2-Benzisoxazol-3-yl-4-{3-[(4-chlorophenyl)ethynyl]benzyl}-piperazine-1-carboxamide

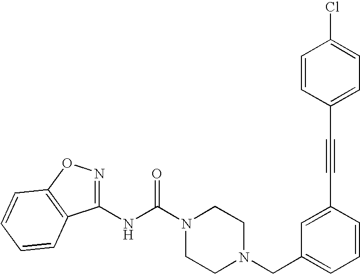

MS: 471.2. ¹H NMR (CDCl₃): 8.11-8.08 (m, 1H), 7.76 (s, 1H), 7.57-7.51 (m, 2H), 7.50-7.45 (m, 4H), 7.38-7.33 (m, 4H), 7.31-7.28 (m, 1H), 3.68-3.63 (m, 4H), 3.59 (s, 2H), 2.61-2.52 (m, 4H).

### Example 75

### N-1,2-Benzisoxazol-3-yl-4-{3-[(3,4-dichlorophenyl)ethynyl]-benzyl}-piperazine-1-carboxamide

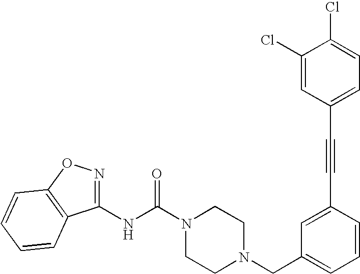

MS: 505.1. ¹H NMR (CDCl₃): 8.11-8.08 (m, 1H), 7.69 (s, 1H), 7.65 (d, J=1.9, 1H), 7.56-7.52 (m, 2H), 7.49-7.43 (m, 3H), 7.39-7.35 (m, 3H), 7.32-7.28 (m, 1H), 3.67-3.64 (m, 4H), 3.59 (s, 2H), 2.60-2.55 (m, 4H).

### Example 76

### N-1,2-Benzisoxazol-3-yl-4-[3-(cyclopropylethynyl)benzyl]-piperazine-1-carboxamide

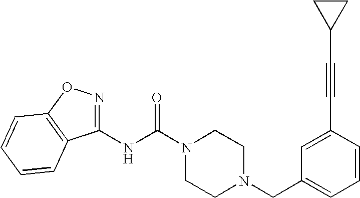

MS: 401.2. ¹H NMR (CDCl₃): 8.09 (d, J=8.1, 1H), 7.57-7.51 (m, 1H), 7.49-7.46 (m, 1H), 7.40-7.38 (m, 1H), 7.33-7.23 (m, 5H), 3.66-3.60 (m, 4H), 3.53 (s, 2H), 2.57-2.51 (m, 4H), 1.51-1.44 (m, 1H), 0.92-0.87 (m, 2H), 0.85-0.81 (m, 2H).

### Example 77

### N-1,2-Benzisoxazol-3-yl-4-[3-(thiophen-3-ylethynyl)benzyl]-piperazine-1-carboxamide

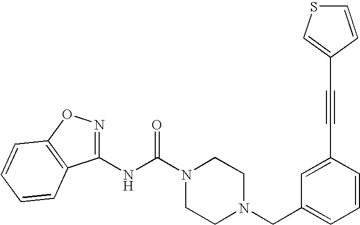

MS: 443.2. ¹H NMR (d₄-MeOH): 7.86-7.81 (m, 1H), 7.64-7.49 (m, 4H), 7.46-7.27 (m, 5H), 7.21-7.16 (m, 1H), 3.69-3.56 (m, 6H), 2.60-2.50 (m, 4H).

### Example 78

### 4-{3-[(2-Chlorophenyl)ethynyl]benzyl}-N-pyrazin-2-ylpiperazine-1-carboxamide hydrochloride salt

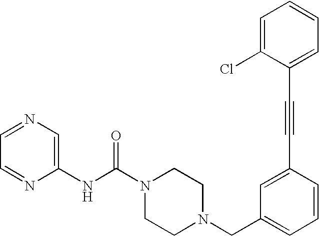

MS: 431.2. ¹H NMR (d₆-DMSO): 9.83-9.82 (m, 1H), 9.03-9.02 (m, 1H), 8.34-8.32 (m, 1H), 8.25 (d, J=2.6, 1H), 7.84-7.82 (m, 1H), 7.71-7.65 (m, 3H), 7.64-7.61 (m, 1H), 7.60-7.55 (m, 1H), 7.50-7.41 (m, 2H), 4.43-4.39 (m, 2H), 4.32-4.25 (m, 2H), 3.41-3.25 (m, 4H), 3.14-3.03 (m, 2H).

### Example 79

### 4-{3-[(2-Chlorophenyl)ethynyl]benzyl}-N-pyridazin-3-ylpiperazine-1-carboxamide

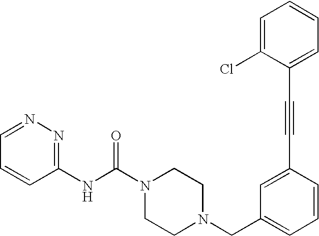

MS: 432.2. ¹H NMR (d₄-MeOH): 8.82-8.76 (m, 1H), 8.15-8.08 (m, 1H), 7.62-7.56 (m, 3H), 7.50-7.46 (m, 2H), 7.43-7.29 (m, 4H), 3.67-3.58 (m, 6H), 2.60-2.50 (m, 4H).

### Example 80

### 4-{3-[(2-Chlorophenyl)ethynyl]benzyl}-N-(5-methylpyrazin-2-yl)piperazine-1-carboxamide

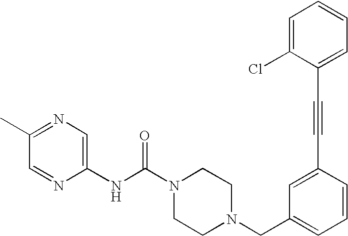

MS: 446.2. ¹H NMR (d₄-MeOH): 8.89 (d, J=1.5, 1H), 8.19-8.16 (m, 1H), 7.61-7.56 (m, 2H), 7.50-7.45 (m, 2H), 7.42-7.29 (m, 4H), 3.62-3.56 (m, 6H), 2.55-2.50 (m, 4H), 2.46 (s, 3H).

### Example 81

### N-1,2-Benzisoxazol-3-yl-4-{3-[(2,4-dichlorophenyl)-ethynyl]benzyl}-piperazine-1-carboxamide

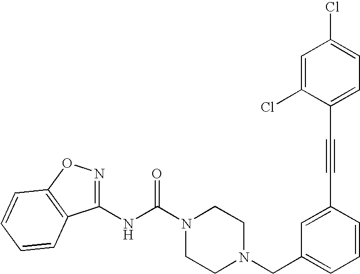

To a solution of Pd(PPh₃)₂Cl₂ (7.2 mg) in THF/Et₃N (1 mL each) was added 1,3-dichloro-4-iodobenzene (60.3 mg). The solution was degassed for 15 min, then copper(I) iodide (4.3 mg) and 4-(3-ethynyl-benzyl)-piperazine-1-carboxylic acid benzo[d]isoxazol-3-ylamide (75.6 mg) were added. The reaction mixture was stirred at rt for 3 h, then poured into water, and extracted with EtOAc (3×). The organic layers were combined, washed with NH₄OH, dried (Na₂SO₄), and concentrated. The crude residue was purified (FCC) to give the title compound (70.8 mg, 67%). MS: 505.1. ¹H NMR (d₄-MeOH): 7.87-7.83 (m, 1H), 7.61-7.56 (m, 4H), 7.55-7.52 (m, 1H), 7.51-7.47 (m, 1H), 7.46-7.36 (m, 3H), 7.34-7.29 (m, 1H), 3.69-3.60 (m, 6H), 2.61-2.53 (m, 4).

The compounds in Examples 82-93 were prepared using methods analogous to those described in Example 81.

### Example 82

### N-1,2-Benzisoxazol-3-yl-4-(3-{[2-(trifluoromethoxy)phenyl]-ethynyl}benzyl)-piperazine-1-carboxamide

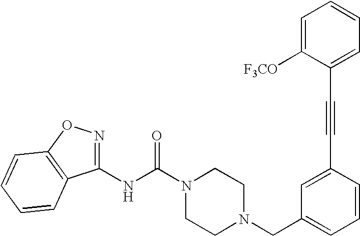

MS: 521.2. ¹H NMR (CDCl₃): 8.12-8.08 (m, 1H), 7.64-7.59 (m, 1H), 7.57-7.52 (m, 2H), 7.51-7.47 (m, 2H), 7.43-7.35 (m, 4H), 7.34-7.29 (m, 3H), 3.66-3.61 (m, 4H), 3.60 (s, 2H), 2.62-2.53 (m, 4H).

### Example 83

### N-1,2-Benzisoxazol-3-yl-4-{3-[(3,5-dichlorophenyl)ethynyl]benzyl}-piperazine-1-carboxamide

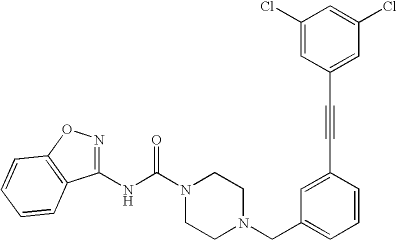

MS: 505.1. ¹H NMR (d₄-MeOH): 7.87-7.83 (m, 1H), 7.62-7.56 (m, 2H), 7.56-7.52 (m, 1H), 7.52-7.47 (m, 4H), 7.46-7.43 (m, 1H), 7.43-7.38 (m, 1H), 7.34-7.29 (m, 1H), 3.69-3.64 (m, 4H), 3.63 (s, 2H), 2.60-2.54 (m, 4H).

### Example 84

### N-1,2-Benzisoxazol-3-yl-4-{3-[(2,5-dichlorophenyl)ethynyl]benzyl}-piperazine-1-carboxamide

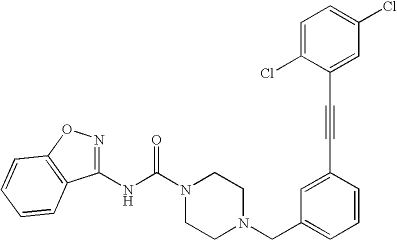

MS: 505.1. ¹H NMR (CDCl₃): 8.11-8.08 (m, 1H), 7.59-7.56 (m, 2H), 7.56-7.47 (m, 4H), 7.40-7.36 (m, 3H), 7.32-7.24 (m, 2H), 3.67-3.62 (m, 4H), 3.60 (s, 2H), 2.62-2.55 (m, 4H).

### Example 85

### N-1,2-Benzisoxazol-3-yl-4-{3-[(2-cyanophenyl)ethynyl]benzyl}-piperazine-1-carboxamide

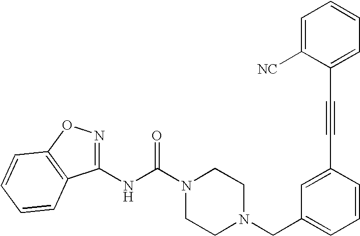

MS: 462.2. ¹H NMR (d₄-MeOH): 7.87-7.84 (m, 1H), 7.82-7.79 (m, 1H), 7.75-7.68 (m, 2H), 7.67-7.63 (m, 1H), 7.61-7.51 (m, 4H), 7.49-7.41 (m, 2H), 7.34-7.29 (m, 1H), 3.70-3.60 (m, 6H), 2.63-2.52 (m, 4H).

### Example 86

### N-1,2-Benzisoxazol-3-yl-4-[3-(naphthalen-1-ylethynyl)benzyl]piperazine-1-carboxamide

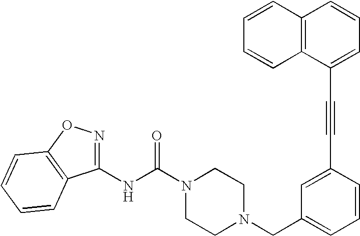

MS: 487.2. ¹H NMR (CDCl₃): 8.48-8.43 (m, 1H), 8.10-8.06 (m, 1H), 7.90-7.84 (m, 2H), 7.79-7.76 (m, 1H), 7.66-7.44 (m, 8H), 7.42-7.36 (m, 2H), 7.30-7.27 (m, 1H), 3.67-3.59 (m, 6H), 2.62-2.54 (m, 4H).

### Example 87

### Methyl 2-[(3-{[4-(1,2-benzisoxazol-3-ylcarbamoyl)piperazin-1-yl]methyl}phenyl)ethynyl]benzoate

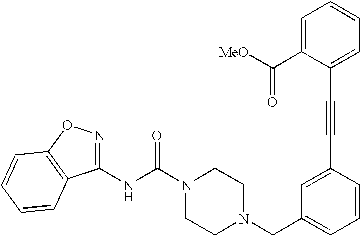

MS: 495.2. ¹H NMR (d₄-MeOH): 7.99-7.95 (m, 1H), 7.87-7.84 (m, 1H), 7.70-7.66 (m, 1H), 7.62-7.57 (m, 3H), 7.56-7.45 (m, 3H), 7.44-7.38 (m, 2H), 7.34-7.30 (m, 1H), 3.98 (s, 3H), 3.69-3.62 (m, 6H), 2.61-2.56 (m, 4H).

### Example 88

### N-1,2-Benzisoxazol-3-yl-4-{3-[(3-cyanophenyl)ethynyl]benzyl}piperazine-1-carboxamide

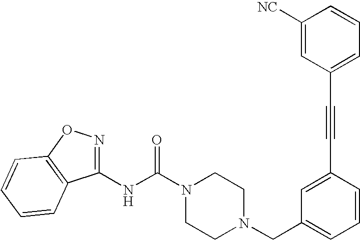

MS: 462.2. ¹H NMR (d₄-MeOH): 7.90-7.88 (m, 1H), 7.85-7.80 (m, 2H), 7.74-7.71 (m, 1H), 7.60-7.55 (m, 3H), 7.52 (d, J=8.5, 1H), 7.49-7.47 (m, 1H), 7.44-7.42 (m, 1H), 7.39 (t, J=7.6, 1H), 7.30 (t, J=7.5, 1H), 3.66-3.63 (m, 4H), 3.62 (s, 2H), 2.58-2.54 (m, 4H).

### Example 89

### N-1,2-Benzisoxazol-3-yl-4-[3-(1,3-benzodioxol-5-ylethynyl)benzyl]-piperazine-1-carboxamide

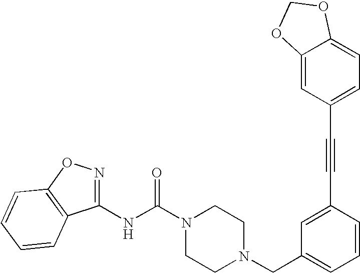

MS: 481.2. ¹H NMR (d₄-MeOH): 7.88-7.84 (m, 1H), 7.63-7.53 (m, 3H), 7.45-7.30 (m, 4H), 7.09-7.05 (dd, J=8.0, 1.6, 1H), 6.99-6.98 (m, 1H), 6.87-6.84 (m, 1H), 6.01 (s, 2H), 3.69-3.65 (m, 4H), 3.62 (s, 2H), 2.61-2.55 (m, 4H).

### Example 90

### N-1,2-Benzisoxazol-3-yl-4-{3-[(2,3-dichlorophenyl)ethynyl]benzyl}-piperazine-1-carboxamide

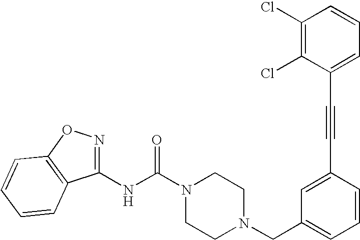

MS: 505.1. ¹H NMR (d₄-MeOH): 7.87-7.84 (m, 1H), 7.64-7.50 (m, 6H), 7.48-7.40 (m, 2H), 7.36-7.30 (m, 2H), 3.71-3.62 (m, 6H), 2.63-2.54 (m, 4H).

### Example 91

### N-1,2-Benzisoxazol-3-yl-4-{3-[(2-cyano-3-fluorophenyl)ethynyl]-benzyl}piperazine-1-carboxamide

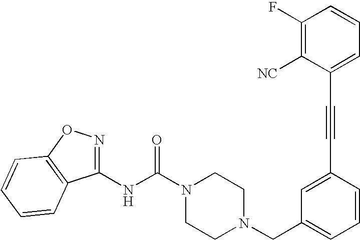

MS: 480.2. ¹H NMR (d₄-MeOH): 7.85-7.82 (m, 1H), 7.76-7.69 (m, 1H), 7.67-7.64 (m, 1H), 7.60-7.35 (m, 7H), 7.33-7.27 (m, 1H), 3.68-3.62 (m, 6H), 2.61-2.53 (m, 4H).

### Example 92

### N-1,2-Benzisoxazol-3-yl-4-(3-{[2-(cyanomethyl)phenyl]ethynyl}-benzyl)piperazine-1-carboxamide

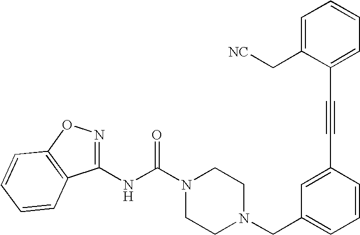

MS: 476.2. ¹H NMR (d₆-DMSO): 9.89 (s, 1H), 7.82-7.79 (m, 1H), 7.65-7.61 (m, 2H), 7.61-7.58 (m, 2H), 7.56-7.53 (m, 2H), 7.50-7.46 (m, 1H), 7.46-7.41 (m, 3H), 7.32-7.29 (m, 1H), 4.21 (s, 2H), 3.60-3.51 (m, 6H), 2.46-2.42 (m, 4H).

### Example 93

### Methyl {2-[(3-{[4-(1,2-benzisoxazol-3-ylcarbamoyl)piperazin-1-yl]methyl}phenyl)ethynyl]phenyl}acetate

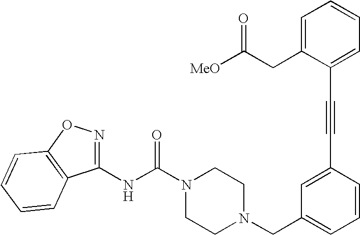

MS: 509.2. ¹H NMR (d₆-DMSO): 9.88 (s, 1H), 7.82-7.79 (m, 1H), 7.65-7.53 (m, 3H), 7.51-7.48 (m, 1H), 7.45-7.27 (m, 7H), 3.93 (s, 2H), 3.63 (s, 3H), 3.58-3.52 (m, 6H), 2.47-2.41 (m, 4H).

### Example 94

### 4-[3-(2-o-Tolyl-ethyl)-benzyl]-piperazine-1-carboxylic acid benzo[d]isoxazol-3-ylamide hydrochloride salt

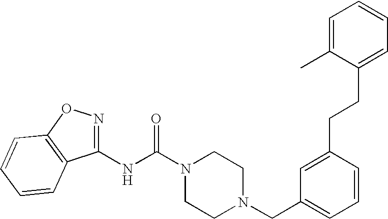

Step A: 4-(3-o-Tolylethynyl-benzyl)-piperazine-1-carboxylic acid tert-butyl ester. The title compound was prepared using methods analogous to those described in Example 63, Step C. MS: 391.3.

Step B: 4-[3-(2-o-Tolyl-ethyl)-benzyl]-piperazine-1-carboxylic acid tert-butyl ester. To a solution of 4-(3-o-tolylethynyl-benzyl)-piperazine-1-carboxylic acid tert-butyl ester (489.4 mg) in EtOH (20 mL) was added 10% Pd/C (139 mg). The flask was purged with N₂, and then fitted with a H₂ balloon. The mixture was stirred at rt for 2 h under 1 atm of H₂, then filtered through diatomaceous earth and concentrated to give 4-[3-(2-o-tolyl-ethyl)-benzyl]-piperazine-1-carboxylic acid tert-butyl ester (480.2 mg, 97%). MS: 395.3.

Step C: 1-[3-(2-o-Tolyl-ethyl)-benzyl]-piperazine. The title compound was prepared using methods analogous to those described in Example 1, Step B. MS: 295.2.

Step D: 4-[3-(2-o-Tolyl-ethyl)-benzyl]-piperazine-1-carboxylic acid benzo[d]isoxazol-3-ylamide hydrochloride salt. The title compound was prepared using methods analogous to those described in Example 1, Step C.

MS: 455.3. ¹H NMR (d₆-DMSO): 7.87-7.83 (m, 1H), 7.68-7.60 (m, 2H), 7.46-7.30 (m, 5H), 7.16-7.07 (m, 4H), 4.37-4.25 (m, 4H), 3.47-3.25 (br hump, 2H), 3.13-3.02 (m, 2H), 2.90-2.85 (m, 4H), 2.27 (s, 3H).

### Example 95

### 4-[3-(Pyrimidin-2-yloxy)-benzyl]-piperazine-1-carboxylic acid benzo[d]isoxazol-3-ylamide

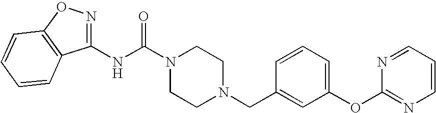

Step A: 4-[3-(pyrimidin-2-yloxy)-benzyl]-piperazine-1-carboxylic acid tert-butyl ester. To a solution of 4-(3-hydroxy-benzyl)-piperazine-1-carboxylic acid tert-butyl ester (500.2 mg) in DMSO (5 mL) were added cesium carbonate (1.10 g) and 2-chloropyrimidine (236.2 mg). The reaction mixture was heated at 60° C. for 18 h, then was cooled to rt, poured into H₂O, and extracted with EtOAc (3×). The organic layers were combined, dried (Na₂SO₄), and concentrated. The crude residue was purified (FCC) to give 4-[3-(pyrimidin-2-yloxy)-benzyl]-piperazine-1-carboxylic acid tert-butyl ester (452.8 mg, 71%).

MS: 371.5.

Step B: 2-(3-Piperazin-1-ylmethyl-phenoxy)-pyrimidine. The title compound was prepared using methods analogous to those described in Example 1, Step B. MS: 271.2.

Step C: 4-[3-(Pyrimidin-2-yloxy)-benzyl]-piperazine-1-carboxylic acid benzo[d]isoxazol-3-ylamide. The title compound was prepared using methods analogous to those described in Example 1, Step C. MS: 431.5. ¹H NMR (d₄-MeOH): 8.61 (d, J=4.8, 2H), 7.87-7.82 (m, 1H), 7.62-7.57 (m, 1H), 7.55-7.53 (m, 1H), 7.43 (t, J=7.8, 1H), 7.34-7.28 (m, 2H), 7.25-7.22 (m, 2H), 7.14-7.10 (m, 1H), 3.69-3.63 (m, 6H), 2.64-2.54 (m, 4H).

The compounds in Examples 96-97 were prepared using methods analogous to those described in Example 95.

### Example 96

### N-1,2-Benzisoxazol-3-yl-4-[3-(pyridin-2-yloxy)benzyl]piperazine-1-carboxamide

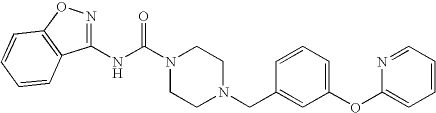

MS: 430.5. ¹H NMR (d₄-MeOH): 8.19-8.14 (m, 1H), 7.88-7.83 (m, 2H), 7.62-7.57 (m, 1H), 7.56-7.53 (m, 1H), 7.43-7.39 (m, 1H), 7.34-7.30 (m, 1H), 7.27-7.24 (m, 1H), 7.19-7.13 (m, 2H), 7.07-7.03 (m, 1H), 6.99-6.95 (m, 1H), 3.69-3.61 (m, 6H), 2.63-2.54 (m, 4H).

### Example 97

### N-1,2-Benzisoxazol-3-yl-4-[3-(pyrazin-2-yloxy)benzyl]piperazine-1-carboxamide

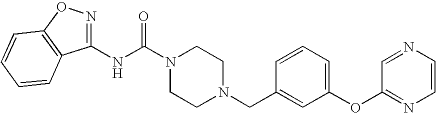

MS: 431.5. ¹H NMR (d₄-MeOH): 8.42 (d, J=1.3, 1H), 8.29 (d, J=2.7, 1H), 8.15-8.14 (m, 1H), 7.85-7.82 (m, 1H), 7.59-7.55 (m, 1H), 7.53-7.51 (m, 1H), 7.42 (t, J=7.9, 1H), 7.32-7.26 (m, 2H), 7.23-7.21 (m, 1H), 7.11-7.08 (m, 1H), 3.67-3.60 (m, 6H), 2.60-2.54 (m, 4H).

### Example 98

### 4-[3-(2-Cyano-benzyloxy)-benzyl]-piperazine-1-carboxylic acid benzo[d]isoxazol-3-ylamide

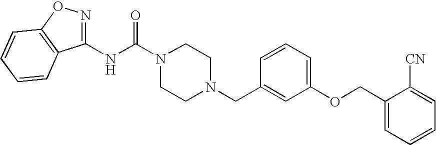

To a solution of 4-(3-hydroxy-benzyl)-piperazine-1-carboxylic acid benzo[d]isoxazol-3-ylamide (100.2 mg) in acetonitrile (1 mL) were added potassium carbonate (75.6 mg) and α-bromo-o-tolunitrile (62.9 mg). The reaction mixture was heated at 50° C. for 18 h, then was cooled to rt, poured into H₂O, and extracted with EtOAc (3×). The organic layers were combined, dried (Na₂SO₄), and concentrated. The crude residue was purified (FCC) to give the title compound (41.3 mg, 31%). MS: 468.2. ¹H NMR (d₄-MeOH): 7.84-7.81 (m, 1H), 7.80-7.78 (m, 1H), 7.72-7.68 (m, 2H), 7.60-7.56 (m, 1H), 7.54-7.50 (m, 2H), 7.32-7.26 (m, 2H), 7.09-7.07 (m, 1H), 7.00-6.95 (m, 2H), 5.29-5.26 (m, 2H), 3.64-3.60 (m, 4H), 3.59-3.57 (m, 2H), 2.55-2.50 (m, 4H).

### Example 99

### N-1,2-Benzisoxazol-3-yl-4-[3-(benzyloxy)benzyl]piperazine-1-carboxamide

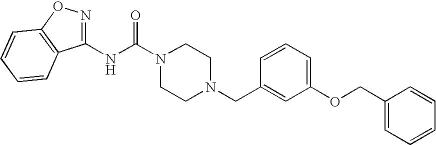

The title compound was prepared using methods analogous to those described in Example 98. MS: 443.2. ¹H NMR (d₄-MeOH): 7.84-7.81 (m, 1H), 7.59-7.56 (m, 1H), 7.54-7.51 (m, 1H), 7.45-7.42 (m, 2H), 7.38-7.34 (m, 2H), 7.32-7.28 (m, 2H), 7.26-7.23 (m, 1H), 7.03-7.01 (m, 1H), 6.95-6.90 (m, 2H), 5.10 (s, 2H), 3.63-3.59 (m, 4H), 3.56 (s, 2H), 2.54-2.47 (m, 4H).

The compounds in Examples 100-203 were prepared using methods analogous to those described in Example 1.

### Example 100

### 4-(1H-Benzimidazol-6-ylmethyl)-N-1,2-benzisoxazol-3-ylpiperazine-1-carboxamide.

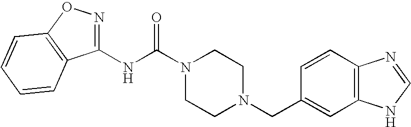

MS: 377.5. ¹H NMR (d₆-DMSO): 8.18 (s, 1H), 7.81-7.78 (td, J=8.0, 1.0, 1H), 7.61-7.50 (m, 4H), 7.28-7.24 (m, 1H), 7.19-7.16 (dd, J=8.2, 1.4, 1H), 3.62 (s, 2H), 3.55-3.50 (m, 4H), 2.44-2.40 (m, 4H).

### Example 101

### N-1,2-Benzisoxazol-3-yl-4-(1H-indazol-6-ylmethyl)piperazine-1-carboxamide

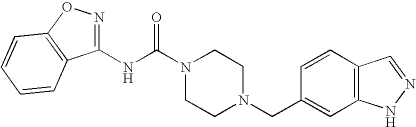

MS: 377.4. ¹H NMR (d₆-DMSO): 13.00 (s, 1H), 9.85 (s, 1H), 8.03 (d, J=0.8, 1H), 7.79 (d, J=8.0, 1H), 7.66 (s, 1H), 7.61-7.55 (m, 2H), 7.50 (d, J=8.5, 1H), 7.37-7.33 (dd, J=8.6, 1.4, 1H), 7.27 (t, J=7.9, 1H), 3.61 (s, 2H), 3.55-3.50 (m, 4H), 2.45-2.40 (m, 4H).

### Example 102

### N-1,2-Benzisoxazol-3-yl-4-[4-(methylsulfonyl)benzyl]piperazine-1-carboxamide

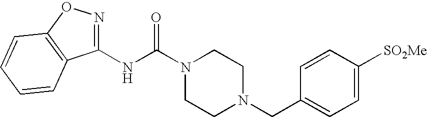

MS: 415.4. ¹H NMR (d₆-DMSO): 9.88 (s, 1H), 7.91 (d, J=8.4, 1H), 7.81 (d, J=8.0, 1H), 7.64-7.56 (m, 4H), 7.31-7.27 (m, 1H), 3.65 (s, 2H), 3.57-3.53 (m, 4H), 3.22 (s, 3H), 2.46-2.41 (m, 4H).

### Example 103

### N-1,2-Benzisoxazol-3-yl-4-[4-(trifluoromethoxy)benzyl]piperazine-1-carboxamide

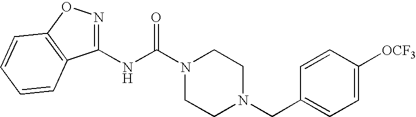

MS: 421.4. ¹H NMR (d₆-DMSO): 9.83 (s, 1H), 7.82-7.79 (td, J=8.1, 0.9, 2H), 7.63-7.56 (m, 2H), 7.47 (d, J=8.7, 1H), 7.35-7.27 (m, 3H), 3.56 (s, 2H), 3.55-3.51 (m, 4H), 2.44-2.40 (m, 4H).

### Example 104

### 4-[3-(4-Chlorophenoxy)benzyl]-N-(6-methoxypyridazin-3-yl)piperazine-1-carboxamide

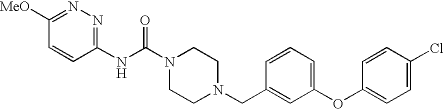

MS: 454.2. ¹H NMR (d₆-DMSO): 8.10 (d, J=9.6, 1H), 7.41 (d, J=8.9, 2H), 7.38 (t, J=7.8, 1H), 7.19 (d, J=7.5, 1H), 7.11-7.09 (m, 1H), 7.06-7.03 (m, 3H), 6.96-6.93 (dd, J=7.9, 2.3, 1H), 3.99 (s, 3H), 3.63-3.59 (m, 4H), 3.58 (s, 2H), 2.52-2.47 (m, 4H).

### Example 105

### N-1,2-Benzisoxazol-3-yl-4-[4-chloro-3-(trifluoromethoxy)benzyl]-piperazine-1-carboxamide

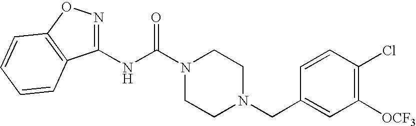

MS: 455.4. ¹H NMR (d₆-DMSO): 9.87 (s, 1H), 7.80 (d, J=8.0, 1H), 7.67 (d, J=8.2, 1H), 7.64-7.57 (m, 2H), 7.52 (s, 1H), 7.44-7.41 (dd, J=8.3, 1.8, 1H), 7.31-7.27 (m, 1H), 3.60 (s, 2H), 3.56-3.52 (m, 4H), 2.46-2.40 (m, 4H),

### Example 106

### N-1,2-Benzisoxazol-3-yl-4-[4-fluoro-3-(trifluoromethoxy)benzyl]-piperazine-1-carboxamide

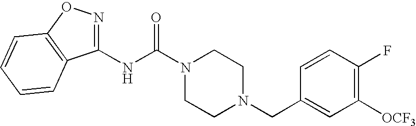

MS: 439.4. ¹H NMR (d₆-DMSO): 9.83 (s, 1H), 7.82-7.79 (td, J=8.1, 1.0, 1H), 7.64-7.56 (m, 2H), 7.52-7.40 (m, 3H), 7.32-7.27 (m, 1H), 3.57 (s, 2H), 3.56-3.51 (m, 4H), 2.46-2.40 (m, 4H).

### Example 107

### N-1,2-Benzisoxazol-3-yl-4-[3-chloro-4-(trifluoromethoxy)benzyl]-piperazine-1-carboxamide

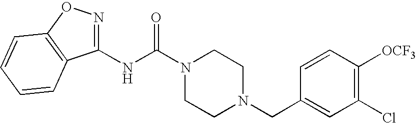

MS: 455.4. ¹H NMR (d₆-DMSO): 9.88 (s, 1H), 7.81 (d, J=8.0, 1H), 7.67-7.52 (m, 4H), 7.47-7.43 (dd, J=8.4, 2.0, 1H), 7.32-7.28 (m, 1H), 3.58 (s, 2H), 3.57-3.51 (m, 4H), 2.46-2.41 (m, 4H).

### Example 108

### N-1,2-Benzisoxazol-3-yl-4-[3-fluoro-4-(trifluoromethoxy)benzyl]-piperazine-1-carboxamide

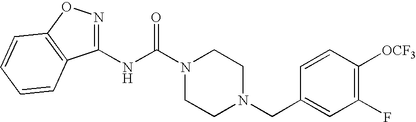

MS: 439.4. ¹H NMR (d₆-DMSO): 9.88 (s, 1H), 7.81 (d, J=8.0, 1H), 7.64-7.58 (m, 2H), 7.54 (t, J=8.2, 1H), 7.49-7.45 (dd, J=11.4, 1.8, 1H), 7.33-7.28 (m, 2H), 3.58 (s, 2H), 3.57-3.51 (m, 4H), 2.45-2.42 (m, 4H).

### Example 109

### N-1,2-Benzisoxazol-3-yl-4-{3-[4-(trifluoromethyl)phenoxy]benzyl}-piperazine-1-carboxamide

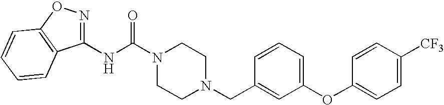

MS: 497.5. ¹H NMR (CDCl₃): 8.08 (d, J=8.1, 1H), 7.87 (s, 1H), 7.60 (d, J=8.9, 2H), 7.56-7.52 (m, 1H), 7.47 (d, J=8.5, 1H), 7.37 (t, J=7.9, 1H), 7.32-7.28 (m, 1H), 7.19 (d, J=7.4, 1H), 7.12-7.10 (m, 1H), 7.07 (d, J=8.4, 2H), 7.00-6.97 (m, 1H), 3.69-3.62 (m, 4H), 3.59 (s, 2H), 2.60-2.53 (m, 4H).

### Example 110

### N-1,2-Benzisoxazol-3-yl-4-(3-phenoxybenzyl)piperazine-1-carboxamide

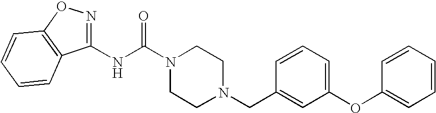

MS: 429.5. ¹H NMR (CDCl₃): 8.09 (d, J=8.1, 1H), 7.80 (s, 1H), 7.57-7.52 (m, 1H), 7.48 (d, J=8.5, 1H), 7.39-7.29 (m, 4H), 7.16-7.01 (m, 5H), 6.96-6.92 (m, 1H), 3.67-3.60 (m, 4H), 3.57 (s, 2H), 2.60-2.51 (m, 4H).

### Example 111

### N-1,2-Benzisoxazol-3-yl-4-(3,4-dichlorobenzyl)piperazine-1-carboxamide

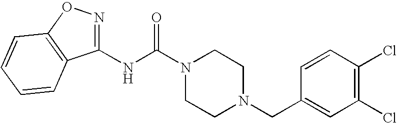

MS: 405.4. ¹H NMR (CDCl₃): 8.08 (d, J=8.0, 1H), 7.94 (s, 1H), 7.57-7.53 (m, 1H), 7.50-7.46 (m, 2H), 7.43 (d, J=8.2, 1H), 7.32-7.28 (m, 1H), 7.22-7.19 (dd, J=8.2, 2.0, 1H), 3.68-3.63 (m, 4H), 3.53 (s, 2H), 2.59-2.52 (m, 4H).

### Example 112

### N-1,2-Benzisoxazol-3-yl-4-[4-(benzyloxy)benzyl]piperazine-1-carboxamide

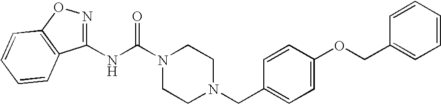

MS: 443.5. ¹H NMR (CDCl₃): 8.09 (d, J=8.1, 1H), 7.80 (s, 1H), 7.57-7.52 (m, 1H), 7.50-7.25 (m, 9H), 6.97 (d, J=8.6, 2H), 5.09 (s, 2H), 3.66-3.61 (m, 4H), 3.52 (s, 2H), 2.58-2.50 (m, 4H).

### Example 113

### N-1,2-Benzisoxazol-3-yl-4-(1-benzothiophen-2-ylmethyl)piperazine-1-carboxamide

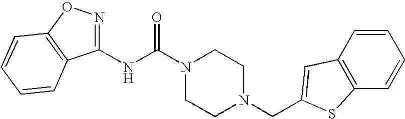

MS: 393.4. ¹H NMR (CDCl₃): 8.08 (d, J=8.1, 1H), 7.83 (d, J=8.1, 1H), 7.77 (s, 1H), 7.73 (d, J=8.0, 1H), 7.56-7.52 (m, 1H), 7.47 (d, J=8.5, 1H), 7.38-7.29 (m, 3H), 7.20 (s, 1H), 3.88 (s, 2H), 3.71-3.63 (m, 4H), 2.69-2.62 (m, 4H).

### Example 114

### N-1,2-Benzisoxazol-3-yl-4-[3-(quinolin-6-yloxy)benzyl]piperazine-1-carboxamide

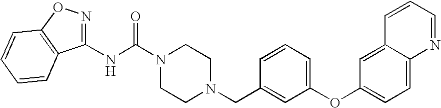

MS: 480.5. ¹H NMR (CDCl₃): 8.88-8.85 (dd, J=4.2, 1.7, 1H), 8.12 (d, J=9.2, 1H), 8.08 (d, J=8.1, 1H), 8.04 (d, J=8.2, 1H), 7.64 (s, 1H), 7.56-7.50 (m, 2H), 7.48 (d, J=8.5, 1H), 7.42-7.35 (m, 2H), 7.31-7.26 (m, 2H), 7.18 (d, J=7.7, 1H), 7.16-7.13 (m, 1H), 7.05-7.01 (m, 1H), 3.66-3.58 (m, 6H), 2.62-2.53 (m, 4H).

### Example 115

### N-1,2-Benzisoxazol-3-yl-4-(4-bromo-3-fluorobenzyl)piperazine-1-carboxamide

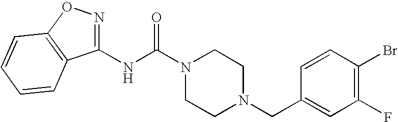

MS: 433.4. ¹H NMR (CDCl₃): 8.08 (d, J=8.0, 1H), 7.91 (s, 1H), 7.58-7.46 (m, 3H), 7.32-7.29 (m, 1H), 7.21-7.17 (dd, J=9.4, 1.8, 1H), 7.05-7.02 (dd, J=8.1, 1.4, 1H), 3.69-3.61 (m, 4H), 3.54 (s, 2H), 2.59-2.51 (m, 4H).

### Example 116

### N-1,2-Benzisoxazol-3-yl-4-(1,3-benzodioxol-5-ylmethyl)piperazine-1-carboxamide

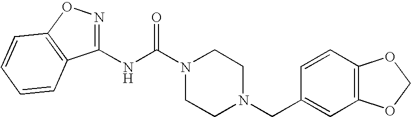

MS: 381.4. ¹H NMR (CDCl₃): 8.13-8.06 (m, 2H), 7.57-7.52 (m, 1H), 7.47 (d, J=8.5, 1H), 7.32-7.28 (m, 1H), 6.90 (s, 1H), 6.80-6.76 (m, 2H), 5.98 (s, 2H), 3.69-3.62 (m, 4H), 3.49 (s, 2H), 2.57-2.50 (m, 4H).

### Example 117

### N-1,2-Benzisoxazol-3-yl-4-(quinolin-3-ylmethyl)piperazine-1-carboxamide

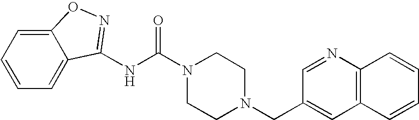

MS: 388.5. ¹H NMR (CDCl₃): 8.94 (d, J=2.1, 1H), 8.62 (s, 1H), 8.15-8.04 (m, 3H), 7.82 (d, J=9.2, 1H), 7.74-7.69 (m, 1H), 7.59-7.48 (m, 2H), 7.41 (d, J=8.5, 1H), 7.29-7.24 (m, 1H), 3.75 (s, 2H), 3.72-3.67 (m, 4H), 2.65-2.56 (m, 4H).

### Example 118

### N-1,2-Benzisoxazol-3-yl-4-(1H-indol-5-ylmethyl)piperazine-1-carboxamide

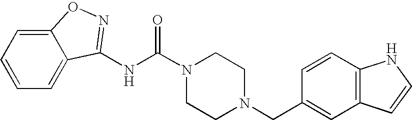

MS: 376.5. ¹H NMR (CDCl₃): 8.18 (s, 1H), 8.08 (d, J=8.0, 1H), 7.99 (s, 1H), 7.58 (s, 1H), 7.54-7.49 (m, 1H), 7.44 (d, J=8.5, 1H), 7.37 (d, J=8.3, 1H), 7.29-7.24 (m, 1H), 7.23-7.17 (m, 2H), 6.56-6.51 (m, 1H), 3.67 (s, 2H), 3.65-3.60 (m, 4H), 2.61-2.53 (m, 4H).

### Example 119

### N-1,2-Benzisoxazol-3-yl-4-[3-(naphthalen-2-yloxy)benzyl]piperazine-1-carboxamide

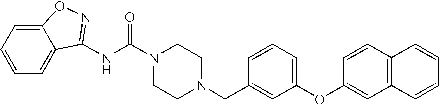

MS: 479.5. ¹H NMR (CDCl₃): 8.11-8.04 (m, 2H), 7.85-7.80 (m, 2H), 7.70 (d, J=8.1, 1H), 7.53-7.38 (m, 4H), 7.35-7.24 (m, 4H), 7.11 (d, J=7.5, 1H), 7.00-6.96 (m, 1H), 3.66-3.60 (m, 4H), 3.56 (s, 2H), 2.58-2.51 (m, 4H).

### Example 120

### N-1,2-Benzisoxazol-3-yl-4-(4-bromobenzyl)piperazine-1-carboxamide

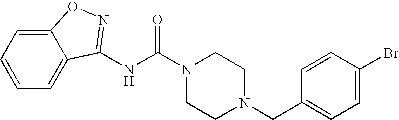

MS: 415.4. ¹H NMR (CDCl₃): 8.68 (s, 1H), 8.07 (d, J=8.0, 1H), 7.55-7.40 (m, 4H), 7.30-7.20 (m, 3H), 3.70-3.62 (m, 4H), 3.50 (s, 2H), 2.56-2.47 (m, 4H).

### Example 121

### N-1,2-Benzisoxazol-3-yl-4-(3,4-dibromobenzyl)piperazine-1-carboxamide

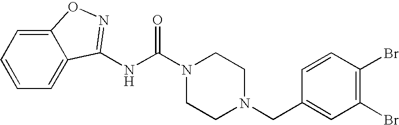

MS: 493.3. ¹H NMR (CDCl₃): 8.73 (s, 1H), 8.07 (d, J=8.1, 1H), 7.63 (d, J=2.0, 1H), 7.57 (d, J=8.2, 1H), 7.55-7.50 (m, 1H), 7.43 (d, J=8.5, 1H), 7.30-7.25 (m, 1H), 7.17-7.13 (m, 1H), 3.71-3.64 (m, 4H), 3.48 (s, 2H), 2.56-2.48 (m, 4H).

### Example 122

### N-1,2-Benzisoxazol-3-yl-4-[3-(2-chlorophenoxy)benzyl]piperazine-1-carboxamide

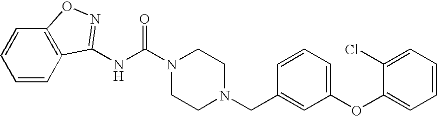

MS: 463.5. ¹H NMR (d₆-DMSO): 9.86 (s, 1H), 7.80 (d, J=8.0, 1H), 7.65-7.57 (m, 3H), 7.40-7.28 (m, 3H), 7.26-7.21 (m, 1H), 7.13-7.09 (m, 2H), 6.94 (s, 1H), 6.86-6.83 (m, 1H), 3.55-3.47 (m, 6H), 2.44-2.37 (m, 4H).

### Example 123

### 4-Naphthalen-2-ylmethyl-piperazine-1-carboxylic acid benzo[d]isoxazol-3-ylamide

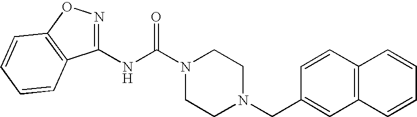

MS: 387.5. ¹H NMR (CDCl₃): 8.07 (d, J=8.4, 1H), 7.85-7.82 (m, 3H), 7.76 (s, 1H), 7.53-7.45 (m, 6H), 7.28-7.26 (m, 1H), 3.73 (s, 2H), 3.63 (t, J=4.8, 4H), 2.59 (t, J=4.8, 4H).

### Example 124

### 4-Quinolin-2-ylmethyl-piperazine-1-carboxylic acid benzo[d]isoxazol-3-ylamide

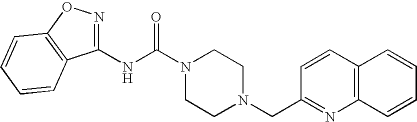

MS: 388.5. ¹H NMR (CDCl₃): 8.17 (d, J=8.4, 1H), 8.10-8.07 (m, 2H), 7.83 (d, J=7.8, 1H), 7.74-7.71 (m, 1H), 7.64 (d, J=8.4, 1H), 7.56-7.51 (m, 3H), 7.46 (d, J=7.8, 1H), 7.29-7.26 (m, 1H), 3.92 (s, 2H), 3.66 (t, J=4.8, 4H), 2.67 (t, J=4.8, 4H).

### Example 125

### 4-[3-(4-Cyano-phenoxy)-benzyl]-piperazine-1-carboxylic acid benzo[d]isoxazol-3-ylamide

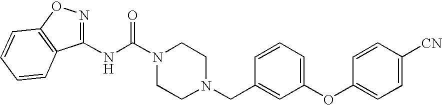

MS: 454.5. ¹H NMR (CDCl₃): 8.07 (d, J=7.8, 1H), 7.91 (s, 1H), 7.62-7.60 (m, 2H), 7.54-7.51 (m, 1H), 7.45 (d, J=8.4, 1H), 7.38 (t, J=7.8, 1H), 7.29-7.26 (m, 1H), 7.21 (d, J=7.2, 1H), 7.10-7.09 (m, 1H), 7.03-7.01 (m, 2H), 6.99-6.97 (dd, J=1.8, 7.2, 1H), 3.64 (t, J=4.8, 4H), 3.58 (s, 2H), 2.55 (t, J=4.8, 4H).

### Example 126

### 4-Benzofuran-2-ylmethyl-piperazine-1-carboxylic acid benzo[d]isoxazol-3-ylamide

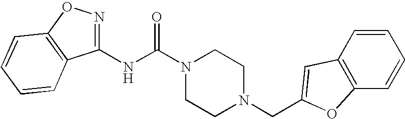

MS: 377.4. ¹H NMR (CDCl₃): 8.07 (d, J=7.8, 1H), 8.05 (s, 1H), 7.56-7.50 (m, 3H), 7.45 (d, J=8.4, 1H), 7.30-7.27 (m, 2H), 7.25-7.22 (m, 1H), 6.65 (s, 1H), 3.78 (s, 2H), 3.70 (t, J=4.8, 4H), 2.67 (t, J=4.8, 4H).

### Example 127

### N-1,2-Benzisoxazol-3-yl-4-[3-(3-chlorophenoxy)benzyl]piperazine-1-carboxamide

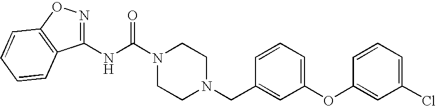

MS: 463.5. ¹H NMR (d₄-MeOH): 7.85-7.82 (m, 1H), 7.59-7.55 (m, 1H), 7.53-7.51 (m, 1H), 7.39-7.35 (m, 1H), 7.34-7.28 (m, 2H), 7.19-7.17 (m, 1H), 7.12-7.07 (m, 2H), 6.97-6.94 (m, 2H), 6.93-6.91 (m, 1H), 3.65-3.58 (m, 6H), 2.57-2.51 (m, 4H).

### Example 128

### N-1,2-Benzisoxazol-3-yl-4-{3-[4-cyano-3-(trifluoromethyl)phenoxy]benzyl}piperazine-1-carboxamide

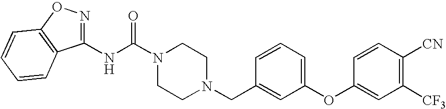

MS: 522.2. ¹H NMR (d₄-MeOH): 7.94 (d, J=8.6, 1H), 7.84-7.82 (m, 1H), 7.59-7.55 (m, 1H), 7.53-7.51 (m, 1H), 7.48 (t, J=7.9, 1H), 7.42 (d, J=2.4, 1H), 7.35-7.26 (m, 3H), 7.22-7.20 (m, 1H), 7.10-7.07 (m, 1H), 3.65-3.61 (m, 6H), 2.58-2.53 (m, 4H).

### Example 129

### N-1,2-Benzisoxazol-3-yl-4-[3-(3-cyanophenoxy)benzyl]piperazine-1-carboxamide

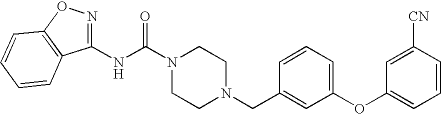

¹H NMR (d₆-DMSO): 9.94-9.75 (m, 1H), 7.89-7.68 (m, 1H), 7.67-7.54 (m, 4H), 7.53-7.49 (m, 1H), 7.43-7.38 (m, 1H), 7.37-7.33 (m, 1H), 7.32-7.28 (m, 1H), 7.21-7.17 (m, 1H), 7.08-7.05 (m, 1H), 7.00-6.96 (m, 1H), 3.59-3.46 (m, 6H), 2.46-2.39 (m, 4H).

### Example 130

### N-1,2-Benzisoxazol-3-yl-4-(3-{4-[(trifluoromethyl)sulfanyl]phenoxy}-benzyl)piperazine-1-carboxamide

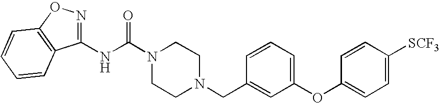

MS: 529.2. ¹H NMR (d₄-MeOH): 7.89-7.81 (m, 1H), 7.70-7.64 (m, 2H), 7.62-7.56 (m, 1H), 7.55-7.52 (m, 1H), 7.44-7.38 (m, 1H), 7.35-7.28 (m, 1H), 7.26-7.22 (m, 1H), 7.17-7.13 (m, 1H), 7.09-7.05 (m, 2H), 7.04-7.01 (m, 1H), 3.69-3.58 (m, 6H), 2.64-2.49 (m, 4H).

### Example 131

### N-1,2-Benzisoxazol-3-yl-4-{3-[(2,2-difluoro-1,3-benzodioxol-5-yl)oxy]benzyl}piperazine-1-carboxamide

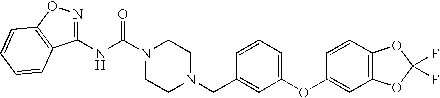

MS: 509.2. ¹H NMR (d₄-MeOH): 7.87-7.83 (m, 1H), 7.62-7.56 (m, 1H), 7.55-7.52 (m, 1H), 7.39-7.29 (m, 2H), 7.21-7.14 (m, 2H), 7.08-7.05 (m, 1H), 6.97-6.92 (m, 2H), 6.84-6.77 (m, 1H), 3.69-3.61 (m, 4H), 3.61-3.59 (m, 2H), 2.63-2.47 (m, 4H).

### Example 132

### N-1,2-Benzisoxazol-3-yl-4-(3-{4-[(trifluoromethyl)sulfonyl]phenoxy}-benzyl)piperazine-1-carboxamide

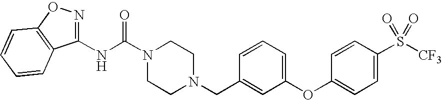

MS: 561.2. ¹H NMR (d₄-MeOH): 8.10-8.02 (m, 2H), 7.87-7.81 (m, 1H), 7.62-7.56 (m, 1H), 7.56-7.47 (m, 2H), 7.38-7.28 (m, 2H), 7.29-7.23 (m, 3H), 7.15-7.09 (m, 1H), 3.80-3.45 (m, 6H), 2.67-2.47 (m, 4H).

### Example 133

### N-1,2-Benzisoxazol-3-yl-4-{[3-(phenylethynyl)phenyl]methyl}piperazine-1-carboxamide

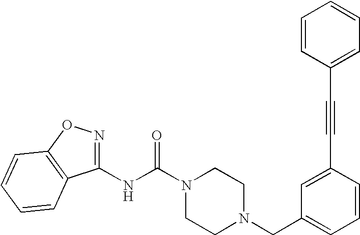

MS: 437.2. ¹H NMR (CDCl₃): 8.14-7.98 (m, 1H), 7.62-7.33 (m, 12H), 3.75-3.48 (m, 6H), 2.68-2.48 (m, 4H).

### Example 134

### N-Isoxazol-3-yl-4-{3-[4-(trifluoromethoxy)phenoxy]benzyl}piperazine-1-carboxamide

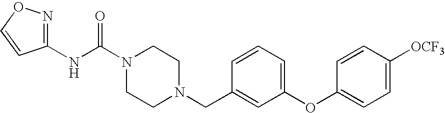

MS: 463.2. ¹H NMR (d₆-DMSO): 9.99-9.91 (m, 1H), 8.66-8.63 (m, 1H), 7.50-7.44 (m, 1H), 7.39-7.34 (m, 2H), 7.27-7.15 (m, 2H), 7.14-7.09 (m, 3H), 6.72-6.70 (m, 1H), 4.36-4.26 (m, 2H), 4.22-4.13 (m, 2H), 3.35-3.24 (m, 2H), 3.16-2.94 (m, 4H).

### Example 135

### 4-[4-(Benzyloxy)benzyl]-N-isoxazol-3-ylpiperazine-1-carboxamide

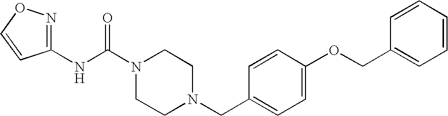

MS: 393.5. ¹H NMR (CDCl₃): 8.22 (d, J=1.6, 1H), 7.85 (s, 1H), 7.48-7.32 (m, 5H), 7.25 (d, J=8.6, 2H), 6.99 (d, J=1.8, 1H), 6.96 (d, J=8.7, 2H), 5.08 (s, 2H), 3.58-3.52 (m, 4H), 3.50 (s, 2H), 2.54-2.44 (m, 4H).

### Example 136

### 4-[3-(3-Chlorophenoxy)benzyl]-N-isoxazol-3-ylpiperazine-1-carboxamide

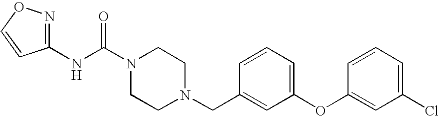

MS: 413.4. ¹H NMR (CDCl₃): 8.23 (d, J=1.7, 1H), 7.81 (s, 1H), 7.34 (t, J=7.8, 1H), 7.29-7.25 (m, 1H), 7.15-7.04 (m, 3H), 7.01-6.98 (m, 2H), 6.96-6.90 (m, 2H), 3.58-3.53 (m, 6H), 2.55-2.47 (m, 4H).

### Example 137

### N-Isoxazol-3-yl-4-{3-[4-(2,2,2-trifluoroethoxy)phenoxy]benzyl}piperazine-1-carboxamide

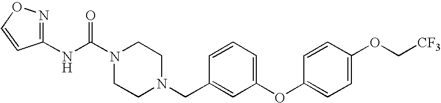

MS: 477.5. ¹H NMR (CDCl₃): 8.22 (d, J=1.7, 1H), 8.07 (s, 1H), 7.31-7.25 (m, 1H), 7.06 (d, J=7.8, 1H), 7.03-6.98 (m, 4H), 6.95 (d, J=9.2, 2H), 6.88-6.85 (dd, J=8.1, 1.8, 1H), 4.39-4.33 (q, J=8.1, 2H), 3.60-3.55 (m, 4H), 3.53 (s, 2H), 2.55-2.43 (m, 4H).

### Example 138

### 4-(1-Benzofuran-2-ylmethyl)-N-isoxazol-3-ylpiperazine-1-carboxamide

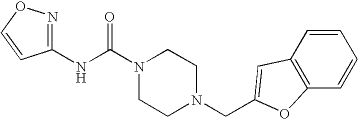

MS: 327.4. ¹H NMR (CDCl₃): 8.23 (s, 1H), 8.21 (d, J=1.8, 1H), 7.56 (d, J=7.5, 1H), 7.51 (d, J=8.1, 1H), 7.31-7.27 (m, 1H), 7.26-7.22 (dt, J=7.5, 1.0, 1H), 6.99 (d, J=1.7, 1H), 6.64 (s, 1H), 3.76 (s, 2H), 3.66-3.60 (m, 4H), 2.66-2.59 (m, 4H).

### Example 139

### 4-[3-(3-Cyanophenoxy)benzyl]-N-isoxazol-3-ylpiperazine-1-carboxamide

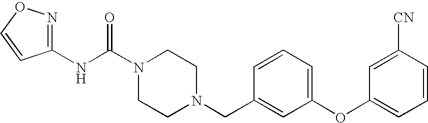

MS: 404.5. ¹H NMR (d₆-DMSO): 9.72 (s, 1H), 8.66 (d, J=1.7, 1H), 7.62-7.57 (m, 2H), 7.51-7.49 (m, 1H), 7.42-7.38 (m, 1H), 7.36-7.33 (m, 1H), 7.17 (d, J=7.6, 1H), 7.04 (s, 1H), 6.99-6.96 (m, 1H), 6.76 (d, J=1.7, 1H), 3.52 (s, 2H), 3.46-3.43 (m, 4H), 2.39-2.34 (m, 4H).

### Example 140

### 4-[3-(2-Chlorophenoxy)benzyl]-N-isoxazol-3-ylpiperazine-1-carboxamide

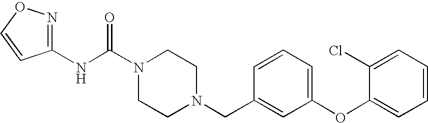

MS: 413.4. ¹H NMR (d₆-DMSO): 9.72 (s, 1H), 8.66 (d, J=1.7, 1H), 7.63-7.58 (m, 1H), 7.40-7.32 (m, 2H), 7.25-7.21 (m, 1H), 7.13-7.07 (m, 2H), 6.92 (s, 1H), 6.85-6.82 (m, 1H), 6.76 (d, J=1.7, 1H), 3.49 (s, 2H), 3.46-3.41 (m, 4H), 2.37-2.33 (m, 4H).

### Example 141

### 4-{3-[(2,2-Difluoro-1,3-benzodioxol-5-yl)oxy]benzyl}-N-isoxazol-3-ylpiperazine-1-carboxamide

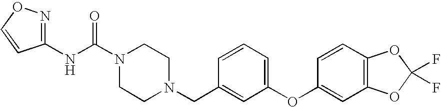

MS: 459.5. ¹H NMR (d₆-DMSO): 9.72 (s, 1H), 8.66 (d, J=1.6, 1H), 7.42 (d, J=8.8, 1H), 7.37-7.32 (m, 1H), 7.27 (d, J=2.4, 1H), 7.10 (d, J=7.6, 1H), 6.98 (s, 1H), 6.91-6.88 (m, 1H), 6.87-6.83 (m, 1H), 6.76 (d, J=1.7, 1H), 3.49 (s, 2H), 3.47-3.42 (m, 4H), 2.39-2.32 (m, 4H).

### Example 142

### 4-(1-Benzothiophen-2-ylmethyl)-N-isoxazol-3-ylpiperazine-1-carboxamide

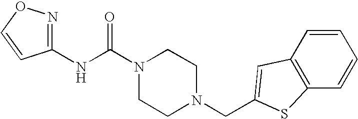

MS: 343.4. ¹H NMR (d₆-DMSO): 9.75 (s, 1H), 8.66 (d, J=1.8, 1H), 7.90 (d, J=7.7, 1H), 7.76 (d, J=7.2, 1H), 7.36-7.28 (m, 3H), 6.77 (d, J=1.8, 1H), 3.82 (s, 2H), 3.53-3.44 (m, 4H), 2.48-2.44 (m, 4H).

### Example 143

### 4-(1,3-Benzodioxol-5-ylmethyl)-N-isoxazol-3-ylpiperazine-1-carboxamide

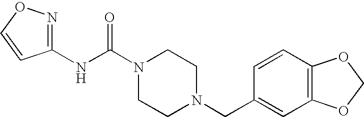

MS: 331.4. ¹H NMR (d₆-DMSO): 9.73 (s, 1H), 8.66 (d, J=1.7, 1H), 6.88-6.83 (m, 2H), 6.77-6.74 (m, 2H), 5.99 (s, 2H), 3.49-3.41 (m, 4H), 3.40 (s, 2H), 2.35-2.31 (m, 4H).

### Example 144

### N-Isoxazol-3-yl-4-(naphthalen-2-ylmethyl)piperazine-1-carboxamide

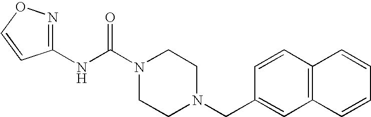

MS: 337.4. ¹H NMR (d₆-DMSO): 9.73 (s, 1H), 8.66 (d, J=1.7, 1H), 7.91-7.87 (m, 3H), 7.81 (s, 1H), 7.54-7.46 (m, 3H), 6.77 (d, J=1.6, 1H), 3.66 (s, 2H), 3.51-3.44 (m, 4H), 2.43-2.39 (m, 4H).

### Example 145

### 4-[3-(4-Bromophenoxy)benzyl]-N-isoxazol-3-ylpiperazine-1-carboxamide

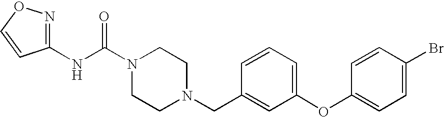

MS: 457.4. ¹H NMR (CDCl₃): 8.22-8.17 (m, 2H), 7.43 (d, J=9.0, 2H), 7.32-7.28 (m, 1H), 7.09 (d, J=7.6, 1H), 7.01 (s, 1H), 6.99 (d, J=1.7, 1H), 6.91-6.87 (m, 3H), 3.58-3.51 (m, 6H), 2.52-2.46 (m, 4H).

### Example 146

### 4-Quinolin-2-ylmethyl-piperazine-1-carboxylic acid isoxazol-3-ylamide

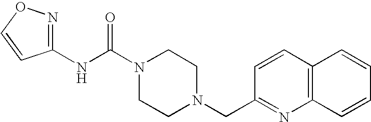

MS: 338.4. ¹H NMR (CDCl₃): 8.19 (d, J=1.2, 1H), 8.15 (d, J=8.4, 1H), 8.08 (d, J=8.4, 1H), 7.92 (br hump, 1H), 7.82-7.81 (m, 1H), 7.73-7.70 (m, 1H), 7.63 (d, J=8.4, 1H), 7.55-7.52 (m, 1H), 6.97 (d, J=1.2, 1H), 3.89 (s, 2H), 3.59 (t, J=4.8, 4H), 2.62 (t, J=4.8, 4H).

### Example 147

### 4-Quinolin-3-ylmethyl-piperazine-1-carboxylic acid isoxazol-3-ylamide

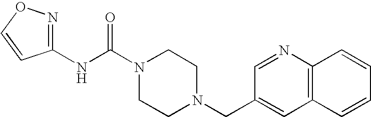

MS: 338.4. ¹H NMR (CDCl₃): 8.92 (d, J=2.4, 1H), 8.21 (d, J=1.8, 1H), 8.11 (d, J=9.0, 1H), 8.07 (d, J=1.2, 1H), 7.83-7.81 (m, 1H), 7.73-7.70 (m, 1H), 7.58-7.55 (m, 1H), 7.48 (br hump, 1H), 6.96 (d, J=1.8, 1H), 3.74 (s, 2H), 3.55 (t, J=4.8, 4H), 2.56 (t, J=4.8, 4H).

### Example 148

### 4-(4-Bromo-benzyl)-piperazine-1-carboxylic acid isoxazol-3-ylamide

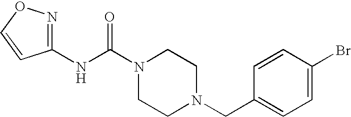

MS: 363.3. ¹H NMR (CDCl₃): 8.19 (d, J=1.2, 1H), 7.46-7.45 (m, 2H), 7.21 (d, J=8.4, 2H), 6.99 (d, J=1.8, 1H), 3.57 (t, J=4.8, 4H), 3.48 (s, 2H), 2.48 (t, J=4.8, 4H).

### Example 149

### 4-(1H-Indol-6-ylmethyl)-piperazine-1-carboxylic acid isoxazol-3-ylamide

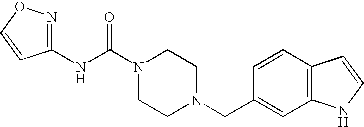

MS: 326.4. ¹H NMR (CDCl₃): 8.19 (d, J=1.8, 1H), 8.16 (br s, 1H), 7.73 (br s, 1H), 7.56 (s, 1H), 7.36 (d, J=8.4, 1H), 7.225-7.216 (m, 1H), 7.19-7.17 (dd, J=1.2, 8.4, 1H), 6.96 (d, J=1.8, 1H), 6.54-6.53 (m, 1H), 3.64 (s, 2H), 3.53 (t, J=4.8, 4H), 2.52 (t, J=4.8, 4H).

### Example 150

### 4-[3-(Naphthalen-2-yloxy)-benzyl]-piperazine-1-carboxylic acid isoxazol-3-ylamide

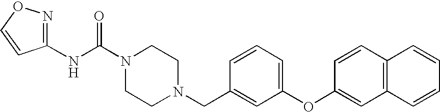

MS: 429.5. ¹H NMR (CDCl₃): 8.19 (d, J=1.2, 1H), 7.88 (br hump, 1H), 7.83 (m, 2H), 7.70 (d, J=9.0, 1H), 7.47-7.46 (m, 1H), 7.45-7.40 (m, 1H), 7.33-7.31 (m, 2H), 7.28-7.24 (m, 1H), 7.11-7.08 (m, 2H), 6.99-6.97 (m, 2H), 3.54-3.49 (m, 6H), 2.50 (br hump, 4H).

### Example 151

### 4-(4-Bromo-3-fluoro-benzyl)-piperazine-1-carboxylic acid isoxazol-3-ylamide

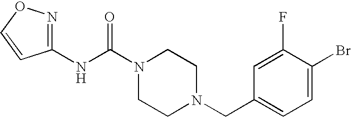

MS: 381.3. ¹H NMR (CDCl₃): 8.20 (d, J=1.8, 1H), 8.13 (br s, 1H), 7.50-7.48 (dd, J=7.2, 7.8, 1H), 7.17-7.15 (dd, J=1.8, 9.0, 1H), 7.01-6.99 (dd, J=1.2, 8.4, 1H), 6.98 (d, J=1.2, 1H), 3.56 (t, J=4.8, 4H), 3.49 (s, 2H), 2.49 (t, J=4.8, 4H).

### Example 152

### 4-[3-(4-Cyano-phenoxy)-benzyl]-piperazine-1-carboxylic acid isoxazol-3-ylamide

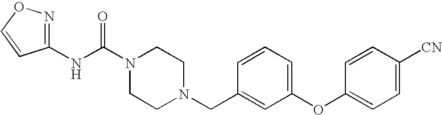

MS: 404.5. ¹H NMR (CDCl₃): 8.20 (d, J=1.2, 1H), 8.07 (br s, 1H), 7.62-7.60 (m, 2H), 7.61 (t, J=7.8, 1H), 7.19 (d, J=7.8, 1H), 7.08 (s, 1H), 7.01 (d, J=9.0, 2H), 6.98-6.96 (m, 2H), 3.56-3.55 (m, 6H), 2.50 (t, J=4.8, 4H).

### Example 153

### 4-[3-(3,4-Difluorophenoxy)benzyl]-N-isoxazol-3-ylpiperazine-1-carboxamide

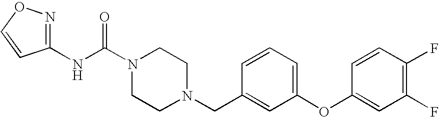

MS: 415.5. ¹H NMR (d₄-MeOH): 8.42 (d, J=1.7, 1H), 7.35 (t, J=7.9, 1H), 7.29-7.21 (m, 1H), 7.17-7.13 (m, 1H), 7.06-7.03 (m, 1H), 6.96-6.89 (m, 2H), 6.81-6.76 (m, 1H), 6.73 (d, J=1.7, 1H), 3.58-3.51 (m, 6H), 2.52-2.45 (m, 4H).

### Example 154

### 4-(3,4-Dibromobenzyl)-N-isoxazol-3-ylpiperazine-1-carboxamide

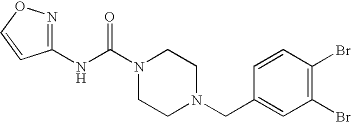

MS: 445.3. ¹H NMR (d₄-MeOH): 8.43 (d, J=1.8, 1H), 7.71 (d, J=1.9, 1H), 7.64 (d, J=8.2, 1H), 7.27-7.23 (dd, J=8.2, 2.0, 1H), 6.73 (d, J=1.8, 1H), 3.58-3.49 (m, 6H), 2.52-2.43 (m, 4H).

### Example 155

### N-Isoxazol-3-yl-4-(3-{4-[(trifluoromethyl)sulfanyl]phenoxy}benzyl)-piperazine-1-carboxamide

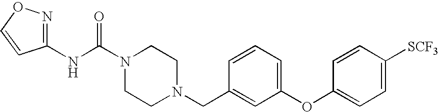

MS: 479.5. ¹H NMR (d₄-MeOH): 8.42 (d, J=1.8, 1H), 7.68-7.63 (m, 2H), 7.39 (t, J=7.9, 1H), 7.23-7.19 (m, 1H), 7.12-7.10 (m, 1H), 7.07-7.02 (m, 2H), 7.02-6.98 (m, 1H), 6.73 (d, J=1.8, 1H), 3.59-3.52 (m, 6H), 2.53-2.45 (m, 4H).

### Example 156

### 4-{3-[4-Fluoro-3-(trifluoromethyl)phenoxy]benzyl}-N-isoxazol-3-ylpiperazine-1-carboxamide

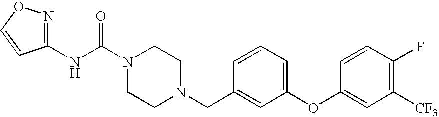

MS: 465.5. ¹H NMR (d₄-MeOH): 8.43 (d, J=1.8, 1H), 7.41-7.24 (m, 4H), 7.20-7.16 (m, 1H), 7.09-7.06 (m, 1H), 6.98-6.94 (m, 1H), 6.73 (d, J=1.8, 1H), 3.62-3.52 (m, 6H), 2.59-2.47 (m, 4H).

### Example 157

### 4-[3-(3-Bromophenoxy)benzyl]-N-isoxazol-3-ylpiperazine-1-carboxamide

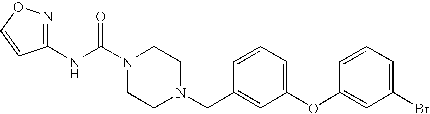

MS: 457.4. ¹H NMR (d₄-MeOH): 8.42 (d, J=1.7, 1H), 7.36 (t, J=7.9, 1H), 7.28-7.24 (m, 2H), 7.17-7.15 (m, 1H), 7.11-7.10 (m, 1H), 7.06-7.05 (m, 1H), 6.97-6.93 (m, 2H), 6.73 (d, J=1.7, 1H), 3.58-3.52 (m, 6H), 2.50-2.47 (m, 4H).

### Example 158

### N-Isoxazol-3-yl-4-(3-{4-[(trifluoromethyl)sulfonyl]phenoxy}benzyl)-piperazine-1-carboxamide

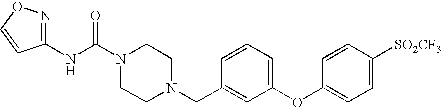

MS: 511.1. ¹H NMR (d₄-MeOH): 8.42 (d, J=1.8, 1H), 8.05-8.01 (m, 2H), 7.49-7.44 (m, 1H), 7.33-7.29 (m, 1H), 7.26-7.22 (m, 2H), 7.21-7.19 (m, 1H), 7.10-7.07 (m, 1H), 6.73 (d, J=1.8, 1H), 3.61 (s, 2H), 3.57-3.53 (m, 4H), 2.53-2.46 (m, 4H).

### Example 159

### N-Isoxazol-3-yl-4-{3-[3-(trifluoromethoxy)phenoxy]benzyl}piperazine-1-carboxamide

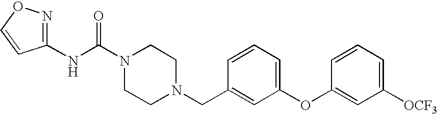

MS: 463.2. ¹H NMR (d₄-MeOH): 8.42 (d, J=1.8, 1H), 7.45-7.35 (m, 2H), 7.20-7.16 (m, 1H), 7.10-7.06 (m, 1H), 7.03-6.93 (m, 3H), 6.86-6.82 (m, 1H), 6.73 (d, J=1.8, 1H), 3.60-3.50 (m, 6H), 2.53-2.45 (m, 4H).

### Example 160

### 4-(3,4-Dichlorobenzyl)-N-isoxazol-3-ylpiperazine-1-carboxamide

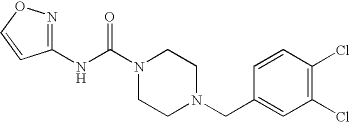

MS: 442.2. ¹H NMR (d₄-MeOH): 8.45 (d, J=1.6, 1H), 7.58-7.54 (m, 1H), 7.50 (d, J=8.2, 1H), 6.76-6.73 (m, 1H), 4.70-4.53 (m, 6H), 3.59-3.56 (m, 4H).

### Example 161

### N-isoxazol-3-yl-4-{3-[4-(trifluoromethyl)phenoxy]benzyl}piperazine-1-carboxamide

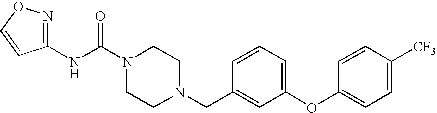

MS: 447.2. ¹H NMR (d₄-MeOH): 8.33-8.31 (dd, J=7.1, 2.1, 1H), 7.65-7.58 (m, 1H), 7.57-7.51 (m, 1H), 7.50-7.45 (m, 1H), 7.44-7.39 (m, 1H), 7.35-7.28 (m, 2H), 7.26-7.21 (m, 1H), 7.15-7.10 (m, 1H), 7.03-6.97 (m, 1H), 6.55-6.44 (m, 1H), 3.76-3.66 (m, 4H), 3.65-3.60 (m, 2H), 2.68-2.45 (m, 4H).

### Example 162

### N-Isoxazol-3-yl-4-[3-(quinolin-6-yloxy)benzyl]piperazine-1-carboxamide

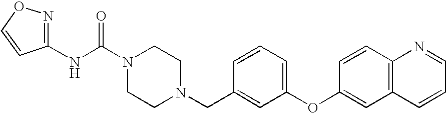

MS: 430.2. ¹H NMR (d₄-MeOH): 8.82-8.73 (m, 1H), 8.46-8.41 (m, 1H), 8.27-8.22 (m, 1H), 8.09-8.03 (m, 1H), 7.59-7.55 (dd, J=9.2, 2.7, 1H), 7.53-7.50 (dd, J=8.3, 4.3, 1H), 7.45-7.39 (m, 1H), 7.37-7.34 (m, 1H), 7.22 (d, J=7.6, 1H), 7.18-7.15 (m, 1H), 7.08-7.04 (m, 1H), 6.74 (d, J=1.8, 1H), 3.63-3.58 (m, 2H), 3.58-3.53 (m, 4H), 2.59-2.44 (m, 4H).

### Example 163

### 4-{3-[4-Cyano-3-(trifluoromethyl)phenoxy]benzyl}-N-isoxazol-3-ylpiperazine-1-carboxamide

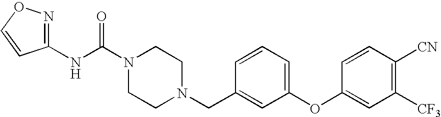

MS: 472.2. ¹H NMR (d₄-MeOH): 8.47-8.41 (m, 1H), 8.00-7.93 (m, 1H), 7.52-7.45 (m, 1H), 7.45-7.41 (m, 1H), 7.36-7.27 (m, 2H), 7.23-7.19 (m, 1H), 7.13-7.08 (m, 1H), 6.77-6.72 (m, 1H), 3.66-3.59 (m, 2H), 3.59-3.55 (m, 4H), 2.59-2.44 (m, 4H).

### Example 164

### 4-[3-(4-Chlorophenoxy)benzyl]-N-(5-methylisoxazol-3-yl)piperazine-1-carboxamide

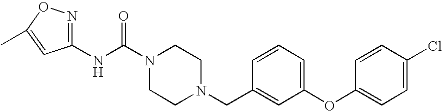

MS: 427.2. ¹H NMR (d₆-acetone): 9.01 (s, 1H), 7.51-7.44 (m, 1H), 7.42-7.36 (m, 3H), 7.32-7.28 (m, 1H), 7.14-7.10 (m, 1H), 7.08-7.03 (m, 2H), 6.52 (s, 1H), 4.48 (s, 2H), 4.22-3.69 (m, 4H), 3.49-3.29 (m, 4H), 2.34 (s, 3H).

### Example 165

### 4-(Quinolin-3-ylmethyl)-N-1H-tetrazol-5-ylpiperazine-1-carboxamide

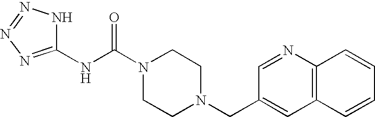

MS: 339.4. ¹H NMR (d₆-DMSO): 10.66 (s, 1H), 8.88 (d, J=2.1, 1H), 8.25 (d, J=1.3, 1H), 8.04-7.96 (m, 2H), 7.77-7.72 (m, 1H), 7.63-7.59 (m, 1H), 3.74 (s, 2H), 3.58-3.50 (m, 4H), 2.48-2.44 (m, 4H).

### Example 166

### 4-[3-(Naphthalen-2-yloxy)benzyl]-N-1H-tetrazol-5-ylpiperazine-1-carboxamide

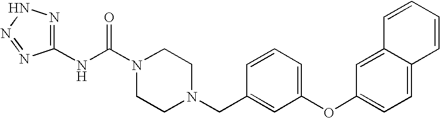

MS: 430.5. ¹H NMR (d₆-DMSO): 10.65 (s, 1H), 7.98 (d, J=8.9, 1H), 7.92 (d, J=7.9, 1H), 7.82 (d, J=8.0, 1H), 7.52-7.39 (m, 3H), 7.38 (d, J=7.9, 1H), 7.32-7.28 (dd, J=8.9, 2.5, 1H), 7.13 (d, J=7.7, 1H), 7.05 (s, 1H), 7.01-6.97 (dd, J=8.1, 1.8, 1H), 3.52 (s, 2H), 3.51-3.47 (m, 4H), 2.41-2.37 (m, 4H).

### Example 167

### 4-(3,4-Dibromobenzyl)-N-1H-tetrazol-5-ylpiperazine-1-carboxamide

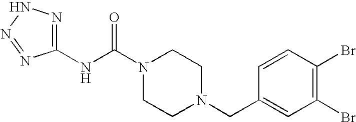

MS: 444.3. ¹H NMR (d₆-DMSO): 10.63 (s, 1H), 7.72 (d, J=8.2, 1H), 7.70 (d, J=1.9, 1H), 7.29-7.26 (dd, J=8.2, 1.9, 1H), 3.53-3.50 (m, 4H), 3.49 (s, 2H), 2.41-2.36 (m, 4H).

### Example 168

### 4-(4-Bromo-3-fluorobenzyl)-N-1H-tetrazol-5-ylpiperazine-1-carboxamide

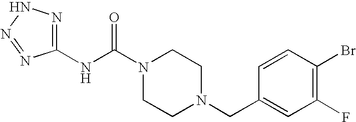

MS: 384.4. ¹H NMR (d₆-DMSO): 10.69 (s, 1H), 7.66 (t, J=7.8, 1H), 7.34-7.31 (dd, J=9.9, 1.6, 1H), 7.16-7.12 (dd, J=8.2, 1.5, 1H), 3.56-3.48 (m, 6H), 2.42-2.37 (m, 4H).

### Example 169

### 4-[3-(3,4-Difluorophenoxy)benzyl]-N-1H-tetrazol-5-ylpiperazine-1-carboxamide

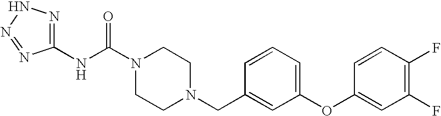

MS: 416.5. ¹H NMR (d₆-DMSO): 10.37 (s, 1H), 7.50-7.42 (dd, J=19.5, 9.3, 1H), 7.37 (t, J=7.9, 1H), 7.24-7.16 (m, 1H), 7.13 (d, J=7.6, 1H), 7.00 (s, 1H), 6.95-6.91 (m, 1H), 6.88-6.83 (m, 1H), 3.54-3.46 (m, 6H), 2.41-2.34 (m, 4H).

### Example 170

### 4-{3-[4-Cyano-3-(trifluoromethyl)phenoxy]benzyl}-N-1H-tetrazol-5-ylpiperazine-1-carboxamide

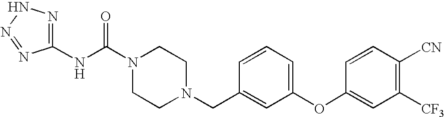

MS: 473.5. ¹H NMR (d₆-DMSO): 8.14 (d, J=8.7, 1H), 7.53 (s, 1H), 7.48 (t, J=7.8, 1H), 7.33 (d, J=7.6, 1H), 7.28 (d, J=7.4, 1H), 7.17 (s, 1H), 7.13 (d, J=8.2, 1H), 3.60-3.46 (m, 6H), 2.43-2.36 (m, 4H).

### Example 171

### N-1H-Tetrazol-5-yl-4-{3-[4-(trifluoromethyl)phenoxy]benzyl}piperazine-1-carboxamide

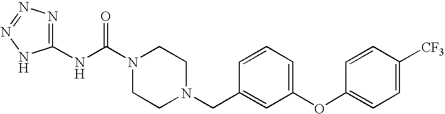

MS: 448.5. ¹H NMR (d₆-DMSO): 15.39 (s, 1H), 10.64 (s, 1H), 7.74 (d, J=8.6, 2H), 7.45-7.40 (m, 1H), 7.21 (d, J=7.6, 1H), 7.15 (d, J=8.6, 2H), 7.10-7.08 (m, 1H), 7.05-7.02 (m, 1H), 3.56-3.48 (m, 6H), 2.43-2.35 (m, 4H).

### Example 172

### N-1H-Tetrazol-5-yl-4-(3-{4-[(trifluoromethyl)sulfanyl]phenoxy}benzyl)-piperazine-1-carboxamide

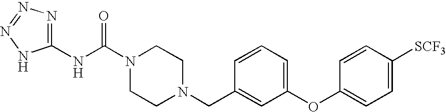

MS: 480.5. ¹H NMR (d₆-DMSO): 15.35 (s, 1H), 10.48 (s, 1H), 7.72 (d, J=8.7, 2H), 7.45-7.40 (m, 1H), 7.20 (d, J=7.6, 1H), 7.12-7.08 (m, 3H), 7.04-7.01 (m, 1H), 3.56-3.46 (m, 6H), 2.42-2.35 (m, 4H).

### Example 173

### N-1H-Tetrazol-5-yl-4-{3-[3-(trifluoromethoxy)phenoxy]benzyl}piperazine-1-carboxamide

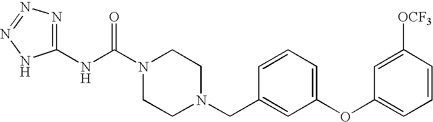

MS: 464.5. ¹H NMR (d₆-DMSO): 15.37 (s, 1H), 10.60 (s, 1H), 7.54-7.48 (m, 1H), 7.42-7.38 (m, 1H), 7.17 (d, J=7.6, 1H), 7.13 (d, J=8.3, 1H), 7.06-6.97 (m, 4H), 3.55-3.46 (m, 6H), 2.40-2.36 (m, 4H).

### Example 174

### 4-[3-(3,4-Dichlorophenoxy)benzyl]-N-1H-tetrazol-5-ylpiperazine-1-carboxamide

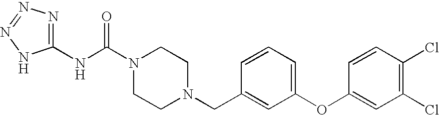

MS: 448.4. ¹H NMR (d₆-DMSO): 15.44 (s, 1H), 10.77 (s, 1H), 7.64 (d, J=8.9, 1H), 7.46-7.39 (m, 1H), 7.31 (s, 1H), 7.22-7.17 (m, 1H), 7.12-6.99 (m, 3H), 3.73-3.36 (m, 6H), 2.47-2.25 (m, 4H).

### Example 175

### 4-(Quinolin-2-ylmethyl)-N-1H-tetrazol-5-ylpiperazine-1-carboxamide

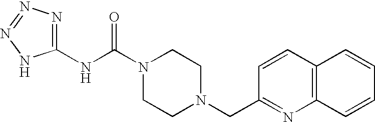

MS: 339.4. ¹H NMR (d₆-DMSO): 15.40 (s, 1H), 10.72 (s, 1H), 8.35 (d, J=8.5, 1H), 8.00-7.95 (m, 2H), 7.77-7.72 (m, 1H), 7.67 (d, J=8.5, 1H), 7.61-7.57 (m, 1H), 3.82 (s, 2H), 3.60-3.52 (m, 4H), 2.50-2.46 (m, 4H).

### Example 176

### 4-(Naphthalen-2-ylmethyl)-N-1H-tetrazol-5-ylpiperazine-1-carboxamide

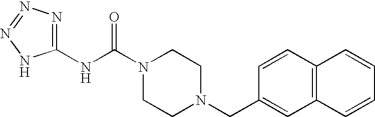

MS: 338.4. ¹H NMR (d₆-DMSO): 10.57 (s, 1H), 7.93-7.87 (m, 3H), 7.81 (s, 1H), 7.53-7.46 (m, 3H), 3.67 (s, 2H), 3.57-3.50 (m, 4H), 2.46-2.39 (m, 4H).

### Example 177

### 4-(4-Bromo-benzyl)-piperazine-1-carboxylic acid (2H-tetrazol-5-yl)-amide

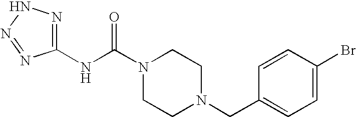

MS: 366.3. ¹H NMR (d₆-DMSO): 7.52 (d, J=8.4, 2H), 7.28 (d, J=8.4, 2H), 3.51 (t, J=4.8, 4H), 3.48 (s, 2H), 2.37 (t, J=4.8, 4).

### Example 178

### 4-(1H-Indol-6-ylmethyl)-piperazine-1-carboxylic acid (2H-tetrazol-5-yl)-amide

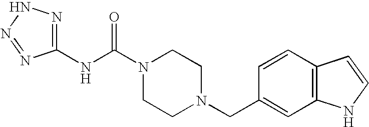

MS: 327.4. ¹H NMR (d₆-DMSO): 11.02 (s, 1H), 10.59 (br s, 1H), 7.44 (s, 1H), 7.34-7.30 (m, 2H), 7.05 (d, J=8.4, 1H), 6.38 (s, 1H), 3.56 (s, 2H), 3.50 (br s, 4H), 2.39 (br t, J=5.4, 4H).

### Example 179

### 4-(3-Benzyloxy-benzyl)-piperazine-1-carboxylic acid (2H-tetrazol-5-yl)-amide

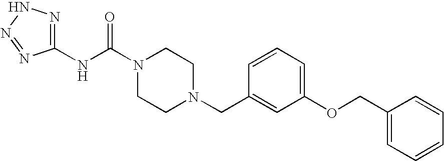

MS: 394.5. ¹H NMR (d₆-DMSO): 15.35 (br s, 1H), 10.65 (s, 1H), 7.45 (d, J=7.8, 2H), 7.39 (t, J=7.8, 2H), 7.32 (t, J=7.2, 1H), 7.24 (t, J=7.8, 1H), 6.96 (m, 1H), 6.92-6.88 (m, 2H), 5.10 (s, 2H), 3.50-3.48 (m, 6H), 3.33 (s, 2H), 2.36 (t, J=4.8, 4H).

### Example 180

### 4-Benzo[1,3]dioxol-5-ylmethyl-piperazine-1-carboxylic acid (2H-tetrazol-5-yl)-amide

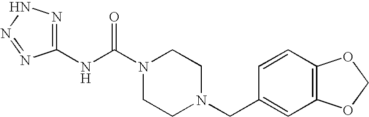

MS: 332.4. ¹H NMR (d₆-DMSO): 15.34 (br s, 1H), 10.66 (s, 1H), 6.87 (s, 1H), 6.85 (d, J=7.8, 1H), 6.75 (d, J=7.2, 1H), 5.99 (s, 2H), 3.50 (br s, 4H), 3.41 (s, 2H), 2.36 (t, J=4.8, 4H).

### Example 181

### 4-(3-Phenoxy-benzyl)-piperazine-1-carboxylic acid (2H-tetrazol-5-yl)-amide

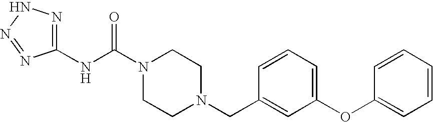

MS: 380.5. ¹H NMR (d₆-DMSO): 10.23 (br s, 1H), 7.39 (t, J=7.8, 2H), 7.34 (t, J=7.8, 1H), 7.14 (t, J=7.2, 1H), 7.08 (d, J=7.2, 1H), 7.01 (d, J=7.8, 2H), 6.98 (s, 1H), 6.90-6.88 (m, 1H), 3.50-3.48 (m, 6H), 2.37 (br s, 4H).

### Example 182

### 4-(3,4-Dichloro-benzyl)-piperazine-1-carboxylic acid (2H-tetrazol-5-yl)-amide

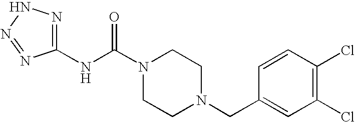

¹H NMR (d₆-DMSO): 10.56 (br s, 1H), 7.60-7.57 (m, 2H), 7.33-7.32 (dd, J=1.8, 8.4, 1H), 3.52-3.51 (m, 6H), 2.39 (t, J=4.8, 4H).

### Example 183

### 4-Benzo[b]thiophen-2-ylmethyl-piperazine-1-carboxylic acid (2H-tetrazol-5-yl)-amide

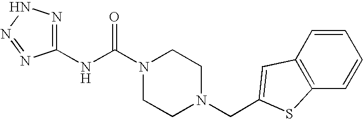

MS: 344.4. ¹H NMR (d₆-DMSO): 15.22 (br s, 1H), 10.67 (br s, 1H), 7.89 (d, J=7.8, 1H), 7.76 (d, J=7.2, 1H), 7.35-7.29 (m, 3H), 3.83 (s, 2H), 3.54 (t, J=4.8, 4H), 2.52-2.48 (m, 4H).

### Example 184

### 4-[3-(3-Cyano-phenoxy)-benzyl]-piperazine-1-carboxylic acid (2H-tetrazol-5-yl)-amide

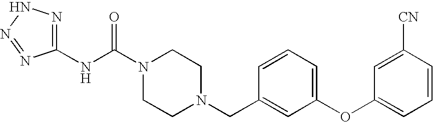

MS: 405.5. ¹H NMR (d₆-DMSO): 15.38 (br s, 1H), 10.67 (s, 1H), 7.61-7.57 (m, 2H), 7.49 (t, J=1.2, 1H), 7.40 (t, J=7.8, 1H), 7.35-7.33 (m, 1H), 7.17 (d, J=7.8, 1H), 7.04 (br s, 1H), 6.98-6.97 (dd, J=1.8, 8.4, 1H), 3.53 (s, 2H), 3.51 (br t, J=4.2, 4H), 2.39 (t, J=4.8, 4H).

### Example 185

### 4-{3-[4-Fluoro-3-(trifluoromethyl)phenoxy]benzyl}-N-2H-tetrazol-5-ylpiperazine-1-carboxamide

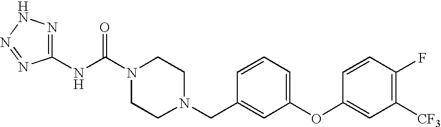

MS: 466.2. ¹H NMR (d₆-DMSO): 10.45-9.75 (m, 1H), 7.50-7.24 (m, 1H), 7.22-7.14 (m, 3H), 6.98-6.92 (m, 1H), 6.85-6.82 (m, 1H), 6.79-6.73 (m, 1H), 3.37-3.21 (m, 6H), 2.23-2.09 (m, 4H).

### Example 186

### N-2H-Tetrazol-5-yl-4-{3-[3-(trifluoromethyl)phenoxy]benzyl}piperazine-1-carboxamide

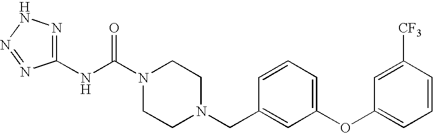

MS: 448.2. ¹H NMR (d₆-DMSO): 10.84-10.43 (m, 1H), 7.66-7.59 (m, 1H), 7.52-7.47 (m, 1H), 7.43-7.38 (m, 1H), 7.34-7.27 (m, 2H), 7.20-7.15 (m, 1H), 7.08-7.04 (m, 1H), 7.02-6.98 (m, 1H), 3.58-3.45 (m, 6H), 2.45-2.30 (m, 4H).

### Example 187

### 4-[3-(4-Cyanophenoxy)benzyl]-N-2H-tetrazol-5-ylpiperazine-1-carboxamide

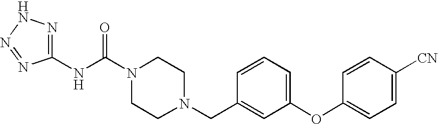

MS: 405.2. ¹H NMR (d₆-DMSO): 10.84-10.41 (m, 1H), 7.95-7.67 (m, 2H), 7.48-7.38 (m, 2H), 7.26-7.19 (m, 1H), 7.13-7.08 (m, 2H), 7.07-7.03 (m, 1H), 3.62-3.41 (m, 6H), 2.43-2.31 (m, 4H).

### Example 188

### N-2H-Tetrazol-5-yl-4-{3-[4-(2,2,2-trifluoroethoxy)phenoxy]benzyl}-piperazine-1-carboxamide

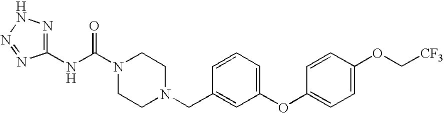

MS: 478.2. ¹H NMR (d₆-DMSO): 10.76-10.52 (m, 1H), 7.35-7.28 (m, 1H), 7.13-7.07 (m, 2H), 7.07-7.01 (m, 3H), 6.94-6.90 (m, 1H), 6.85-6.80 (m, 1H), 4.76-4.74 (q, J=8.9, 2H), 3.58-3.41 (m, 6H), 2.44-2.25 (m, 4H).

### Example 189

### 4-{3-[(2,2-Difluoro-1,3-benzodioxol-5-yl)oxy]benzyl}-N-2H-tetrazol-5-ylpiperazine-1-carboxamide

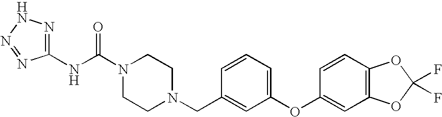

MS: 460.2. ¹H NMR (d₆-DMSO): 10.84-10.18 (m, 1H), 7.42 (d, J=8.8, 1H), 7.37-7.32 (m, 1H), 7.26 (d, J=2.4, 1H), 7.13-7.08 (m, 1H), 7.01-6.96 (m, 1H), 6.92-6.87 (m, 1H), 6.87-6.83 (dd, J=8.8, 2.4, 1H), 3.60-3.43 (m, 6H), 2.44-2.30 (m, 4H).

### Example 190

### 4-[3-(2-Chlorophenoxy)benzyl]-N-2H-tetrazol-5-ylpiperazine-1-carboxamide

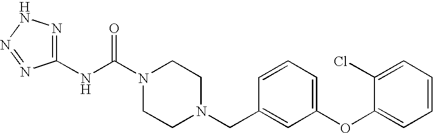

MS: 414.2. ¹H NMR (d₆-DMSO): 10.87-10.51 (m, 1H), 7.62-7.59 (dd, J=8.0, 1.6, 1H), 7.41-7.30 (m, 2H), 7.25-7.21 (dt, J=7.7, 1.5, 1H), 7.13-7.07 (m, 2H), 6.95-6.91 (m, 1H), 6.86-6.82 (m, 1H), 3.54-3.48 (m, 6H), 2.44-2.30 (m, 4H).

### Example 191

### 4-[3-(4-Chloro-phenoxy)-benzyl]-piperazine-1-carboxylic acid (1,5-dimethyl-1H-pyrazol-3-yl)-amide trifluoroacetic acid salt

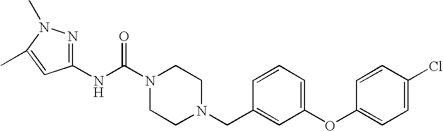

MS: 440.2. ¹H NMR (d₆-acetone): 10.03 (s, 1H), 7.49, (t, J=7.8, 1H), 7.41-7.38 (m, 3H), 7.31 (t, J=1.8, 1H), 7.14-7.12 (m, 1H), 7.08-7.05 (m, 2H), 6.46 (d, J=2.4, 1H), 4.50 (s, 2H), 3.80 (d, J=1.8, 3H), 4.55-3.05 (br hump, 8H), 2.36 (s, 3H).

### Example 192

### 4-[3-(4-Chloro-phenoxy)-benzyl]-piperazine-1-carboxylic acid (4-bromo-1-methyl-1H-pyrazol-3-yl)-amide trifluoroacetic acid salt

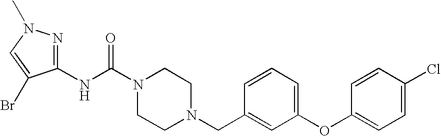

MS: 504.1. ¹H NMR (d₆-acetone): 7.66 (s, 1H), 7.50 (t, J=7.8, 1H), 7.41-7.39 (m, 3H), 7.31 (t, J=2.4, 1H), 7.15-7.13 (dd, J=2.4, 7.8, 1H), 7.08-7.06 (m, 2H), 4.52 (s, 2H), 4.33 (br hump, 2H), 3.79 (s, 3H), 3.52 (br hump, 6H).

### Example 193

### 4-[3-(4-Chloro-phenoxy)-benzyl]-piperazine-1-carboxylic acid (2-ethyl-2H-pyrazol-3-yl)-amide trifluoroacetic acid salt

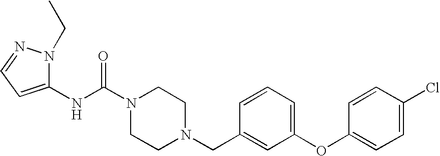

MS: 440.2. ¹H NMR (d₆-acetone): 7.51-7.48 (m, 2H), 7.41-7.39 (m, 3H), 7.30 (t, J=2.4, 1H), 7.15-7.13 (m, 1H), 7.08-7.06 (m, 2H), 4.53 (s, 2H), 4.30 (br hump, 2H), 4.09-4.05 (m, 2H), 3.51 (br hump, 6H), 1.34 (t, J=7.2, 3H).

### Example 194

### 4-[3-(4-Chlorophenoxy)benzyl]-N-(5-methyl-1H-pyrazol-3-yl)piperazine-1-carboxamide

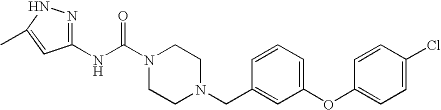

MS: 426.2. ¹H NMR (d₆-acetone): 7.51-7.38 (m, 5H), 7.35-7.31 (m, 1H), 7.15-7.11 (m, 1H), 7.09-7.06 (m, 2H), 4.48 (s, 2H), 4.24-3.66 (m, 4H), 3.50-3.35 (m, 4H), 2.32 (s, 3H).

### Example 195

### 4-(3,4-Dibromobenzyl)-N-pyridazin-3-ylpiperazine-1-carboxamide

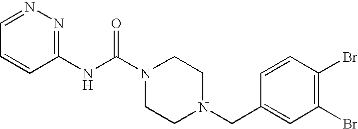

MS: 454.2. ¹H NMR (d₄-MeOH): 8.81-8.76 (m, 1H), 8.11 (d, J=9.0, 1H), 7.71 (d, J=1.9, 1H), 7.64 (d, J=8.2, 1H), 7.60-7.57 (m, 1H), 7.27-7.24 (dd, J=8.2, 1.9, 1H), 3.63-3.59 (m, 4H), 3.53 (s, 2H), 2.54-2.47 (m, 4H).

### Example 196

### 4-[(2,2-Difluoro-1,3-benzodioxol-5-yl)methyl]-N-pyridazin-3-ylpiperazine-1-carboxamide

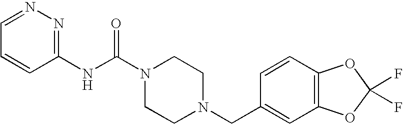

MS: 378.2. ¹H NMR (d₄-MeOH): 8.81-8.76 (m, 1H), 8.11 (d, J=8.8, 1H), 7.60-7.57 (dd, J=9.1, 4.7, 1H), 7.27-7.24 (m, 1H), 7.15-7.13 (m, 2H), 3.63-3.59 (m, 4H), 3.58 (s, 2H), 2.53-2.49 (m, 4H).

### Example 197

### N-Pyridazin-3-yl-4-(quinolin-3-ylmethyl)piperazine-1-carboxamide

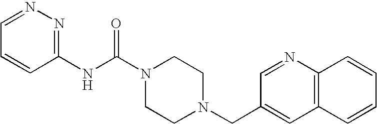

MS: 349.2. ¹H NMR (d₄-MeOH): 8.90-8.87 (m, 1H), 8.80-8.77 (m, 1H), 8.31-8.29 (m, 1H), 8.13-8.09 (m, 1H), 8.04 (d, J=8.5, 1H), 7.95 (d, J=8.1, 1H), 7.79-7.75 (m, 1H), 7.65-7.61 (m, 1H), 7.60-7.57 (m, 1H), 3.81 (s, 2H), 3.66-3.62 (m, 4H), 2.62-2.57 (m, 4H).

### Example 198

### N-Pyridazin-3-yl-4-(quinolin-2-ylmethyl)piperazine-1-carboxamide

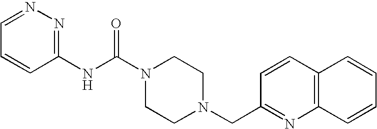

MS: 349.2. ¹H NMR (d₄-MeOH): 8.81-8.77 (m, 1H), 8.34 (d, J=8.5, 1H), 8.12 (d, J=9.1, 1H), 8.03 (d, J=8.5, 1H), 7.94-7.91 (m, 1H), 7.78-7.73 (m, 2H), 7.61-7.56 (m, 2H), 3.89 (s, 2H), 3.67-3.62 (m, 4H), 2.66-2.59 (m, 4H).

### Example 199

### 4-(3,4-Dichlorobenzyl)-N-pyridazin-3-ylpiperazine-1-carboxamide

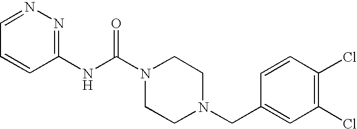

MS: 366.1. ¹H NMR (d₄-MeOH): 8.82-8.76 (m, 1H), 8.15-8.07 (m, 1H), 7.61-7.58 (dd, J=9.1, 4.7, 1H), 7.56 (d, J=1.9, 1H), 7.48 (d, J=8.2, 1H), 7.32-7.28 (dd, J=8.2, 2.0, 1H), 3.64-3.59 (m, 4H), 3.55 (s, 2H), 2.56-2.47 (m, 4H).

### Example 200

### 4-(Naphthalen-2-ylmethyl)-N-pyridazin-3-ylpiperazine-1-carboxamide

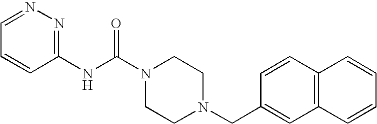

MS: 348.4. ¹H NMR (d₄-MeOH): 8.80-8.76 (m, 1H), 8.10 (d, J=9.0, 1H), 7.87-7.81 (m, 3H), 7.80-7.78 (m, 1H), 7.61-7.57 (dd, J=9.1, 4.7, 1H), 7.55-7.52 (dd, J=8.5, 1.6, 1H), 7.50-7.43 (m, 2H), 3.74 (s, 2H), 3.65-3.59 (m, 4H), 2.61-2.53 (m, 4H).

### Example 201

### 4-(1H-Indol-5-ylmethyl)-N-pyridazin-3-ylpiperazine-1-carboxamide

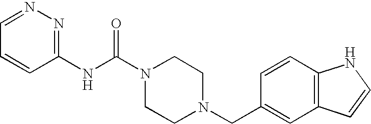

MS: 337.2. ¹H NMR (d₄-MeOH): 8.80-8.75 (m, 1H), 8.10 (d, J=9.1, 1H), 7.60-7.55 (m, 1H), 7.52-7.50 (m, 1H), 7.37-7.34 (m, 1H), 7.23-7.20 (m, 1H), 7.13-7.10 (m, 1H), 6.43-6.40 (m, 1H), 3.65 (s, 2H), 3.62-3.58 (m, 4H), 2.58-2.52 (m, 4H).

### Example 202

### N-2,1,3-Benzothiadiazol-4-yl-4-{[3-(phenylethynyl)phenyl]methyl}-piperazine-1-carboxamide

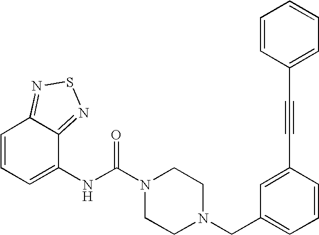

MS: 454.2. ¹H NMR (CDCl₃): 8.28-8.22 (m, 1H), 7.88 (s, 1H), 7.59-7.56 (m, 2H), 7.55-7.51 (m, 3H), 7.47-7.44 (m, 1H), 7.38-7.30 (m, 5H), 3.67-3.62 (m, 4H), 3.57 (s, 2H), 2.61-2.52 (m, 4H).

### Example 203

### N-2,1,3-Benzoxadiazol-4-yl-4-{[3-(phenylethynyl)phenyl]methyl}-piperazine-1-carboxamide

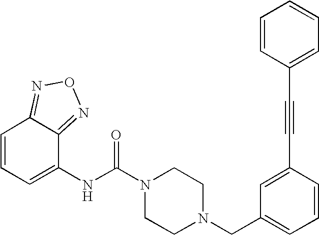

MS: 438.2. ¹H NMR (CDCl₃): 8.07-8.01 (m, 1H), 7.60-7.51 (m, 3H), 7.50-7.45 (m, 1H), 7.44-7.31 (m, 7H), 3.72-3.51 (m, 6H), 2.66-2.46 (m, 4H).

The compounds in Examples 204-209 were prepared using methods analogous to those described in Example 28.

### Example 204

### 4-[3-(3-Chloro-4-trifluoromethyl-phenoxy)-benzyl]-piperazine-1-carboxylic acid benzo[d]isoxazol-3-ylamide trifluoroacetic acid salt

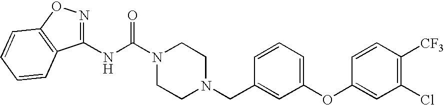

¹H NMR (CDCl₃): 9.68 (s, 1H), 7.92 (d, J=8.0, 1H), 7.51 (t, J=7.0, 1H), 7.42 (t, J=8.0, 1H), 7.36 (d, J=8.5, 1H), 7.27-7.22 (m, 2H), 7.12-7.10 (m, 2H), 7.05 (d, J=2.5, 1H), 6.90-6.88 (dd, J=2.5, 8.5, 1H), 4.18 (s, 2H), 3.93 (br hump, 4H), 3.22 (br hump, 4H).

### Example 205

### 4-[3-(4-Chloro-3-trifluoromethyl-phenoxy)-benzyl]-piperazine-1-carboxylic acid benzo[d]isoxazol-3-ylamide trifluoroacetic acid salt

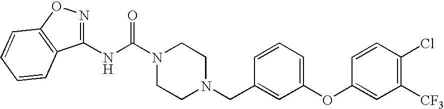

¹H NMR (CDCl₃): 9.62 (s, 1H), 7.94 (d, J=8.5, 1H), 7.54-7.51 (m, 1H), 7.44 (d, J=8.5, 1H), 7.41-7.37 (m, 2H), 7.30-7.26 (m, 2H), 7.17 (d, J=7.5, 1H), 7.08-7.04 (m, 3H), 4.18 (s, 2H), 3.93 (br hump, 4H), 3.21 (br hump, 4H).

### Example 206

### 4-[3-(4-Chloro-3-fluoro-phenoxy)-benzyl]-piperazine-1-carboxylic acid benzo[d]isoxazol-3-ylamide

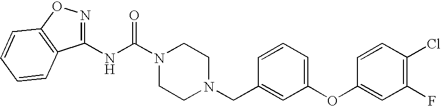

¹H NMR (CDCl₃): 8.24 (s, 1H), 8.08 (d, J=8.5, 1H), 7.55-7.52 (m, 1H), 7.45 (d, J=8.5, 1H), 7.36-7.29 (m, 3H), 7.16 (d, J=7.5, 1H), 7.07 (s, 1H), 6.96-6.94 (dd, J=2.0, 7.5, 1H), 6.82-6.79 (dd, J=2.5, 10.0, 1H), 6.77-6.75 (m, 1H), 3.66 (t, J=5.0, 4H), 3.57 (s, 2H), 2.56 (t, J=5.0, 4H).

### Example 207

### 4-[3-(3-Chloro-4-fluoro-phenoxy)-benzyl]-piperazine-1-carboxylic acid benzo[d]isoxazol-3-ylamide

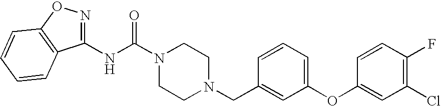

¹H NMR (CDCl₃): 8.67 (s, 1H), 8.08 (d, J=8.0, 1 H), 7.55-7.52 (m, 1H), 7.44 (d, J=8.0, 1H), 7.32-7.28 (m, 2H), 7.08-7.04 (m, 4H), 6.92-6.88 (m, 2 H), 3.67 (t, J=4.5, 4H), 3.56 (s, 2H), 2.56 (t, J=5.0, 4H).

### Example 208

### 4-[3-(4-Fluoro-phenoxy)-benzyl]-piperazine-1-carboxylic acid benzo[d]isoxazol-3-ylamide

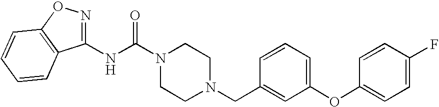

¹H NMR (CDCl₃): 8.69 (s, 1H), 8.08 (d, J=8.0, 1H), 7.55-7.51 (m, 1H), 7.44 (d, J=8.5, 1H), 7.31-7.27 (m, 2H), 7.04-6.98 (m, 6H), 6.89-6.87 (dd, J=2.0, 8.0, 1H), 3.68 (br s, 4H), 3.55 (s, 2H), 2.55 (br s, 4H).

### Example 209

### 4-[3-(4-Butyl-phenoxy)-benzyl]-piperazine-1-carboxylic acid benzo[d]isoxazol-3-ylamide

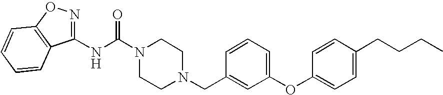

¹H NMR (CDCl₃): 8.53 (br s, 1H), 8.08 (d, J=8.0, 1H), 7.54-7.51 (m, 1H), 7.45 (d, J=8.5, 1H), 7.29 (d, J=8.0, 1H), 7.15 (d, J=8.5, 2H), 7.07 (br d, J=7.0, 1H), 7.04 (br s, 1H), 6.96-6.93 (m, 2H), 6.91-6.89 (dd, J=2.0, 8.0, 1H), 3.68 (br s, 4H), 3.56 (s, 2H), 2.61 (t, J=7.5, 2H), 2.56 (br s, 4H), 1.64-1.58 (m, 2H), 1.42-1.34 (m, 2H), 0.95 (t, J=7.5, 3H).

The compounds in Examples 210-244 were prepared using methods analogous to those described in Example 58.

### Example 210

### 4-[(2,2-Difluoro-1,3-benzodioxol-5-yl)methyl]-N-pyrazin-2-ylpiperazine-1-carboxamide

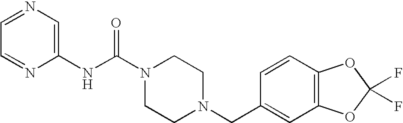

MS: 378.4. ¹H NMR (CDCl₃): 9.38 (d, J=1.5, 1H), 8.26 (d, J=2.6, 1H), 8.17-8.16 (dd, J=2.6, 1.6, 1H), 7.14 (s, 1H), 7.11 (s, 1H), 7.03-7.01 (m, 2H), 3.60-3.55 (m, 4H), 3.54 (s, 2H), 2.55-2.47 (m, 4H).

### Example 211

### 4-(1,3-Benzodioxol-5-ylmethyl)-N-pyrazin-2-ylpiperazine-1-carboxamide

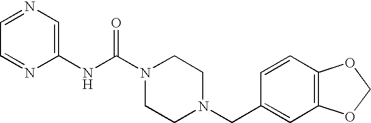

MS: 342.4. ¹H NMR (CDCl₃): 9.38 (d, J=1.5, 1H), 8.26 (d, J=2.6, 1H), 8.17-8.15 (dd, J=2.6, 1.6, 1H), 7.10 (s, 1H), 6.88 (d, J=0.9, 1H), 6.80-6.74 (m, 2H), 5.97 (s, 2H), 3.59-3.53 (m, 4H), 3.47 (s, 2H), 2.53-2.48 (m, 4H).

### Example 212

### 4-(4-Bromobenzyl)-N-pyrazin-2-ylpiperazine-1-carboxamide

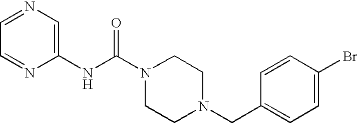

MS: 376.4. ¹H NMR (CDCl₃): 9.38 (d, J=1.5, 1H), 8.26 (d, J=2.6, 1H), 8.17-8.16 (dd, J=2.6, 1.6, 1H), 7.48 (d, J=8.4, 2H), 7.23 (d, J=8.4, 2H), 7.10 (s, 1H), 3.61-3.53 (m, 4H), 3.51 (s, 2H), 2.54-2.47 (m, 4H).

### Example 213

### 4-(Naphthalen-2-ylmethyl)-N-pyrazin-2-ylpiperazine-1-carboxamide

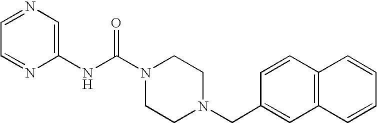

MS: 348.5. ¹H NMR (CDCl₃): 9.38 (d, J=1.4, 1H), 8.26 (d, J=2.6, 1H), 8.17-8.15 (dd, J=2.6, 1.6, 1H), 7.88-7.82 (m, 3H), 7.76 (s, 1H), 7.55-7.46 (m, 3H), 7.07 (s, 1H), 3.73 (s, 2H), 3.62-3.55 (m, 4H), 2.61-2.53 (m, 4H).

### Example 214

### N-Pyrazin-2-yl-4-{3-[4-(2,2,2-trifluoroethoxy)phenoxy]benzyl}-piperazine-1-carboxamide

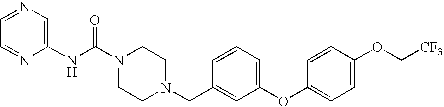

MS: 488.5. ¹H NMR (CDCl₃): 9.38 (d, J=1.4, 1H), 8.26 (d, J=2.6, 1H), 8.18-8.16 (dd, J=2.6, 1.6, 1H), 7.31-7.26 (m, 1H), 7.09-6.93 (m, 7H), 6.88-6.85 (dd, J=8.0, 2.1, 1H), 4.40-4.33 (q, J=8.1, 2H), 3.60-3.51 (m, 6H), 2.57-2.47 (m, 4H).

### Example 215

### N-Pyrazin-2-yl-4-{3-[4-(trifluoromethyl)phenoxy]benzyl}piperazine-1-carboxamide

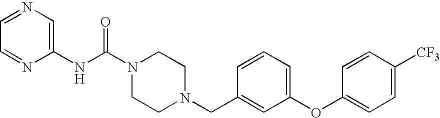

MS: 458.5. ¹H NMR (CDCl₃): 9.38 (d, J=1.5, 1H), 8.26 (d, J=2.6, 1H), 8.18-8.15 (dd, J=2.6, 1.6, 1H), 7.60 (d, J=8.9, 2H), 7.37 (t, J=7.8, 1H), 7.17 (d, J=7.6, 1H), 7.11-7.04 (m, 4H), 7.00-6.96 (dd, J=8.0, 1.7, 1H), 3.61-3.52 (m, 6H), 2.56-2.50 (m, 4H).

### Example 216

### 4-(1H-Indol-5-ylmethyl)-N-pyrazin-2-ylpiperazine-1-carboxamide

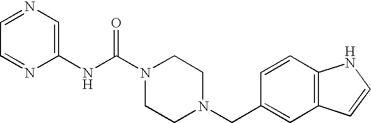

MS: 337.5. ¹H NMR (CDCl₃): 9.38 (d, J=1.5, 1H), 8.25 (d, J=2.6, 1H), 8.19 (s, 1H), 8.17-8.15 (dd, J=2.6, 1.6, 1H), 7.59 (s, 1H), 7.39 (d, J=8.3, 1H), 7.26-7.23 (m, 1H), 7.22-7.19 (dd, J=8.3, 1.5, 1H), 7.06 (s, 1H), 6.57-6.54 (m, 1H), 3.67 (s, 2H), 3.61-3.51 (m, 4H), 2.61-2.47 (m, 4H).

### Example 217

### 4-(3,4-Dibromobenzyl)-N-pyrazin-2-ylpiperazine-1-carboxamide

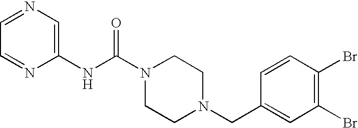

MS: 453.3. ¹H NMR (CDCl₃): 9.35 (d, J=1.5, 1H), 8.24 (d, J=2.6, 1H), 8.15-8.13 (m, 1H), 7.62 (d, J=2.0, 1H), 7.57 (d, J=8.2, 1H), 7.16-7.10 (m, 2H), 3.59-3.53 (m, 4H), 3.47 (s, 2H), 2.53-2.46 (m, 4H).

### Example 218

### 4-(1-Benzothiophen-2-ylmethyl)-N-pyrazin-2-ylpiperazine-1-carboxamide

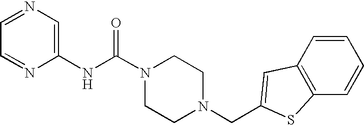

MS: 354.4. ¹H NMR (CDCl₃): 9.36 (d, J=1.5, 1H), 8.23 (d, J=2.6, 1H), 8.14-8.12 (m, 1H), 7.82-7.77 (m, 1H), 7.71-7.68 (m, 1H), 7.36-7.26 (m, 2H), 7.17-7.12 (m, 2H), 3.84 (br s, 2H), 3.62-3.54 (m, 4H), 2.64-2.56 (m, 4H).

### Example 219

### 4-[4-(Benzyloxy)benzyl]-N-pyrazin-2-ylpiperazine-1-carboxamide

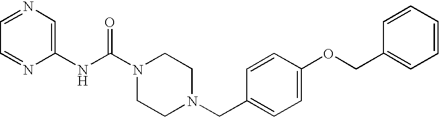

MS: 404.5. ¹H NMR (CDCl₃): 9.36 (d, J=1.5, 1H), 8.23 (d, J=2.6, 1H), 8.14-8.12 (m, 1H), 7.46-7.14 (m, 8H), 6.94 (d, J=8.7, 1H), 5.06 (s, 2H), 3.58-3.51 (m, 4H), 3.48 (s, 2H), 2.52-2.43 (m, 4H).

### Example 220

### 4-(3,4-Dichlorobenzyl)-N-pyrazin-2-ylpiperazine-1-carboxamide

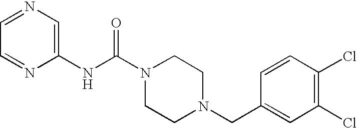

MS: 366.4. ¹H NMR (CDCl₃): 9.35 (d, J=1.5, 1H), 8.24 (d, J=2.6, 1H), 8.15-8.13 (m, 1H), 7.45 (d, J=2.0, 1H), 7.40 (d, J=8.2, 1H), 7.19-7.15 (m, 1H), 7.12 (s, 1H), 3.60-3.52 (m, 4H), 3.49 (s, 2H), 2.52-2.47 (m, 4H).

### Example 221

### 4-[3-(4-Bromophenoxy)benzyl]-N-pyrazin-2-ylpiperazine-1-carboxamide

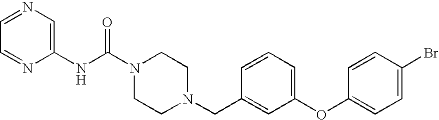

MS: 469.5. ¹H NMR (d₆-DMSO): 9.49 (s, 1H), 9.01 (d, J=1.5, 1H), 8.30-8.27 (m, 1H), 8.20 (d, J=2.6, 1H), 7.58-7.53 (m, 2H), 7.39-7.35 (m, 1H), 7.13 (d, J=7.6, 1H), 7.01-7.00 (m, 1H), 6.99-6.97 (m, 2H), 6.95-6.92 (m, 1H), 3.53-3.46 (m, 6H), 2.41-2.35 (m, 4H).

### Example 222

### 4-(4-Bromo-3-fluorobenzyl)-N-pyrazin-2-ylpiperazine-1-carboxamide

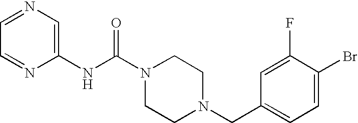

MS: 395.4. ¹H NMR (d₆-DMSO): 9.51 (s, 1H), 9.03 (d, J=1.5, 1H), 8.30-8.28 (m, 1H), 8.21 (d, J=2.6, 1H), 7.69-7.64 (m, 1H), 7.35-7.31 (m, 1H), 7.16-7.13 (m, 1H), 3.55-3.48 (m, 6H), 2.43-2.35 (m, 4H).

### Example 223

### 4-[3-(Benzyloxy)benzyl]-N-pyrazin-2-ylpiperazine-1-carboxamide

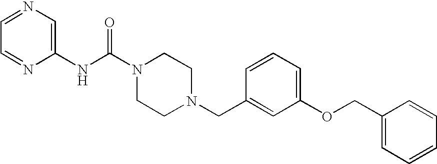

MS: 404.5. ¹H NMR (d₆-DMSO): 9.50 (s, 1H), 9.03 (d, J=1.5, 1H), 8.31-8.26 (m, 1H), 8.20 (d, J=2.6, 1H), 7.45 (d, J=7.0, 2H), 7.39 (t, J=7.4, 2H), 7.35-7.31 (m, 1H), 7.27-7.23 (m, 1H), 6.97 (s, 1H), 6.93-6.88 (m, 2H), 5.10 (s, 2H), 3.53-3.43 (m, 6H), 2.39-2.32 (m, 4H).

### Example 224

### N-Pyrazin-2-yl-4-(quinolin-3-ylmethyl)piperazine-1-carboxamide

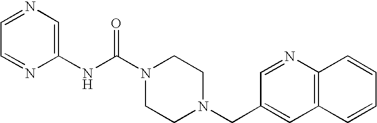

MS: 349.5. ¹H NMR (d₆-DMSO): 9.52 (s, 1H), 9.03 (d, J=1.5, 1H), 8.89 (d, J=2.1, 1H), 8.30-8.28 (m, 1H), 8.26-8.25 (m, 1H), 8.20 (d, J=2.6, 1H), 8.04-7.98 (m, 2H), 7.77-7.72 (m, 1H), 7.63-7.60 (m, 1H), 3.74 (s, 2H), 3.55-3.50 (m, 4H), 2.49-2.43 (m, 4H).

### Example 225

### 4-[3-(3-Chlorophenoxy)benzyl]-N-pyrazin-2-ylpiperazine-1-carboxamide

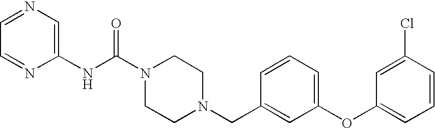

MS: 424.5. ¹H NMR (CDCl₃): 9.36 (d, J=1.5, 1H), 8.24 (d, J=2.6, 1H), 8.16-8.14 (m, 1H), 7.35-7.29 (m, 1H), 7.28-7.23 (m, 1H), 7.13-7.03 (m, 4H), 6.99-6.97 (m, 1H), 6.95-6.88 (m, 2H), 3.58-3.53 (m, 6H), 2.54-2.48 (m, 4H).

### Example 226

### N-Pyrazin-2-yl-4-(3-{4-[(trifluoromethyl)sulfonyl]phenoxy}benzyl)-piperazine-1-carboxamide

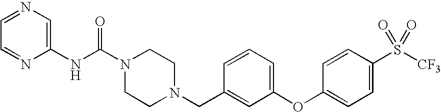

MS: 522.5. ¹H NMR (CDCl₃): 9.36 (d, J=1.5, 1H), 8.24 (d, J=2.6, 1H), 8.16-8.14 (m, 1H), 7.97 (d, J=8.9, 2H), 7.45-7.39 (m, 1H), 7.28-7.25 (m, 1H), 7.16-7.12 (m, 3H), 7.08 (s, 1H), 7.04-7.01 (m, 1H), 3.61-3.54 (m, 6H), 2.57-2.49 (m, 4H).

### Example 227

### 4-(3-Phenoxy-benzyl)-piperazine-1-carboxylic acid pyrazin-2-ylamide

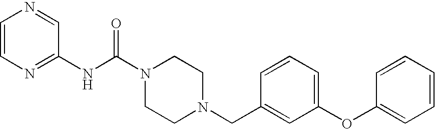

MS: 390.5. ¹H NMR (CDCl₃): 9.36 (d, J=1.8, 1H), 8.24 (d, J=2.4, 1H), 8.144-8.137 (m, 1H), 7.36-7.33 (m, 2H), 7.29 (t, J=7.8, 1H), 7.12-7.10 (m, 2H), 7.06 (d, J=7.8, 1H), 7.03-7.00 (m, 3H), 6.92-6.90 (m, 1H), 3.55 (t, J=4.8, 4H), 3.53 (s, 2H), 2.51 (t, J=4.8, 4H).

### Example 228

### 4-[3-(Naphthalen-2-yloxy)-benzyl]-piperazine-1-carboxylic acid pyrazin-2-ylamide

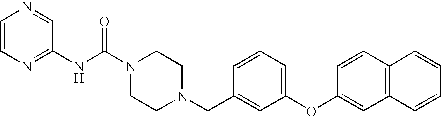

MS: 440.5. ¹H NMR (CDCl₃): 9.36 (d, J=1.8, 1H), 8.24 (d, J=5.0, 1H), 8.145-8.138 (dd, J=1.2, 3.0, 1H), 7.84 (t, J=8.4, 2H), 7.71 (d, J=7.2, 1H), 7.48-7.45 (m, 1H), 7.43-7.40 (m, 1H), 7.33-7.31 (m, 2H), 7.27-7.25 (m, 1H), 7.11-7.07 (m, 3H), 6.99-6.97 (m, 1H), 3.55-3.53 (m, 6H), 2.51 (t, J=4.8, 4H).

### Example 229

### 4-[3-(4-Cyano-phenoxy)-benzyl]-piperazine-1-carboxylic acid pyrazin-2-ylamide

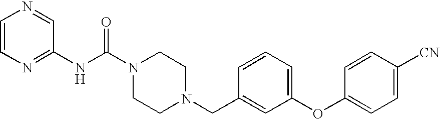

MS: 415.5. ¹H NMR (CDCl₃): 9.36 (d, J=1.8, 1H), 8.25 (d, J=2.4, 1H), 8.15-8.14 (m, 1H), 7.62-7.60 (m, 2H), 7.37 (t, J=7.8, 1H), 7.19 (d, J=7.8, 1H), 7.09-7.06 (m, 2H), 7.02-7.00 (m, 2H), 6.98-6.97 (dd, J=1.8, 7.2, 1H), 3.56-3.55 (m, 6H), 2.52 (t, J=4.8, 4H).

### Example 230

### 4-Benzofuran-2-ylmethyl-piperazine-1-carboxylic acid pyrazin-2-ylamide

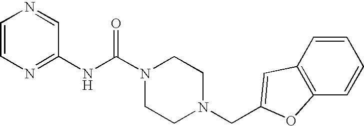

MS: 338.4. ¹H NMR (CDCl₃): 9.35 (s, 1H), 8.24 (d, J=3.0, 1H), 8.15-8.14 (dd, J=1.8, 2.4, 1H), 7.56-7.54 (m, 1H), 7.50-7.49 (m, 1H), 7.29-7.27 (m, 1H), 7.24-7.22 (m, 1H), 7.10 (s, 1H), 6.64 (s, 1H), 3.76 (s, 2H), 3.61 (t, J=4.8, 4H), 2.63 (t, J=4.8, 4H).

### Example 231

### 4-[3-(3,4-Difluorophenoxy)benzyl]-N-pyrazin-2-ylpiperazine-1-carboxamide

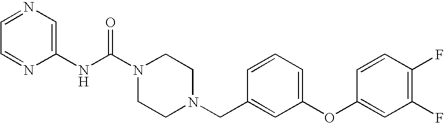

MS: 426.5. ¹H NMR (d₄-MeOH): 9.04-9.01 (m, 1H), 8.30-8.25 (m, 1H), 8.17-8.15 (m, 1H), 7.35 (t, J=7.9, 1H), 7.29-7.21 (m, 1H), 7.17-7.14 (m, 1H), 7.07-7.04 (m, 1H), 6.96-6.89 (m, 2H), 6.81-6.76 (m, 1H), 3.62-3.54 (m, 6H), 2.55-2.46 (m, 4H).

### Example 232

### N-Pyrazin-2-yl-4-[3-(quinolin-6-yloxy)benzyl]piperazine-1-carboxamide

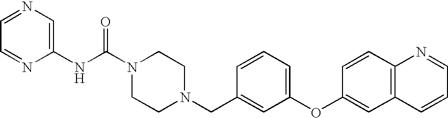

MS: 441.5. ¹H NMR (d₄-MeOH): 9.04-9.01 (m, 1H), 8.78-8.74 (m, 1H), 8.29-8.26 (m, 1H), 8.25-8.22 (m, 1H), 8.17-8.15 (m, 1H), 8.07-8.02 (m, 1H), 7.58-7.54 (m, 1H), 7.52-7.48 (m, 1H), 7.43-7.38 (m, 1H), 7.36-7.33 (m, 1H), 7.23-7.19 (m, 1H), 7.17-7.14 (m, 1H), 7.07-7.02 (m, 1H), 3.61-3.55 (m, 6H), 2.58-2.47 (m, 4H).

### Example 233

### N-Pyrazin-2-yl-4-{3-[4-(trifluoromethoxy)phenoxy]benzyl}piperazine-1-carboxamide

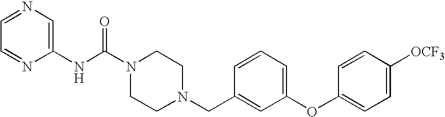

MS: 474.5. ¹H NMR (d₄-MeOH): 9.04-9.01 (m, 1H), 8.29-8.26 (m, 1H), 8.17-8.15 (m, 1H), 7.39-7.32 (m, 1H), 7.29-7.25 (m, 2H), 7.18-7.14 (m, 1H), 7.08-7.03 (m, 3H), 6.96-6.92 (m, 1H), 3.61-3.55 (m, 6H), 2.53-2.48 (m, 4H).

### Example 234

### N-Pyrazin-2-yl-4-(3-{4-[(trifluoromethyl)sulfanyl]phenoxy}benzyl)-piperazine-1-carboxamide

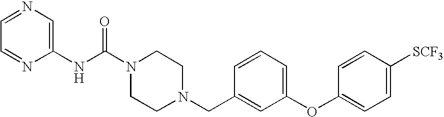

MS: 490.5. ¹H NMR (d₄-MeOH): 9.04-9.02 (m, 1H), 8.30-8.26 (m, 1H), 8.18-8.15 (m, 1H), 7.69-7.63 (m, 2H), 7.43-7.37 (m, 1H), 7.25-7.19 (m, 1H), 7.14-7.11 (m, 1H), 7.08-6.98 (m, 3H), 3.64-3.55 (m, 6H), 2.55-2.48 (m, 4H).

### Example 235

### 4-[3-(3-Cyanophenoxy)benzyl]-N-pyrazin-2-ylpiperazine-1-carboxamide

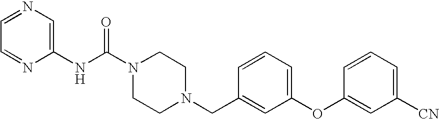

MS: 415.2. ¹H NMR (d₄-MeOH): 9.04-9.01 (m, 1H), 8.29-8.25 (m, 1H), 8.16-8.14 (m, 1H), 7.55-7.49 (m, 1H), 7.47-7.43 (m, 1H), 7.42-7.36 (m, 1H), 7.31-7.26 (m, 2H), 7.23-7.19 (m, 1H), 7.11-7.08 (m, 1H), 7.00-6.95 (m, 1H), 3.62-3.55 (m, 6H), 2.56-2.47 (m, 4H).

### Example 236

### 4-{3-[4-Cyano-3-(trifluoromethyl)phenoxy]benzyl}-N-Pyrazin-2-ylpiperazine-1-carboxamide

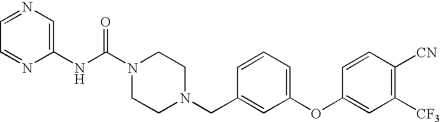

MS: 483.2. ¹H NMR (d₄-MeOH): 9.04-9.01 (m, 1H), 8.29-8.26 (m, 1H), 8.17-8.15 (m, 1H), 7.95-7.92 (m, 1H), 7.49-7.45 (m, 1H), 7.42-7.40 (m, 1H), 7.33-7.31 (m, 1H), 7.29-7.26 (m, 1H), 7.21-7.19 (m, 1H), 7.10-7.07 (m, 1H), 3.63-3.56 (m, 6H), 2.54-2.50 (m, 4H).

### Example 237

### 4-{3-[(2,2-Difluoro-1,3-benzodioxol-5-yl)oxy]benzyl}-N-pyrazin-2-ylpiperazine-1-carboxamide

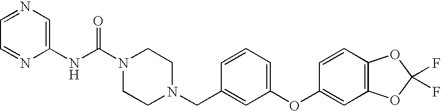

MS: 470.2. ¹H NMR (d₄-MeOH): 9.04-9.00 (m, 1H), 8.29-8.25 (m, 1H), 8.17-8.14 (m, 1H), 7.36-7.30 (m, 1H), 7.19-7.11 (m, 2H), 7.05-7.01 (m, 1H), 6.95-6.89 (m, 2H), 6.80-6.76 (m, 1H), 3.61-3.53 (m, 6H), 2.55-2.46 (m, 4H).

### Example 238

### 4-[3-(2-Chlorophenoxy)benzyl]-N-pyrazin-2-ylpiperazine-1-carboxamide

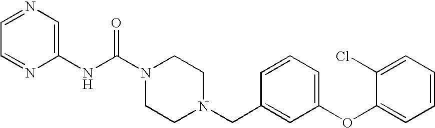

MS: 424.2. ¹H NMR (d₄-MeOH): 9.03-9.00 (m, 1H), 8.29-8.23 (m, 1H), 8.17-8.13 (m, 1H), 7.52-7.47 (m, 1H), 7.34-7.27 (m, 2H), 7.19-7.13 (m, 1H), 7.11-7.07 (m, 1H), 7.06-7.01 (m, 1H), 6.97-6.93 (m, 1H), 6.86-6.80 (m, 1H), 3.60-3.51 (m, 6H), 2.54-2.43 (m, 4H).

### Example 239

### N-Pyrazin-2-yl-4-(quinolin-2-ylmethyl)piperazine-1-carboxamide

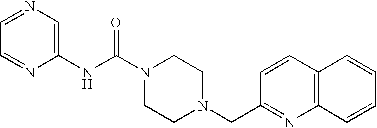

MS: 349.5. ¹H NMR (CDCl₃): 9.39 (d, J=1.5, 1H), 8.26 (d, J=2.6, 1H), 8.20-8.15 (m, 2H), 8.11 (d, J=8.4, 1H), 7.84 (d, J=8.0, 1H), 7.76-7.71 (m, 1H), 7.65 (d, J=8.4, 1H), 7.58-7.54 (m, 1H), 7.10 (s, 1H), 3.92 (s, 2H), 3.65-3.60 (m, 4H), 2.69-2.61 (m, 4H).

### Example 240

### 4-[3-(3-Bromophenoxy)benzyl]-N-pyrazin-2-ylpiperazine-1-carboxamide

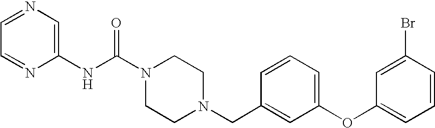

MS: 468.1. ¹H NMR (d₄-MeOH): 9.05 (d, J=1.5, 1H), 8.30-8.28 (dd, J=2.6, 1.6, 1H), 8.17 (d, J=2.6, 1H), 7.38 (t, J=7.9, 1H), 7.29-7.26 (m, 2H), 7.20-7.16 (m, 1H), 7.13-7.11 (m, 1H), 7.09-7.07 (m, 1H), 7.00-6.94 (m, 2H), 3.66-3.52 (m, 6H), 2.55-2.49 (m, 4H).

### Example 241

### 4-{3-[4-Fluoro-3-(trifluoromethyl)phenoxy]benzyl}-N-pyrazin-2-ylpiperazine-1-carboxamide

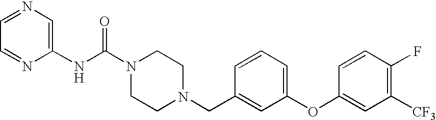

MS: 476.2. ¹H NMR (d₄-MeOH): 9.07-9.02 (m, 1H), 8.34-8.25 (m, 1H), 8.20-8.16 (m, 1H), 7.45-7.32 (m, 2H), 7.32-7.25 (m, 2H), 7.23-7.18 (m, 1H), 7.11-7.08 (m, 1H), 6.99-6.94 (m, 1H), 3.64-3.56 (m, 6H), 2.59-2.44 (m, 4H).

### Example 242

### N-Pyrazin-2-yl-4-{3-[3-(trifluoromethoxy)phenoxy]benzyl}piperazine-1-carboxamide

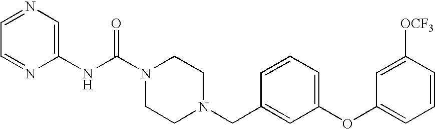

MS: 474.2. ¹H NMR (d₄-MeOH): 9.08-8.99 (m, 1H), 8.35-8.24 (m, 1H), 8.19-8.16 (m, 1H), 7.48-7.33 (m, 2H), 7.23-7.18 (m, 1H), 7.12-7.09 (m, 1H), 7.05-6.96 (m, 3H), 6.89-6.84 (m, 1H), 3.66-3.53 (m, 6H), 2.58-2.43 (m, 4H).

### Example 243

### 4-[3-(4-Chlorophenoxy)benzyl]-N-(3-chloropyrazin-2-yl)piperazine-1-carboxamide

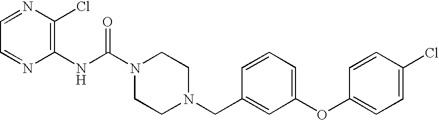

MS: 458.1. ¹H NMR (CDCl₃): 8.26 (s, 1H), 8.00 (s, 1H), 7.32-7.27 (m, 3H), 7.11-7.06 (m, 1H), 7.03-6.87 (m, 5H), 3.64-3.48 (m, 6H), 2.56-2.46 (m, 4H).

### Example 244

### 4-[3-(4-Chlorophenoxy)benzyl]-N-(5-phenyl-1H-pyrazol-3-yl)piperazine-1-carboxamide trifluoroacetate salt

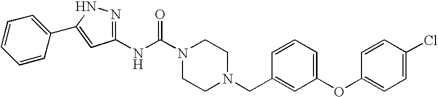

MS: 488.2. ¹H NMR (d₆-acetone): 7.96-7.78 (dd, J=1.8, 8.4, 2H), 7.50 (t, J=7.8, 1H), 7.45 (d, J=7.2, 1H), 7.41-7.34 (m, 7H), 7.15-7.13 (m, 1H), 7.08-7.06 (m, 2H), 4.58 (s, 2H), 3.62 (br hump, 8H).

The compounds in Examples 245-246 were prepared using methods analogous to those described for Example 1.

### Example 245

### 4-[3-(4-Chloro-phenoxy)-benzyl]-piperazine-1-carboxylic acid (6-fluoro-benzo[d]isoxazol-3-yl)-amide

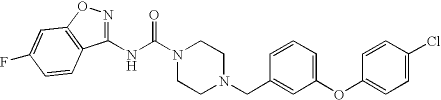

MS: 481.1. ¹H NMR (d₆-acetone): 8.02-7.56 (m, 2H), 7.49 (t, J=8.4, 1H), 7.43-7.34 (m, 4H), 7.31 (t, J=1.8, 1H), 7.14-7.11 (m, 2H), 7.06-7.04 (m, 2H), 4.51 (s, 2H), 3.49 (br hump, 8H).

### Example 246

### 4-[3-(4-Chloro-phenoxy)-benzyl]-piperazine-1-carboxylic acid pyridazin-3-ylamide

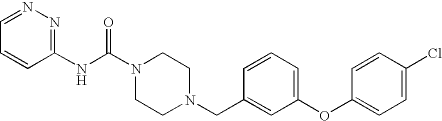

MS: 424.2. ¹H NMR (CDCl₃): 8.81 (s, 1H), 8.26 (s, 2H), 7.44-7.36 (m, 1H), 7.32-7.26 (m, 3H), 7.08 (d, J=7.6, 1H), 7.02-6.99 (m, 1H), 6.96-6.92 (m, 2H), 6.91-6.88 (dd, J=8.1, 2.4, 1H), 3.64-3.55 (m, 4H), 3.52 (s, 2H), 2.51-2.47 (m, 4H).

### Biological Testing:

### Assay Method 1

A. Transfection of Cells with Human FAAH

A 10-cm tissue culture dish with a confluent monolayer of SK—N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled human FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

### B. FAAH Assay

T84 frozen cell pellets or transfected SK—N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog number MAFCNOB50; Millipore, Bedford, Mass., USA) were loaded with 25 μL of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount.

### Assay Method 2

A. Transfection of Cells with Rat FAAH

A 10-cm tissue culture dish with a confluent monolayer of SK—N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled rat FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

### B. FAAH Assay

T84 frozen cell pellets or transfected SK—N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog number MAFCNOB50; Millipore, Bedford, Mass., USA) were loaded with 25 μL of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount.

Results for compounds tested in these assays are summarized in Table 1, as an average of results obtained. Compounds were tested in free base, hydrochloride salt, and/or trifluoroacetic acid salt forms. Where activity is shown as greater than (>) a particular value, the value is the solubility limit of the compound in the assay medium or the highest concentration tested in the assay.

TABLE 1

| | Assay 1 | Assay 2 |
|---|---|---|
| Ex. | IC₅₀ (μM) | IC₅₀ (μM) |
| 1 | 0.103 | 0.337 |
| 2 | 0.212 | 10.000 |
| 3 | 1.200 | 9.000 |
| 4 | 0.059 | 0.260 |
| 5 | 0.170 | 1.200 |
| 6 | 0.308 | 0.352 |
| 7 | 2.000 | 2.400 |
| 8 | 0.770 | 0.290 |
| 9 | 0.552 | 0.057 |
| 10 | 0.244 | 0.036 |
| 11 | 1.000 | 0.170 |
| 12 | 5.000 | 3.000 |
| 13 | 0.005 | 0.087 |
| 14 | 0.218 | 0.063 |
| 15 | 0.059 | 0.023 |
| 16 | 0.039 | 0.005 |
| 17 | 10.000 | 0.517 |
| 18 | >10 | 8.999 |
| 19 | 1.500 | 0.055 |
| 20 | 0.935 | 0.250 |
| 21 | 0.043 | 0.007 |
| 22 | 0.108 | 0.077 |
| 23 | 10.000 | 5.000 |
| 24 | 10.000 | 4.000 |
| 25 | 1.300 | 1.300 |
| 26 | 5.000 | 1.000 |
| 27 | 0.077 | 0.047 |
| 28 | 0.095 | 0.017 |
| 29 | 0.016 | 0.023 |
| 30 | 0.0001 | 0.014 |
| 31 | 0.022 | 0.017 |
| 32 | 0.003 | 0.004 |
| 33 | 0.052 | 0.030 |
| 34 | 0.152 | 0.190 |
| 35 | 0.005 | 0.020 |
| 36 | 0.008 | 0.010 |
| 37 | 0.032 | 0.006 |
| 38 | 0.070 | 0.009 |
| 39 | 0.310 | 0.573 |
| 40 | 0.156 | 0.015 |
| 41 | 0.048 | 0.141 |
| 42 | 0.311 | 0.018 |
| 43 | 0.983 | 0.006 |
| 44 | 0.643 | 0.046 |
| 45 | 1.500 | 0.410 |
| 46 | 0.615 | 0.025 |
| 47 | 1.145 | 0.066 |
| 48 | 0.627 | 0.049 |
| 49 | 2.000 | 0.360 |
| 50 | 0.042 | 0.018 |
| 51 | 0.028 | 0.029 |
| 52 | 0.074 | 0.322 |
| 53 | 0.210 | 0.030 |
| 54 | 0.370 | 0.050 |
| 55 | 7.746 | 0.764 |
| 56 | 1.500 | 0.550 |
| 57 | 0.068 | 0.167 |
| 58 | 0.084 | 0.009 |
| 59 | 0.270 | 0.022 |
| 60 | 0.166 | 0.016 |
| 61 | 2.300 | 7.000 |
| 62 | 0.041 | 0.035 |
| 63 | 0.070 | 0.077 |
| 64 | 0.105 | 0.080 |
| 65 | 0.395 | 0.300 |
| 66 | 0.024 | 0.046 |
| 67 | 0.006 | 0.052 |
| 68 | 0.016 | 0.315 |
| 69 | 0.044 | 2.000 |
| 70 | 0.046 | 0.046 |
| 71 | 0.013 | 0.050 |
| 72 | 0.024 | 0.030 |
| 73 | 0.024 | 0.033 |
| 74 | 0.410 | 0.325 |
| 75 | 0.123 | 0.110 |
| 76 | 0.010 | 0.400 |
| 77 | 0.012 | 0.064 |
| 78 | 0.059 | 0.410 |
| 79 | 0.046 | 0.320 |
| 80 | 5.000 | 4.000 |
| 81 | 0.453 | 0.238 |
| 82 | 0.272 | 0.256 |
| 83 | 0.035 | 0.020 |
| 84 | 0.016 | 0.295 |
| 85 | 0.018 | 0.023 |
| 86 | 3.000 | 0.580 |
| 87 | 2.000 | 0.480 |
| 88 | 0.015 | 0.065 |
| 89 | 0.645 | 0.544 |
| 90 | 0.195 | 0.815 |
| 91 | 0.030 | 0.021 |
| 92 | 0.027 | 0.262 |
| 93 | 0.102 | 0.225 |
| 94 | 0.380 | 0.630 |
| 95 | 2.800 | 10.000 |
| 96 | 0.200 | 1.100 |
| 97 | 0.270 | 5.000 |
| 98 | 0.022 | 0.300 |
| 99 | 0.016 | 0.260 |
| 100 | >10 | >10 |
| 101 | 6.299 | 10.000 |
| 102 | >10 | >10 |
| 103 | 0.120 | 0.110 |
| 104 | 0.037 | 0.690 |
| 105 | 0.100 | 0.013 |
| 106 | 0.080 | 0.027 |
| 107 | 0.080 | 0.037 |
| 108 | 0.140 | 0.050 |
| 109 | 0.006 | 0.004 |
| 110 | 0.045 | 0.042 |
| 111 | 0.035 | 0.040 |
| 112 | 0.040 | 0.270 |
| 113 | 0.020 | 0.030 |
| 114 | 0.020 | 0.020 |
| 115 | 0.040 | 0.025 |
| 116 | 0.400 | 0.760 |
| 117 | 0.130 | 0.440 |
| 118 | 1.800 | 8.000 |
| 119 | 0.045 | 0.009 |
| 120 | 0.080 | 0.120 |
| 121 | 0.017 | 0.025 |
| 122 | 0.148 | 0.115 |
| 123 | 0.020 | 0.040 |
| 124 | 0.055 | 0.200 |
| 125 | 0.015 | 0.012 |
| 126 | 0.060 | 0.200 |
| 127 | 0.040 | 0.040 |
| 128 | 0.230 | 0.400 |
| 129 | 0.080 | 0.120 |
| 130 | 0.020 | 0.004 |
| 131 | 0.007 | 0.004 |
| 132 | 0.023 | 0.005 |
| 133 | 0.024 | 0.115 |
| 134 | 0.010 | 0.100 |
| 135 | 0.825 | 10.000 |
| 136 | 0.055 | 1.000 |
| 137 | 0.060 | 0.360 |
| 138 | 5.000 | >10 |
| 139 | 0.860 | >10 |
| 140 | 0.700 | 6.001 |
| 141 | 0.050 | 0.870 |
| 142 | 2.000 | 10.000 |
| 143 | >10 | >10 |
| 144 | 0.350 | 3.000 |
| 145 | 0.016 | 0.430 |
| 146 | 3.000 | 10.000 |
| 147 | 10.000 | >10 |
| 148 | 5.000 | >10 |
| 149 | 10.000 | 10.000 |
| 150 | 0.400 | 1.200 |
| 151 | 3.000 | 6.001 |
| 152 | 0.330 | 5.000 |
| 153 | 0.460 | 8.000 |
| 154 | 1.300 | 10.000 |
| 155 | 0.020 | 0.270 |
| 156 | 0.043 | 5.000 |
| 157 | 0.080 | 0.360 |
| 158 | 0.010 | 0.150 |
| 159 | 0.050 | 0.300 |
| 160 | 3.000 | 8.000 |
| 161 | 0.004 | 0.160 |
| 162 | 0.240 | 6.400 |
| 163 | 1.000 | >10 |
| 164 | 0.141 | 7.000 |
| 165 | 8.000 | 6.001 |
| 166 | 0.013 | 0.007 |
| 167 | 0.340 | 0.080 |
| 168 | 1.200 | 0.480 |
| 169 | 0.610 | 0.600 |
| 170 | 1.200 | 1.800 |
| 171 | 0.200 | 0.035 |
| 172 | 0.050 | 0.010 |
| 173 | 0.225 | 0.200 |
| 174 | 0.100 | 0.030 |
| 175 | 10.000 | 1.000 |
| 176 | 2.000 | 0.450 |
| 177 | 5.000 | 1.700 |
| 178 | >10 | >10 |
| 179 | 1.600 | 1.200 |
| 180 | >10 | 10.000 |
| 181 | 5.000 | 1.600 |
| 182 | 1.700 | 0.340 |
| 183 | 1.400 | 0.430 |
| 184 | 10.000 | 10.000 |
| 185 | 0.450 | 0.440 |
| 186 | 0.330 | 0.160 |
| 187 | 1.600 | 1.000 |
| 188 | 0.080 | 0.020 |
| 189 | 0.070 | 0.017 |
| 190 | 3.000 | 3.700 |
| 191 | 3.000 | >10 |
| 192 | 4.000 | >10 |
| 193 | 0.327 | 6.001 |
| 194 | 0.220 | 4.000 |
| 195 | 0.110 | 0.310 |
| 196 | 6.001 | 1.600 |
| 197 | 1.300 | 8.999 |
| 198 | 0.390 | 6.001 |
| 199 | 0.280 | 0.590 |
| 200 | 0.038 | 0.500 |
| 201 | >10 | >10 |
| 202 | 0.045 | 0.085 |
| 203 | 0.210 | 0.350 |
| 204 | 0.014 | 0.032 |
| 205 | 0.106 | 0.225 |
| 206 | 0.011 | 0.021 |
| 207 | 0.034 | 0.046 |
| 208 | 0.021 | 0.017 |
| 209 | 0.009 | 0.002 |
| 210 | 1.750 | 1.160 |
| 211 | 3.000 | >10 |
| 212 | 0.500 | 8.000 |
| 213 | 0.100 | 0.320 |
| 214 | 0.015 | 0.090 |
| 215 | 0.001 | 0.030 |
| 216 | >10 | >10 |
| 217 | 0.150 | 0.250 |
| 218 | 0.170 | 0.700 |
| 219 | 0.230 | 10.000 |
| 220 | 0.320 | 0.430 |
| 221 | 0.003 | 0.050 |
| 222 | 0.700 | 0.600 |
| 223 | 0.180 | 10.000 |
| 224 | 4.000 | >10 |
| 225 | 0.043 | 0.535 |
| 226 | 0.006 | 0.040 |
| 227 | 0.400 | 5.300 |
| 228 | 0.030 | 0.080 |
| 229 | 0.200 | 3.000 |
| 230 | 4.000 | 6.001 |
| 231 | 0.130 | 3.600 |
| 232 | 0.040 | 0.240 |
| 233 | 0.010 | 0.140 |
| 234 | 0.006 | 0.012 |
| 235 | 0.600 | 6.001 |
| 236 | 0.300 | 8.000 |
| 237 | 0.005 | 0.030 |
| 238 | 0.200 | 1.600 |
| 239 | 0.700 | 6.001 |
| 240 | 0.020 | 0.080 |
| 241 | 0.008 | 0.370 |
| 242 | 0.009 | 0.050 |
| 243 | 0.002 | 0.009 |
| 244 | 1.400 | 7.000 |
| 245 | 0.019 | 0.015 |
| 246 | 0.010 | 0.199 |

### Assay Method 3—Rat Mild Thermal Injury Model (MTI)

Pathogen-free, male albino Sprague-Dawley rats were purchased from Harlan Industries (San Diego, Calif.) and maintained on a 12-h light/dark cycle (lights on at 9:00 AM and off at 9:00 PM) in a climate-controlled room. Food and water were available ad libitum up to the time of the testing.

Under isoflurane/oxygen anesthesia, a first-degree burn injury (erythema without blistering) was produced as follows: the plantar surface of the rat's left hind paw was placed on water-dampened 56° C. hotplate for 20 seconds and steady contact was maintained by applying an 84 g weight to the dorsum (after Nozaki-Taguchi & Yaksh, Neurosci. Lett. 1998, 254, 25-28).

Mild thermal injury results in mechanical allodynia in the left hind paw. Mechanical (tactile) allodynia was assessed by determining the median threshold at which the affected paw was withdrawn from graded stimuli (von Frey filaments ranging from 0.41 to 15.8 g) applied perpendicularly with sufficient force to bend slightly and held for 2-3 seconds against the proximal half of the third and fourth toe surfaces through wire-mesh observation cages. Paw flinching during or immediately following the removal of the stimulus was considered a positive response. A paw withdrawal threshold (PWT) was determined by sequentially increasing and decreasing the stimulus strength and analyzing withdrawal data using an adaptation of the Dixon up-down method, as described (Chaplan et al., J. Neurosci. 1994, 53, 55-63). Rats were included in the study only if their baseline PWT was 3.1623 g or lower (4.5 logarithmically).

Rats were tested for pre-injury thresholds prior to mild thermal injury and again for baseline thresholds after development of mechanical allodynia. Immediately after baseline measurement, test compound or vehicle was administered orally and the measurement was repeated at 0.5 h after administration. The tactile thresholds (log value) were converted to percent of a maximum possible effect ( %MPE): % MPE=[Threshold(t)−Threshold(baseline)]*100/[Threshold(pre)−Threshold(baseline)], where t=post-treatment time. Data were expressed as mean±standard error of the mean (S.E.M.). Statistical analysis was performed using two-way ANOVA with repeated measures with a significance level of p<0.05.

Results for compounds tested in this assay are presented in Table 2, as an average of results obtained. Compounds were tested in free base, hydrochloride salt, and/or trifluoroacetic acid salt forms.

TABLE 2

| Ex. | Dose p.o. | % MPE |
|---|---|---|
| 36 | 20 mg/Kg | 38 +/− 13 |
| 46 | 6 mg/Kg | 0 |
| 58 | 20 mg/Kg | 52 +/− 7 |
| 109 | 10 mg/Kg | 0 |
| 245 | 10 mg/Kg | 36 +/− 10 |
| 246 | 20 mg/Kg | 32 +/− 3 |

While the invention has been illustrated by reference to exemplary and preferred embodiments, it will be understood that the invention is intended not to be limited to the foregoing detailed description, but to be defined by the appended claims as properly construed under principles of patent law.

What is claimed is:

1. A compound of Formula (I): 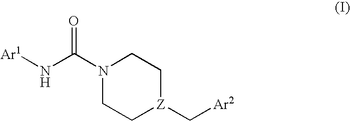
wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, 1H-pyrazol-3-yl, 5-methylpyrazin-2-yl, 3-chloropyrazin-2-yl, pyridazin-3-yl, 6-methoxypyridazin-3-yl, 5-methylisoxazol-3-yl, 1,5-dimethyl-1H-pyrazol-3-yl, 4-bromo-1-methyl-1H-pyrazol-3-yl, 2-ethyl-2H-pyrazol-3-yl, 5-methyl-1H-pyrazol-3-yl, or 5-phenyl-1H-pyrazol-3-yl group;
Z is —N— or >CH; and
Ar² is:
(i) phenyl unsubstituted or substituted with one or two R^{a} moieties;
where each R^{a} moiety is independently —C₁₋₄alkyl, —C≡C—R^{d}, —OC₁₋₄alkyl, halo, —CF₃, —OCF₃, —OCH₂CF₃, —SCF₃, —S(O)₀₋₂C₁₋₄alkyl, —SO₂CF₃, —OSO₂C₁₋₄alkyl, —(CH₂)₀₋₁CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —(CH₂)₀₋₁CN;
or two adjacent R^{a} moieties taken together form —O(CH₂)₁₋₂O— or —OCF₂O—;
where R^{b} and R^{c} are each independently —H or —C₁₋₄alkyl; and
R^{d} is H, C₃₋₆cycloalkyl, or —CH₂NR^{e}R^{f};
where R^{e} and R^{f} are each independently H or C₁₋₄alkyl;
(ii) phenyl substituted at the 3- or 4-position with -L-Ar³, unsubstituted or substituted with R^{a}, wherein:
L is a linker selected from the group consisting of —(CH₂)₁₋₃—, —CH═CH—, —O—, —OCH₂—, —CH₂O—, —NH—, >NC₁₋₄alkyl, —S—, —C≡C—, —C(═O)—, and a covalent bond; and
Ar³ is:
(a) phenyl;
(b) naphthyl; or
(c) a monocyclic or bicyclic heteroaryl group; or
(iii) a 9- or 10-membered fused bicyclic heteroaryl group;
where when Ar¹ is 6-chloro-pyridazin-3-yl, isoxazol-3-yl, or 1H-pyrazol-3-yl,
then Ar² is not benzo[1,3]dioxol-5-yl or 2,2-difluoro-benzo[1,3]dioxol-5-yl;
or a pharmaceutically acceptable salt, pharmaceutically acceptable prodrug, or pharmaceutically active metabolite of said compound.

2. A compound or pharmaceutically acceptable salt as defined in claim 1, wherein Ar¹ is a benzo[d]isoxazol-3-yl, 6-fluorobenzo[d]isoxazol-3-yl, benzo[1,2,5]thiadiazol-4-yl, benzo[1,2,5]oxadiazol-4-yl, 6-chloro-pyridazin-3-yl, pyrazin-2-yl, isoxazol-3-yl, 1H-benzotriazol-5-yl, benzothiazol-6-yl, or 1H-pyrazol-3-yl group.

3. A compound or pharmaceutically acceptable salt as defined in claim 1, wherein Ar¹ is a benzo[d]isoxazol-3-yl group.

4. A compound or pharmaceutically acceptable salt as defined in claim 1, wherein Ar¹ is a pyrazin-2-yl group.

5. A compound or pharmaceutically acceptable salt as defined in claim 1, wherein Ar¹ is an isoxazol-3-yl group.

6. A compound or pharmaceutically acceptable salt as defined in claim 1, wherein Ar¹ is a pyridazin-3-yl group.

7. A compound or pharmaceutically acceptable salt as defined in claim 1, wherein Z is —N—.

8. A compound or pharmaceutically acceptable salt as defined in claim 1, wherein Z is >CH.

9. A compound or pharmaceutically acceptable salt as defined in claim 1, wherein Ar² is phenyl, substituted with one or two R^{a} moieties.

10. A compound or pharmaceutically acceptable salt as defined in claim 9, wherein each R^{a} moiety is independently selected from the group consisting of: chloro, cyano, isobutyl, methylsulfanyl, methanesulfonyl, trifluoromethyl, trifluoromethoxy, 2,2,2-trifluoroethoxy, fluoro, methyl, methoxy, tert-butyl, bromo, methoxycarbonyl, cyanomethyl, methoxycarbonylmethyl, trifluoromethanesulfonyl, trifluoromethanesulfanyl, and butyl; or two adjacent R^{a} moieties taken together form —OCH₂O— or —OCF₂O—.

11. A compound or pharmaceutically acceptable salt as defined in claim 1, wherein Ar² is phenyl substituted at the 3- or 4-position with -L-Ar³, unsubstituted or substituted with one or two R^{a} moieties.

12. A compound or pharmaceutically acceptable salt as defined in claim 11, wherein L is —CH₂CH₂—, —O—, —OCH₂—, or —C≡C—.

13. A compound or pharmaceutically acceptable salt as defined in claim 11, wherein Ar³ is phenyl.

14. A compound or pharmaceutically acceptable salt as defined in claim 13, wherein each R^{a} moiety is independently selected from the group consisting of: chloro, cyano, isobutyl, methylsulfanyl, methanesulfonyl, trifluoromethyl, trifluoromethoxy, 2,2,2-trifluoroethoxy, fluoro, methyl, methoxy, tert-butyl, bromo, methoxycarbonyl, cyanomethyl, methoxycarbonylmethyl, trifluoromethanesulfonyl, trifluoromethanesulfanyl, and butyl; or two adjacent R^{a} moieties taken together form —OCH₂O— or —OCF₂O—.

15. A compound or pharmaceutically acceptable salt as defined in claim 11, wherein Ar³ is naphthyl.

16. A compound or pharmaceutically acceptable salt as defined in claim 11, wherein Ar³ is a monocyclic or bicyclic heteroaryl group.

17. A compound or pharmaceutically acceptable salt as defined in claim 16, wherein Ar³ is a thiophenyl, pyrimidinyl, pyridyl, pyrazinyl, or quinolinyl group.

18. A compound or pharmaceutically acceptable salt as defined in claim 1, wherein Ar² is a 9- or 10-membered fused bicyclic heteroaryl group.

19. A compound or pharmaceutically acceptable salt as defined in claim 18, wherein Ar² is a benzimidazolyl, indazolyl, benzothiophenyl, quinolinyl, indolyl, or benzofuranyl group.

20. A compound of Formula (Ia): 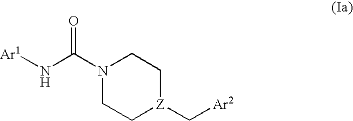
wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, or 1H-pyrazol-3-yl group;
Z is —N— or >CH; and
Ar² is:
(i) phenyl or 3-phenoxyphenyl substituted with one or two R^{a} moieties;
where each R^{a} moiety is independently —C₁₋₄alkyl, —OC₁₋₄alkyl, halo, —CF₃, —OCF₃, —OCH₂CF₃, —SCF₃, —S(O)₀₋₂C₁₋₄alkyl, -OSO₂C₁₄alkyl, —CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —CN;
where R^{b} and R^{c} are each independently —H or —C₁₋₄alkyl; or
(ii) benzo[1,3]dioxol-5-yl, 2,2-difluoro-benzo[1,3]dioxol-5-yl, or naphthyl;
where when Ar¹ is 6-chloro-pyridazin-3-yl, isoxazol-3-yl, or 1H-pyrazol-3-yl,
then Ar² is not benzo[1,3]dioxol-5-yl or 2,2-difluoro-benzo[1,3]dioxol-5-yl;
or a pharmaceutically acceptable salt, pharmaceutically acceptable prodrug, or pharmaceutically active metabolite of said compound.

21. A compound or pharmaceutically acceptable salt as defined in claim 20, wherein Ar¹ is a benzo[d]isoxazol-3-yl, 6-fluorobenzo[d]isoxazol-3-yl, benzo[1,2,5]thiadiazol-4-yl, benzo[1,2,5]oxadiazol-4-yl, 6-chloro-pyridazin-3-yl, pyrazin-2-yl, isoxazol-3-yl, 1H-benzotriazol-5-yl, benzothiazol-6-yl, or 1H-pyrazol-3-yl group.

22. A compound or pharmaceutically acceptable salt as defined in claim 20, wherein Ar¹ is a benzo[d]isoxazol-3-yl group.

23. A compound or pharmaceutically acceptable salt as defined in claim 20, wherein Ar¹ is a pyrazin-2-yl group.

24. A compound or pharmaceutically acceptable salt as defined in claim 20, wherein Ar¹ is an isoxazol-3-yl group.

25. A compound or pharmaceutically acceptable salt as defined in claim 20, wherein Ar¹ is a pyridazin-3-yl group.

26. A compound or pharmaceutically acceptable salt as defined in claim 20, wherein Ar² is 3-phenoxyphenyl substituted with one or two R^{a} moieties independently selected from the group consisting of fluoro, chloro, bromo, —CF₃, —OCF₃, and —OCH₂CF₃.

27. A compound or pharmaceutically acceptable salt as defined in claim 20, wherein Ar² is naphthyl.

28. A compound or pharmaceutically acceptable salt selected from the group consisting of:
4-(2,2-Difluoro-benzo[1,3]dioxol-5-ylmethyl)-piperazine-1-carboxylic acid benzo[d]isoxazol-3-ylamide;
4-(2,2-Difluoro-benzo[1,3]dioxol-5-ylmethyl)-piperazine-1-carboxylic acid (3-phenyl-[1,2,4]thiadiazol-5-yl)-amide;
4-(2,2-Difluoro-benzo[1,3]dioxol-5-ylmethyl)-piperazine-1-carboxylic acid (1H-tetrazol-5-yl)-amide;
4-(2,2-Difluoro-benzo[1,3]dioxol-5-ylmethyl)-piperazine-1-carboxylic acid benzo[1,2,5]thiadiazol-4-ylamide;
4-(2,2-Difluoro-benzo[1,3]dioxol-5-ylmethyl)-piperazine-1-carboxylic acid benzo[1,2,5]oxadiazol-4-ylamide;
4-(2,2-Difluoro-benzo[1,3]dioxol-5-ylmethyl)-piperazine-1-carboxylic acid (3H-benzotriazol-5-yl)-amide;
4-(2,2-Difluoro-benzo[1,3]dioxol-5-ylmethyl)-piperazine-1-carboxylic acid thiophen-2-ylamide;
4-(2,2-Difluoro-benzo[1,3]dioxol-5-ylmethyl)-piperazine-1-carboxylic acid thiophen-3-ylamide;
4-Naphthalen-2-ylmethyl-piperidine-1-carboxylic acid (6-chloro-pyridazin-3-yl)-amide;
4-Naphthalen-2-ylmethyl-piperidine-1-carboxylic acid pyrazin-2-ylamide;
4-Naphthalen-2-ylmethyl-piperidine-1-carboxylic acid isoxazol-3-ylamide;
4-Naphthalen-2-ylmethyl-piperidine-1-carboxylic acid (3-phenyl-[1,2,4]thiadiazol-5-yl)-amide;
4-Naphthalen-2-ylmethyl-piperidine-1-carboxylic acid (1H-tetrazol-5-yl)-amide;
4-Naphthalen-2-ylmethyl-piperidine-1-carboxylic acid (2H-pyrazol-3-yl)-amide;
4-Naphthalen-2-ylmethyl-piperidine-1-carboxylic acid benzo[1,2,5]oxadiazol-4-ylamide;
4-Naphthalen-2-ylmethyl-piperidine-1-carboxylic acid (1H-benzotriazol-5-yl)-amide;
4-Naphthalen-2-ylmethyl-piperidine-1-carboxylic acid [1,5]naphthyridin-2-ylamide;
4-Naphthalen-2-ylmethyl-piperidine-1-carboxylic acid quinolin-2-ylamide;
4-Naphthalen-2-ylmethyl-piperidine-1-carboxylic acid benzothiazol-6-ylamide;
4-Naphthalen-2-ylmethyl-piperidine-1-carboxylic acid quinolin-5-ylamide;
4-Naphthalen-2-ylmethyl-piperidine-1-carboxylic acid benzo[d]isoxazol-3-ylamide;
4-(4-Fluoro-benzyl)-piperidine-1-carboxylic acid benzo[d]isoxazol-3-ylamide;
4-(4-Fluoro-benzyl)-piperidine-1-carboxylic acid (6-chloro-pyridazin-3-yl)-amide;
4-(4-Fluoro-benzyl)-piperidine-1-carboxylic acid isoxazol-3-ylamide;
4-(3-Trifluoromethyl-benzyl)-piperidine-1-carboxylic acid (6-chloro-pyridazin-3-yl)-amide;
4-(3-Trifluoromethyl-benzyl)-piperidine-1-carboxylic acid isoxazol-3-ylamide;
4-(3-Trifluoromethyl-benzyl)-piperidine-1-carboxylic acid benzo[d]isoxazol-3-ylamide;
4-[3-(4-Fluoro-3-trifluoromethyl-phenoxy)-benzyl]-piperazine-1-carboxylic acid benzo[d]isoxazol-3-ylamide;
4-[3-(3-Trifluoromethoxy-phenoxy)-benzyl]-piperazine-1-carboxylic acid benzo[d]isoxazol-3-ylamide;
4-[3-(4-Trifluoromethoxy-phenoxy)-benzyl]-piperazine-1-carboxylic acid benzo[d]isoxazol-3-ylamide;
4-[3-(3-Bromo-phenoxy)-benzyl]-piperazine-1-carboxylic acid benzo[d]isoxazol-3-ylamide;
4-[3-(4-Bromo-phenoxy)-benzyl]-piperazine-1-carboxylic acid benzo[d]isoxazol-3-ylamide;
4-[3-(3,4-Difluoro-phenoxy)-benzyl]-piperazine-1-carboxylic acid benzo[d]isoxazol-3-ylamide;
4-[3-(3,5-Difluoro-phenoxy)-benzyl]-piperazine-1-carboxylic acid benzo[d]isoxazol-3-ylamide;
4-{3-[4-(2,2,2-Trifluoro-ethoxy)-phenoxy]-benzyl}-piperazine-1-carboxylic acid benzo[d]isoxazol-3-ylamide;
4-[3-(4-Chloro-phenoxy)-benzyl]-piperazine-1-carboxylic acid benzo[d]isoxazol-3-ylamide;
4-[3-(4-Chloro-phenoxy)-benzyl]-piperazine-1-carboxylic acid benzo[1,2,5]thiadiazol-4-ylamide;
4-[3-(3,4-Dichloro-phenoxy)-benzyl]-piperazine-1-carboxylic acid benzo[1,2,5]thiadiazol-4-ylamide;
4-[3-(3,5-Dichloro-phenoxy)-benzyl]-piperazine-1-carboxylic acid benzo[1,2,5]thiadiazol-4-ylamide;
4-[3-(3-Trifluoromethyl-phenoxy)-benzyl]-piperazine-1-carboxylic acid benzo[1,2,5]thiadiazol-4-ylamide;
4-(3-Trifluoromethoxy-benzyl)-piperazine-1-carboxylic acid benzo[1,2,5]thiadiazol-4-ylamide;
4-[3-(4-Chloro-phenoxy)-benzyl]-piperazine-1-carboxylic acid (6-chloro-pyridazin-3-yl)-amide;
4-[3-(3,4-Dichloro-phenoxy)-benzyl]-piperazine-1-carboxylic acid (6-chloro-pyridazin-3-yl)-amide;
4-[3-(3-Trifluoromethyl-phenoxy)-benzyl]-piperazine-1-carboxylic acid (6-chloro-pyridazin-3-yl)-amide;
4-(4-Fluoro-3-phenoxy-benzyl)-piperazine-1-carboxylic acid (6-chloro-pyridazin-3-yl)-amide;
4-[3-(4-Chloro-phenoxy)-benzyl]-piperazine-1-carboxylic acid isoxazol-3-ylamide;
4-[3-(3,4-Dichloro-phenoxy)-benzyl]-piperazine-1-carboxylic acid isoxazol-3-ylamide;
4-[3-(3-Trifluoromethyl-phenoxy)-benzyl]-piperazine-1-carboxylic acid isoxazol-3-ylamide;
4-(4-Fluoro-3-phenoxy-benzyl)-piperazine-1-carboxylic acid isoxazol-3-ylamide;
4-[3-(3,4-Dichloro-phenoxy)-benzyl]-piperazine-1-carboxylic acid benzo[d]isoxazol-3-ylamide;
4-[3-(3-Trifluoromethyl-phenoxy)-benzyl]-piperazine-1-carboxylic acid benzo[d]isoxazol-3-ylamide;
4-(3-Trifluoromethoxy-benzyl)-piperazine-1-carboxylic acid benzo[d]isoxazol-3-ylamide;
4-[3-(4-Chloro-phenoxy)-benzyl]-piperazine-1-carboxylic acid (1H-pyrazol-3-yl)-amide;
4-[3-(3,4-Dichloro-phenoxy)-benzyl]-piperazine-1-carboxylic acid (1H-pyrazol-3-yl)-amide;
4-[3-(3-Trifluoromethyl-phenoxy)-benzyl]-piperazine-1-carboxylic acid (1H-pyrazol-3-yl)-amide;
4-(4-Fluoro-3-phenoxy-benzyl)-piperazine-1-carboxylic acid (1H-pyrazol-3-yl)-amide;
4-[3-(4-Chloro-phenoxy)-benzyl]-piperazine-1-carboxylic acid (1H-tetrazol-5-yl)-amide;
4-[3-(4-Chloro-phenoxy)-benzyl]-piperazine-1-carboxylic acid pyrazin-2-ylamide;
4-[3-(3,4-Dichloro-phenoxy)-benzyl]-piperazine-1-carboxylic acid pyrazin-2-ylamide;
4-[3-(3-Trifluoromethyl-phenoxy)-benzyl]-piperazine-1-carboxylic acid pyrazin-2-ylamide; and
4-(3-Trifluoromethoxy-benzyl)-piperazine-1-carboxylic acid pyrazin-2-ylamide;
and pharmaceutically acceptable salts thereof.

29. A compound or pharmaceutically acceptable salt selected from the group consisting of:
N-1,2-Benzisoxazol-3-yl-4-[(2,2-difluoro-1,3-benzodioxol-5-yl)methyl]piperidine-1-carboxamide;
4-(3-o-Tolylethynyl-benzyl)-piperazine-1-carboxylic acid benzo[d]isoxazol-3-ylamide;
N-1,2-Benzisoxazol-3-yl-4-(3-{[2-(trifluoromethyl)-phenyl]-ethynyl}benzyl)-piperazine-1-carboxamide;
N-1,2-Benzisoxazol-3-yl-4-{3-[(2-methoxyphenyl)-ethynyl]-benzyl}-piperazine-1-carboxamide;
N-1,2-Benzisoxazol-3-yl-4-{3-[(2-fluorophenyl)ethynyl]-benzyl}-piperazine-1-carboxamide;
N-1,2-Benzisoxazol-3-yl-4-{3-[(2-bromophenyl)-ethynyl]benzyl}-piperazine-1-carboxamide;
4-(3-Ethynyl-benzyl)-piperazine-1-carboxylic acid benzo[d]isoxazol-3-ylamide;
N-1,2-Benzisoxazol-3-yl-4-{3-[3-(dimethylamino)prop-1-yn-1-yl]benzyl}-piperazine-1-carboxamide;
N-1,2-Benzisoxazol-3-yl-4-[3-(cyclohexylethynyl)benzyl]-piperazine-1-carboxamide;
N-1,2-Benzisoxazol-3-yl-4-[3-(cyclopentylethynyl)benzyl]-piperazine-1-carboxamide;
N-1,2-Benzisoxazol-3-yl-4-{3-[(2-chlorophenyl)-ethynyl]benzyl}-piperazine-1-carboxamide;
N-1,2-Benzisoxazol-3-yl-4-{3-[(3-chlorophenyl)ethynyl]benzyl}-piperazine-1-carboxamide;
N-1,2-Benzisoxazol-3-yl-4-{3-[(4-chlorophenyl)ethynyl]benzyl}-piperazine-1-carboxamide;
N-1,2-Benzisoxazol-3-yl-4-{3-[(3,4-dichlorophenyl)ethynyl]-benzyl}-piperazine-1-carboxamide;
N-1,2-Benzisoxazol-3-yl-4-[3-(cyclopropylethynyl)benzyl]-piperazine-1-carboxamide;
N-1,2-Benzisoxazol-3-yl-4-[3-(thiophen-3-ylethynyl)benzyl]-piperazine-1-carboxamide;
4-{3-[(2-Chlorophenyl)ethynyl]benzyl}-N-pyrazin-2-ylpiperazine-1-carboxamide;
4-{3-[(2-Chlorophenyl)ethynyl]benzyl}-N-pyridazin-3-ylpiperazine-1-carboxamide;
4-{3-[(2-Chlorophenyl)ethynyl]benzyl}-N-(5-methylpyrazin-2-yl)piperazine-1-carboxamide;
N-1,2-Benzisoxazol-3-yl-4-{3-[(2,4-dichlorophenyl)-ethynyl]benzyl}-piperazine-1-carboxamide;
N-1,2-Benzisoxazol-3-yl-4-(3-{[2-(trifluoromethoxy)phenyl]-ethynyl}benzyl)-piperazine-1-carboxamide;
N-1,2-Benzisoxazol-3-yl-4-{3-[(3,5-dichlorophenyl)ethynyl]benzyl}-piperazine-1-carboxamide;
N-1,2-Benzisoxazol-3-yl-4-{3-[(2,5-dichlorophenyl)ethynyl]benzyl}-piperazine-1-carboxamide;
N-1,2-Benzisoxazol-3-yl-4-{3-[(2-cyanophenyl)ethynyl]benzyl}-piperazine-1-carboxamide;
N-1,2-Benzisoxazol-3-yl-4-[3-(naphthalen-1-ylethynyl)benzyl]piperazine-1-carboxamide;
Methyl 2-[(3-{[4-(1,2-benzisoxazol-3-ylcarbamoyl)piperazin-1-yl]methyl}phenyl)ethynyl]benzoate;
N-1,2-Benzisoxazol-3-yl-4-{3-[(3-cyanophenyl)ethynyl]benzyl}piperazine-1-carboxamide;
N-1,2-Benzisoxazol-3-yl-4-[3-(1,3-benzodioxol-5-ylethynyl)benzyl]-piperazine-1-carboxamide;
N-1,2-Benzisoxazol-3-yl-4-{3-[(2,3-dichlorophenyl)ethynyl]benzyl}-piperazine-1-carboxamide;
N-1,2-Benzisoxazol-3-yl-4-{3-[(2-cyano-3-fluorophenyl)ethynyl]-benzyl}piperazine-1-carboxamide;
N-1,2-Benzisoxazol-3-yl-4-(3-{[2-(cyanomethyl)phenyl]ethynyl}-benzyl)piperazine-1-carboxamide;
Methyl{2-[(3-{[4-(1,2-benzisoxazol-3-ylcarbamoyl)piperazin-1-yl]methyl}phenyl)ethynyl]phenyl}acetate;
4-[3-(2-o-Tolyl-ethyl)-benzyl]-piperazine-1-carboxylic acid benzo[d]isoxazol-3-ylamide;
4-[3-(Pyrimidin-2-yloxy)-benzyl]-piperazine-1-carboxylic acid benzo[d]isoxazol-3-ylamide;
N-1,2-Benzisoxazol-3-yl-4-[3-(pyridin-2-yloxy)benzyl]piperazine-1-carboxamide;
N-1,2-Benzisoxazol-3-yl-4-[3-(pyrazin-2-yloxy)benzyl]piperazine-1-carboxamide;
4-[3-(2-Cyano-benzyloxy)-benzyl]-piperazine-1-carboxylic acid benzo[d]isoxazol-3-ylamide;
N-1,2-Benzisoxazol-3-yl-4-[3-(benzyloxy)benzyl]piperazine-1-carboxamide;
4-(1H-Benzimidazol-6-ylmethyl)-N-1,2-benzisoxazol-3-ylpiperazine-1-carboxamide;
N-1,2-Benzisoxazol-3-yl-4-(1H-indazol-6-ylmethyl)piperazine-1-carboxamide;
N-1,2-Benzisoxazol-3-yl-4-[4-(methylsulfonyl)benzyl]piperazine-1-carboxamide;
N-1,2-Benzisoxazol-3-yl-4-[4-(trifluoromethoxy)benzyl]piperazine-1-carboxamide;
4-[3-(4-Chlorophenoxy)benzyl]-N-(6-methoxypyridazin-3-yl)piperazine-1-carboxamide;
N-1,2-Benzisoxazol-3-yl-4-[4-chloro-3-(trifluoromethoxy)benzyl]-piperazine-1-carboxamide;
N-1,2-Benzisoxazol-3-yl-4-[4-fluoro-3-(trifluoromethoxy)benzyl]-piperazine-1-carboxamide;
N-1,2-Benzisoxazol-3-yl-4-[3-chloro-4-(trifluoromethoxy)benzyl]-piperazine-1-carboxamide;
N-1,2-Benzisoxazol-3-yl-4-[3-fluoro-4-(trifluoromethoxy)benzyl]-piperazine-1-carboxamide;
N-1,2-Benzisoxazol-3-yl-4-{3-[4-(trifluoromethyl)phenoxy]benzyl}-piperazine-1-carboxamide;
N-1,2-Benzisoxazol-3-yl-4-(3-phenoxybenzyl)piperazine-1-carboxamide;
N-1,2-Benzisoxazol-3-yl-4-(3,4-dichlorobenzyl)piperazine-1-carboxamide;
N-1,2-Benzisoxazol-3-yl-4-[4-(benzyloxy)benzyl]piperazine-1-carboxamide;
N-1,2-Benzisoxazol-3-yl-4-(1-benzothiophen-2-ylmethyl)piperazine-1-carboxamide;
N-1,2-Benzisoxazol-3-yl-4-[3-(quinolin-6-yloxy)benzyl]piperazine-1-carboxamide;
N-1,2-Benzisoxazol-3-yl-4-(4-bromo-3-fluorobenzyl)piperazine-1-carboxamide;
N-1,2-Benzisoxazol-3-yl-4-(1,3-benzodioxol-5-ylmethyl)piperazine-1-carboxamide;
N-1,2-Benzisoxazol-3-yl-4-(quinolin-3-ylmethyl)piperazine-1-carboxamide;
N-1,2-Benzisoxazol-3-yl-4-(1H-indol-5-ylmethyl)piperazine-1-carboxamide;
N-1,2-Benzisoxazol-3-yl-4-[3-(naphthalen-2-yloxy)benzyl]piperazine-1-carboxamide;
N-1,2-Benzisoxazol-3-yl-4-(4-bromobenzyl)piperazine-1-carboxamide;
N-1,2-Benzisoxazol-3-yl-4-(3,4-dibromobenzyl)piperazine-1-carboxamide;
N-1,2-Benzisoxazol-3-yl-4-[3-(2-chlorophenoxy)benzyl]piperazine-1-carboxamide;
4-Naphthalen-2-ylmethyl-piperazine-1-carboxylic acid benzo[d]isoxazol-3-ylamide;
4-Quinolin-2-ylmethyl-piperazine-1-carboxylic acid benzo[d]isoxazol-3-ylamide;
4-[3-(4-Cyano-phenoxy)-benzyl]-piperazine-1-carboxylic acid benzo[d]isoxazol-3-ylamide;
4-Benzofuran-2-ylmethyl-piperazine-1-carboxylic acid benzo[d]isoxazol-3-ylamide;
N-1,2-Benzisoxazol-3-yl-4-[3-(3-chlorophenoxy)benzyl]piperazine-1-carboxamide;
N-1,2-Benzisoxazol-3-yl-4-{3-[4-cyano-3-(trifluoromethyl)phenoxy]benzyl}piperazine-1-carboxamide;
N-1,2-Benzisoxazol-3-yl-4-[3-(3-cyanophenoxy)benzyl]piperazine-1-carboxamide;
N-1,2-Benzisoxazol-3-yl-4-(3-{4-[(trifluoromethyl)sulfanyl]phenoxy}-benzyl)piperazine-1-carboxamide;
N-1,2-Benzisoxazol-3-yl-4-{3-[(2,2-difluoro-1,3-benzodioxol-5-yl)oxy]benzyl}piperazine-1-carboxamide;
N-1,2-Benzisoxazol-3-yl-4-(3-{4-[(trifluoromethyl)sulfonyl]phenoxy}-benzyl)piperazine-1-carboxamide;
N-1,2-Benzisoxazol-3-yl-4-{[3-(phenylethynyl)phenyl]methyl}piperazine-1-carboxamide;
N-Isoxazol-3-yl-4-{3-[4-(trifluoromethoxy)phenoxy]benzyl}piperazine-1-carboxamide;
4-[4-(Benzyloxy)benzyl]-N-isoxazol-3-ylpiperazine-1-carboxamide;
4-[3-(3-Chlorophenoxy)benzyl]-N-isoxazol-3-ylpiperazine-1-carboxamide;
N-isoxazol-3-yl-4-{3-[4-(2,2,2-trifluoroethoxy)phenoxy]benzyl}piperazine-1-carboxamide;
4-(1-Benzofuran-2-ylmethyl)-N-isoxazol-3-ylpiperazine-1-carboxamide;
4-[3-(3-Cyanophenoxy)benzyl]-N-isoxazol-3-ylpiperazine-1-carboxamide;
4-[3-(2-Chlorophenoxy)benzyl]-N-isoxazol-3-ylpiperazine-1-carboxamide;
4-{3-[(2,2-Difluoro-1,3-benzodioxol-5-yl)oxy]benzyl}-N-isoxazol-3-ylpiperazine-1-carboxamide;
4-(1-Benzothiophen-2-ylmethyl)-N-isoxazol-3-ylpiperazine-1-carboxamide;
4-(1,3-Benzodioxol-5-ylmethyl)-N-isoxazol-3-ylpiperazine-1-carboxamide;
N-Isoxazol-3-yl-4-(naphthalen-2-ylmethyl)piperazine-1-carboxamide;
4-[3-(4-Bromophenoxy)benzyl]-N-isoxazol-3-ylpiperazine-1-carboxamide;
4-Quinolin-2-ylmethyl-piperazine-1-carboxylic acid isoxazol-3-ylamide;
4-Quinolin-3-ylmethyl-piperazine-1-carboxylic acid isoxazol-3-ylamide;
4-(4-Bromo-benzyl)-piperazine-1-carboxylic acid isoxazol-3-ylamide;
4-(1H-Indol-6-ylmethyl)-piperazine-1-carboxylic acid isoxazol-3-ylamide;
4-[3-(Naphthalen-2-yloxy)-benzyl]-piperazine-1-carboxylic acid isoxazol-3-ylamide;
4-(4-Bromo-3-fluoro-benzyl)-piperazine-1-carboxylic acid isoxazol-3-ylamide;
4-[3-(4-Cyano-phenoxy)-benzyl]-piperazine-1-carboxylic acid isoxazol-3-ylamide;
4-[3-(3,4-Difluorophenoxy)benzyl]-N-isoxazol-3-ylpiperazine-1-carboxamide;
4-(3,4-Dibromobenzyl)-N-isoxazol-3-ylpiperazine-1-carboxamide;
N-Isoxazol-3-yl-4-(3-{4-[(trifluoromethyl)sulfanyl]phenoxy}benzyl)-piperazine-1-carboxamide;
4-{3-[4-Fluoro-3-(trifluoromethyl)phenoxy]benzyl}-N-isoxazol-3-ylpiperazine-1-carboxamide;
4-[3-(3-Bromophenoxy)benzyl]-N-isoxazol-3-ylpiperazine-1-carboxamide;
N-Isoxazol-3-yl-4-(3-{4-[(trifluoromethyl)sulfonyl]phenoxy}benzyl)-piperazine-1-carboxamide;
N-Isoxazol-3-yl-4-{3-[3-(trifluoromethoxy)phenoxy]benzyl}piperazine-1-carboxamide;
4-(3,4-Dichlorobenzyl)-N-isoxazol-3-ylpiperazine-1-carboxamide;
N-Isoxazol-3-yl-4-{3-[4-(trifluoromethyl)phenoxy]benzyl}piperazine-1-carboxamide;
N-Isoxazol-3-yl-4-[3-(quinolin-6-yloxy)benzyl]piperazine-1-carboxamide;
4-{3-[4-Cyano-3-(trifluoromethyl)phenoxy]benzyl}-N-isoxazol-3-ylpiperazine-1-carboxamide;
4-[3-(4-Chlorophenoxy)benzyl]-N-(5-methylisoxazol-3-yl)piperazine-1-carboxamide;
4-(Quinolin-3-ylmethyl)-N-1H-tetrazol-5-ylpiperazine-1-carboxamide;
4-[3-(Naphthalen-2-yloxy)benzyl]-N-1H-tetrazol-5-ylpiperazine-1-carboxamide;
4-(3,4-Dibromobenzyl)-N-1H-tetrazol-5-ylpiperazine-1-carboxamide;
4-(4-Bromo-3-fluorobenzyl)-N-1H-tetrazol-5-ylpiperazine-1-carboxamide;
4-[3-(3,4-Difluorophenoxy)benzyl]-N-1H-tetrazol-5-ylpiperazine-1-carboxamide;
4-{3-[4-Cyano-3-(trifluoromethyl)phenoxy]benzyl}-N-1H-tetrazol-5-ylpiperazine-1-carboxamide;
N-1H-Tetrazol-5-yl-4-{3-[4-(trifluoromethyl)phenoxy]benzyl}piperazine-1-carboxamide;
N-1H-Tetrazol-5-yl-4-(3-{4-[(trifluoromethyl)sulfanyl]phenoxy}benzyl)-piperazine-1-carboxamide;
N-1H-Tetrazol-5-yl-4-{3-[3-(trifluoromethoxy)phenoxy]benzyl}piperazine-1-carboxamide;
4-[3-(3,4-Dichlorophenoxy)benzyl]-N-1H-tetrazol-5-ylpiperazine-1-carboxamide;
4-(Quinolin-2-ylmethyl)-N-1H-tetrazol-5-ylpiperazine-1-carboxamide;
4-(Naphthalen-2-ylmethyl)-N-1H-tetrazol-5-ylpiperazine-1-carboxamide;
4-(4-Bromo-benzyl)-piperazine-1-carboxylic acid (2H-tetrazol-5-yl)-amide;
4-(1H-Indol-6-ylmethyl)-piperazine-1-carboxylic acid (2H-tetrazol-5-yl)-amide;
4-(3-Benzyloxy-benzyl)-piperazine-1-carboxylic acid (2H-tetrazol-5-yl)-amide;
4-Benzo[1,3]dioxol-5-ylmethyl-piperazine-1-carboxylic acid (2H-tetrazol-5-yl)-amide;
4-(3-Phenoxy-benzyl)-piperazine-1-carboxylic acid (2H-tetrazol-5-yl)-amide;
4-(3,4-Dichloro-benzyl)-piperazine-1-carboxylic acid (2H-tetrazol-5-yl)-amide;
4-Benzo[b]thiophen-2-ylmethyl-piperazine-1-carboxylic acid (2H-tetrazol-5-yl)-amide;
4-[3-(3-Cyano-phenoxy)-benzyl]-piperazine-1-carboxylic acid (2H-tetrazol-5-yl)-amide;
4-{3-[4-Fluoro-3-(trifluoromethyl)phenoxy]benzyl}-N-2H-tetrazol-5-ylpiperazine-1-carboxamide;
N-2H-Tetrazol-5-yl-4-{3-[3-(trifluoromethyl)phenoxy]benzyl}piperazine-1-carboxamide;
4-[3-(4-Cyanophenoxy)benzyl]-N-2H-tetrazol-5-ylpiperazine-1-carboxamide;
N-2H-Tetrazol-5-yl-4-{3-[4-(2,2,2-trifluoroethoxy)phenoxy]benzyl}-piperazine-1-carboxamide;
4-{3-[(2,2-Difluoro-1,3-benzodioxol-5-yl)oxy]benzyl}-N-2H -tetrazol-5-ylpiperazine-1-carboxamide;
4-[3-(2-Chlorophenoxy)benzyl]-N-2H-tetrazol-5-ylpiperazine-1-carboxamide;
4-[3-(4-Chloro-phenoxy)-benzyl]-piperazine-1-carboxylic acid (1,5-dimethyl-1H-pyrazol-3-yl)-amide;
4-[3-(4-Chloro-phenoxy)-benzyl]-piperazine-1-carboxylic acid (4-bromo-1-methyl-1H-pyrazol-3-yl)-amide;
4-[3-(4-Chloro-phenoxy)-benzyl]-piperazine-1-carboxylic acid (2-ethyl-2H-pyrazol-3-yl)-amide;
4-[3-(4-Chlorophenoxy)benzyl]-N-(5-methyl-1H-pyrazol-3-yl)piperazine-1-carboxamide;
4-(3,4-Dibromobenzyl)-N-pyridazin-3-ylpiperazine-1-carboxamide;
4-[(2,2-Difluoro-1,3-benzodioxol-5-yl)methyl]-N-pyridazin-3-ylpiperazine-1-carboxamide;
N-Pyridazin-3-yl-4-(quinolin-3-ylmethyl)piperazine-1-carboxamide;
N-Pyridazin-3-yl-4-(quinolin-2-ylmethyl)piperazine-1-carboxamide;
4-(3,4-Dichlorobenzyl)-N-pyridazin-3-ylpiperazine-1-carboxamide;
4-(Naphthalen-2-ylmethyl)-N-pyridazin-3-ylpiperazine-1-carboxamide;
4-(1H-Indol-5-ylmethyl)-N-pyridazin-3-ylpiperazine-1-carboxamide;
N-2,1,3-Benzothiadiazol-4-yl-4-{[3-(phenylethynyl)phenyl]methyl}-piperazine-1-carboxamide;
N-2,1,3-Benzoxadiazol-4-yl-4-{[3-(phenylethynyl)phenyl]methyl}-piperazine-1-carboxamide;
4-[3-(3-Chloro-4-trifluoromethyl-phenoxy)-benzyl]-piperazine-1-carboxylic acid benzo[d]isoxazol-3-ylamide;
4-[3-(4-Chloro-3-trifluoromethyl-phenoxy)-benzyl]-piperazine-1-carboxylic acid benzo[d]isoxazol-3-ylamide;
4-[3-(4-Chloro-3-fluoro-phenoxy)-benzyl]-piperazine-1-carboxylic acid benzo[d]isoxazol-3-ylamide;
4-[3-(3-Chloro-4-fluoro-phenoxy)-benzyl]-piperazine-1-carboxylic acid benzo[d]isoxazol-3-ylamide;
4-[3-(4-Fluoro-phenoxy)-benzyl]-piperazine-1-carboxylic acid benzo[d]isoxazol-3-ylamide;
4-[3-(4-Butyl-phenoxy)-benzyl]-piperazine-1-carboxylic acid benzo[d]isoxazol-3-ylamide;
4-[(2,2-Difluoro-1,3-benzodioxol-5-yl)methyl]-N-pyrazin-2-ylpiperazine-1-carboxamide;
4-(1,3-Benzodioxol-5-ylmethyl)-N-pyrazin-2-ylpiperazine-1-carboxamide;
4-(4-Bromobenzyl)-N-pyrazin-2-ylpiperazine-1-carboxamide;
4-(Naphthalen-2-ylmethyl)-N-pyrazin-2-ylpiperazine-1-carboxamide;
N-Pyrazin-2-yl-4-{3-[4-(2,2,2-trifluoroethoxy)phenoxy]benzyl}-piperazine-1-carboxamide;
N-Pyrazin-2-yl-4-{3-[4-(trifluoromethyl)phenoxy]benzyl}piperazine-1-carboxamide;
4-(1H-Indol-5-ylmethyl)-N-pyrazin-2-ylpiperazine-1-carboxamide;
4-(3,4-Dibromobenzyl)-N-pyrazin-2-ylpiperazine-1-carboxamide;
4-(1-Benzothiophen-2-ylmethyl)-N-pyrazin-2-ylpiperazine-1-carboxamide;
4-[4-(Benzyloxy)benzyl]-N-pyrazin-2-ylpiperazine-1-carboxamide;
4-(3,4-Dichlorobenzyl)-N-pyrazin-2-ylpiperazine-1-carboxamide;
4-[3-(4-Bromophenoxy)benzyl]-N-pyrazin-2-ylpiperazine-1-carboxamide;
4-(4-Bromo-3-fluorobenzyl)-N-pyrazin-2-ylpiperazine-1-carboxamide;
4-[3-(Benzyloxy)benzyl]-N-pyrazin-2-ylpiperazine-1-carboxamide;
N-Pyrazin-2-yl-4-(quinolin-3-ylmethyl)piperazine-1-carboxamide;
4-[3-(3-Chlorophenoxy)benzyl]-N-pyrazin-2-ylpiperazine-1-carboxamide;
N-Pyrazin-2-yl-4-(3-{4-[(trifluoromethyl)sulfonyl]phenoxy}benzyl)-piperazine-1-carboxamide;
4-(3-Phenoxy-benzyl)-piperazine-1-carboxylic acid pyrazin-2-ylamide;
4-[3-(Naphthalen-2-yloxy)-benzyl]-piperazine-1-carboxylic acid pyrazin-2-ylamide;
4-[3-(4-Cyano-phenoxy)-benzyl]-piperazine-1-carboxylic acid pyrazin-2-ylamide;
4-Benzofuran-2-ylmethyl-piperazine-1-carboxylic acid pyrazin-2-ylamide;
4-[3-(3,4-Difluorophenoxy)benzyl]-N-pyrazin-2-ylpiperazine-1-carboxamide;
N-Pyrazin-2-yl-4-[3-(quinolin-6-yloxy)benzyl]piperazine-1-carboxamide;
N-Pyrazin-2-yl-4-{3-[4-(trifluoromethoxy)phenoxy]benzyl}piperazine-1-carboxamide;
N-Pyrazin-2-yl-4-(3-{4-[(trifluoromethyl)sulfanyl]phenoxy}benzyl)-piperazine-1-carboxamide;
4-[3-(3-Cyanophenoxy)benzyl]-N-pyrazin-2-ylpiperazine-1-carboxamide;
4-{3-[4-Cyano-3-(trifluoromethyl)phenoxy]benzyl}-N-pyrazin-2-ylpiperazine-1-carboxamide;
4-{3-[(2,2-Difluoro-1,3-benzodioxol-5-yl)oxy]benzyl}-N-pyrazin-2-ylpiperazine-1-carboxamide;
4-[3-(2-Chlorophenoxy)benzyl]-N-pyrazin-2-ylpiperazine-1-carboxamide;
N-Pyrazin-2-yl-4-(quinolin-2-ylmethyl)piperazine-1-carboxamide;
4-[3-(3-Bromophenoxy)benzyl]-N-pyrazin-2-ylpiperazine-1-carboxamide;
4-{3-[4-Fluoro-3-(trifluoromethyl)phenoxy]benzyl}-N-pyrazin-2-ylpiperazine-1-carboxamide;
N-Pyrazin-2-yl-4-{3-[3-(trifluoromethoxy)phenoxy]benzyl}piperazine-1-carboxamide;
4-[3-(4-Chlorophenoxy)benzyl]-N-(3-chloropyrazin-2-yl)piperazine-1-carboxamide;
4-[3-(4-Chlorophenoxy)benzyl]-N-(5-phenyl-1H-pyrazol-3-yl)piperazine-1-carboxamide;
4-[3-(4-Chloro-phenoxy)-benzyl]-piperazine-1-carboxylic acid (6-fluoro-benzo[d]isoxazol-3-yl)-amide; and
4-[3-(4-Chloro-phenoxy)-benzyl]-piperazine-1-carboxylic acid pyridazin-3-ylamide;
and pharmaceutically acceptable salts thereof.

30. A pharmaceutical composition for treating a disease, disorder, or medical condition mediated by FAAH activity, comprising:
(a) an effective amount of at least one active agent selected from the group consisting of:
compounds of Formula (I): 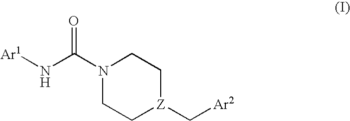
wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, 1H-pyrazol-3-yl, 5-methylpyrazin-2-yl, 3-chloropyrazin-2-yl, pyridazin-3-yl, 6-methoxypyridazin-3-yl, 5-methylisoxazol-3-yl, 1,5-dimethyl-1H-pyrazol-3-yl, 4-bromo-1-methyl-1H-pyrazol-3-yl, 2-ethyl-2H-pyrazol-3-yl, 5-methyl-1H-pyrazol-3-yl, or 5-phenyl-1H-pyrazol-3-yl group;
Z is —N— or >CH; and
Ar² is:
(i) phenyl unsubstituted or substituted with one or two R^{a} moieties;
where each R^{a} moiety is independently —C₁₋₄alkyl, —C≡C—R^{d}, —OC₁₋₄alkyl, halo, —CF₃, —OCF₃, —OCH₂CF₃, —SCF₃, —S(O)₀₋₂C₁₋₄alkyl, —SO₂CF₃, —OSO₂C₁₋₄alkyl, —(CH₂)₀₋₁CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —(CH₂)₀₋₁CN;
or two adjacent R^{a} moieties taken together form —O(CH₂)₁₋₂O— or —OCF₂O—;
where R^{b} and R^{c} are each independently —H or —C₁₋₄alkyl; and
R^{d} is H, C₃₋₆cycloalkyl, or —CH₂NR^{e}R^{f};
where R^{e} and R^{f} are each independently H or C₁₋₄alkyl;
(ii) phenyl substituted at the 3- or 4-position with -L-Ar³, unsubstituted or substituted with one or two R^{a} moieties, wherein:
L is a linker selected from the group consisting of —(CH₂)₁₋₃—, —CH═CH—, —O—, —OCH₂—, —CH₂O—, —NH—, >NC₁₋₄alkyl, —S—, —C≡C—, —C(═O)—, and a covalent bond; and
Ar³ is:
(a) phenyl;
(b) naphthyl; or
(c) a monocyclic or bicyclic heteroaryl group; or
(iii) a 9- or 10-membered fused bicyclic heteroaryl group;
where when Ar¹ is 6-chloro-pyridazin-3-yl, isoxazol-3-yl, or 1H-pyrazol-3-yl,
then Ar² is not benzo[1,3]dioxol-5-yl or 2,2-difluoro-benzo[1,3]dioxol-5-yl; and pharmaceutically acceptable salts, pharmaceutically acceptable prodrugs, and pharmaceutically active metabolites of said compounds of Formula (I); and
(b) a pharmaceutically acceptable excipient.

31. A pharmaceutical composition according to claim 30, wherein said active agent is selected from the group consisting of:
N-1,2-Benzisoxazol-3-yl-4-[(2,2-difluoro-1,3-benzodioxol-5-yl)methyl]piperidine-1-carboxamide;
4-(3-o-Tolylethynyl-benzyl)-piperazine-1-carboxylic acid benzo[d]isoxazol-3-ylamide;
N-1,2-Benzisoxazol-3-yl-4-(3-{[2-(trifluoromethyl)-phenyl]-ethynyl}benzyl)-piperazine-1-carboxamide;
N-1,2-Benzisoxazol-3-yl-4-{3-[(2-methoxyphenyl)-ethynyl]-benzyl}-piperazine-1-carboxamide;
N-1,2-Benzisoxazol-3-yl-4-{3-[(2-fluorophenyl)ethynyl]-benzyl}-piperazine-1-carboxamide;
N-1,2-Benzisoxazol-3-yl-4-{3-[(2-bromophenyl)-ethynyl]benzyl}-piperazine-1-carboxamide;
4-(3-Ethynyl-benzyl)-piperazine-1-carboxylic acid benzo[d]isoxazol-3-ylamide;
N-1,2-Benzisoxazol-3-yl-4-{3-[3-(dimethylamino)prop-1-yn-1-yl]benzyl}-piperazine-1-carboxamide;
N-1,2-Benzisoxazol-3-yl-4-[3-(cyclohexylethynyl)benzyl]-piperazine-1-carboxamide;
N-1,2-Benzisoxazol-3-yl-4-[3-(cyclopentylethynyl)benzyl]-piperazine-1-carboxamide;
N-1,2-Benzisoxazol-3-yl-4-{3-[(2-chlorophenyl)-ethynyl]benzyl}-piperazine-1-carboxamide;
N-1,2-Benzisoxazol-3-yl-4-{3-[(3-chlorophenyl)ethynyl]benzyl}-piperazine-1-carboxamide;
N-1,2-Benzisoxazol-3-yl-4-{3-[(4-chlorophenyl)ethynyl]benzyl}-piperazine-1-carboxamide;
N-1,2-Benzisoxazol-3-yl-4-{3-[(3,4-dichlorophenyl)ethynyl]-benzyl}-piperazine-1-carboxamide;
N-1,2-Benzisoxazol-3-yl-4-[3-(cyclopropylethynyl)benzyl]-piperazine-1-carboxamide;
N-1,2-Benzisoxazol-3-yl-4-[3-(thiophen-3-ylethynyl)benzyl]-piperazine-1-carboxamide;
4-{3-[(2-Chlorophenyl)ethynyl]benzyl}-N-pyrazin-2-ylpiperazine-1-carboxamide;
4-{3-[(2-Chlorophenyl)ethynyl]benzyl}-N-pyridazin-3-ylpiperazine-1-carboxamide;
4-{3-[(2-Chlorophenyl)ethynyl]benzyl}-N-(5-methylpyrazin-2-yl)piperazine-1-carboxamide;
N-1,2-Benzisoxazol-3-yl-4-{3-[(2,4-dichlorophenyl)-ethynyl]benzyl}-piperazine-1-carboxamide;
N-1,2-Benzisoxazol-3-yl-4-(3-{[2-(trifluoromethoxy)phenyl]-ethynyl}benzyl)-piperazine-1-carboxamide;
N-1,2-Benzisoxazol-3-yl-4-{3-[(3,5-dichlorophenyl)ethynyl]benzyl}-piperazine-1-carboxamide;
N-1,2-Benzisoxazol-3-yl-4-{3-[(2,5-dichlorophenyl)ethynyl]benzyl}-piperazine-1-carboxamide;
N-1,2-Benzisoxazol-3-yl-4-{3-[(2-cyanophenyl)ethynyl]benzyl}-piperazine-1-carboxamide;
N-1,2-Benzisoxazol-3-yl-4-[3-(naphthalen-1-ylethynyl)benzyl]piperazine-1-carboxamide;
Methyl 2-[(3-{[4-(1,2-benzisoxazol-3-ylcarbamoyl)piperazin-1-yl]methyl}phenyl)ethynyl]benzoate;
N-1,2-Benzisoxazol-3-yl-4-{3-[(3-cyanophenyl)ethynyl]benzyl}piperazine-1-carboxamide;
N-1,2-Benzisoxazol-3-yl-4-[3-(1,3-benzodioxol-5-ylethynyl)benzyl]-piperazine-1-carboxamide;
N-1,2-Benzisoxazol-3-yl-4-{3-[(2,3-dichlorophenyl)ethynyl]benzyl}-piperazine-1-carboxamide;
N-1,2-Benzisoxazol-3-yl-4-{3-[(2-cyano-3-fluorophenyl)ethynyl]-benzyl}piperazine-1-carboxamide;
N-1,2-Benzisoxazol-3-yl-4-(3-{[2-(cyanomethyl)phenyl]ethynyl}-benzyl)piperazine-1-carboxamide;
Methyl {2-[(3-{[4-(1,2-benzisoxazol-3-ylcarbamoyl)piperazin-1-yl]methyl}phenyl)ethynyl]phenyl}acetate;
4-[3-(2-o-Tolyl-ethyl)-benzyl]-piperazine-1-carboxylic acid benzo[d]isoxazol-3-ylamide;
4-[3-(Pyrimidin-2-yloxy)-benzyl]-piperazine-1-carboxylic acid benzo[d]isoxazol-3-ylamide;
N-1,2-Benzisoxazol-3-yl-4-[3-(pyridin-2-yloxy)benzyl]piperazine-1-carboxamide;
N-1,2-Benzisoxazol-3-yl-4-[3-(pyrazin-2-yloxy)benzyl]piperazine-1-carboxamide;
4-[3-(2-Cyano-benzyloxy)-benzyl]-piperazine-1-carboxylic acid benzo[d]isoxazol-3-ylamide;
N-1,2-Benzisoxazol-3-yl-4-[3-(benzyloxy)benzyl]piperazine-1-carboxamide;
4-(1H-Benzimidazol-6-ylmethyl)-N-1,2-benzisoxazol-3-ylpiperazine-1-carboxamide;
N-1,2-Benzisoxazol-3-yl-4-(1H-indazol-6-ylmethyl)piperazine-1-carboxamide;
N-1,2-Benzisoxazol-3-yl-4-[4-(methylsulfonyl)benzyl]piperazine-1-carboxamide;
N-1,2-Benzisoxazol-3-yl-4-[4-(trifluoromethoxy)benzyl]piperazine-1-carboxamide;
4-[3-(4-Chlorophenoxy)benzyl]-N-(6-methoxypyridazin-3-yl)piperazine-1-carboxamide;
N-1,2-Benzisoxazol-3-yl-4-[4-chloro-3-(trifluoromethoxy)benzyl]-piperazine-1-carboxamide;
N-1,2-Benzisoxazol-3-yl-4-[4-fluoro-3-(trifluoromethoxy)benzyl]-piperazine-1-carboxamide;
N-1,2-Benzisoxazol-3-yl-4-[3-chloro-4-(trifluoromethoxy)benzyl]-piperazine-1-carboxamide;
N-1,2-Benzisoxazol-3-yl-4-[3-fluoro-4-(trifluoromethoxy)benzyl]-piperazine-1-carboxamide;
N-1,2-Benzisoxazol-3-yl-4-{3-[4-(trifluoromethyl)phenoxy]benzyl}-piperazine-1-carboxamide;
N-1,2-Benzisoxazol-3-yl-4-(3-phenoxybenzyl)piperazine-1-carboxamide;
N-1,2-Benzisoxazol-3-yl-4-(3,4-dichlorobenzyl)piperazine-1-carboxamide;
N-1,2-Benzisoxazol-3-yl-4-[4-(benzyloxy)benzyl]piperazine-1-carboxamide;
N-1,2-Benzisoxazol-3-yl-4-(1-benzothiophen-2-ylmethyl)piperazine-1-carboxamide;
N-1,2-Benzisoxazol-3-yl-4-[3-(quinolin-6-yloxy)benzyl]piperazine-1-carboxamide;
N-1,2-Benzisoxazol-3-yl-4-(4-bromo-3-fluorobenzyl)piperazine-1-carboxamide;
N-1,2-Benzisoxazol-3-yl-4-(1,3-benzodioxol-5-ylmethyl)piperazine-1-carboxamide;
N-1,2-Benzisoxazol-3-yl-4-(quinolin-3-ylmethyl)piperazine-1-carboxamide;
N-1,2-Benzisoxazol-3-yl-4-(1H-indol-5-ylmethyl)piperazine-1-carboxamide;
N-1,2-Benzisoxazol-3-yl-4-[3-(naphthalen-2-yloxy)benzyl]piperazine-1-carboxamide;
N-1,2-Benzisoxazol-3-yl-4-(4-bromobenzyl)piperazine-1-carboxamide;
N-1,2-Benzisoxazol-3-yl-4-(3,4-dibromobenzyl)piperazine-1-carboxamide;
N-1,2-Benzisoxazol-3-yl-4-[3-(2-chlorophenoxy)benzyl]piperazine-1-carboxamide;
4-Naphthalen-2-ylmethyl-piperazine-1-carboxylic acid benzo[d]isoxazol-3-ylamide;
4-Quinolin-2-ylmethyl-piperazine-1-carboxylic acid benzo[d]isoxazol-3-ylamide;
4-[3-(4-Cyano-phenoxy)-benzyl]-piperazine-1-carboxylic acid benzo[d]isoxazol-3-ylamide;
4-Benzofuran-2-ylmethyl-piperazine-1-carboxylic acid benzo[d]isoxazol-3-ylamide;
N-1,2-Benzisoxazol-3-yl-4-[3-(3-chlorophenoxy)benzyl]piperazine-1-carboxamide;
N-1,2-Benzisoxazol-3-yl-4-{3-[4-cyano-3-(trifluoromethyl)phenoxy]benzyl}piperazine-1-carboxamide;
N-1,2-Benzisoxazol-3-yl-4-[3-(3-cyanophenoxy)benzyl]piperazine-1-carboxamide;
N-1,2-Benzisoxazol-3-yl-4-(3-{4-[(trifluoromethyl)sulfanyl]phenoxy}-benzyl)piperazine-1-carboxamide;
N-1,2-Benzisoxazol-3-yl-4-{3-[(2,2-difluoro-1,3-benzodioxol-5-yl)oxy]benzyl}piperazine-1-carboxamide;
N-1,2-Benzisoxazol-3-yl-4-(3-{4-[(trifluoromethyl)sulfonyl]phenoxy}-benzyl)piperazine-1-carboxamide;
N-1,2-Benzisoxazol-3-yl-4-{[3-(phenylethynyl)phenyl]methyl}piperazine-1-carboxamide;
N-Isoxazol-3-yl-4-{3-[4-(trifluoromethoxy)phenoxy]benzyl}piperazine-1-carboxamide;
4-[4-(Benzyloxy)benzyl]-N-isoxazol-3-ylpiperazine-1-carboxamide;
4-[3-(3-Chlorophenoxy)benzyl]-N-isoxazol-3-ylpiperazine-1-carboxamide;
N-isoxazol-3-yl-4-{3-[4-(2,2,2-trifluoroethoxy)phenoxy]benzyl}piperazine-1-carboxamide;
4-(1-Benzofuran-2-ylmethyl)-N-isoxazol-3-ylpiperazine-1-carboxamide;
4-[3-(3-Cyanophenoxy)benzyl]-N-isoxazol-3-ylpiperazine-1-carboxamide;
4-[3-(2-Chlorophenoxy)benzyl]-N-isoxazol-3-ylpiperazine-1-carboxamide;
4-{3-[(2,2-Difluoro-1,3-benzodioxol-5-yl)oxy]benzyl}-N-isoxazol-3-ylpiperazine-1-carboxamide;
4-(1-Benzothiophen-2-ylmethyl)-N-isoxazol-3-ylpiperazine-1-carboxamide;
4-(1,3-Benzodioxol-5-ylmethyl)-N-isoxazol-3-ylpiperazine-1-carboxamide;
N-Isoxazol-3-yl-4-(naphthalen-2-ylmethyl)piperazine-1-carboxamide;
4-[3-(4-Bromophenoxy)benzyl]-N-isoxazol-3-ylpiperazine-1-carboxamide;
4-Quinolin-2-ylmethyl-piperazine-1-carboxylic acid isoxazol-3-ylamide;
4-Quinolin-3-ylmethyl-piperazine-1-carboxylic acid isoxazol-3-ylamide;
4-(4-Bromo-benzyl)-piperazine-1-carboxylic acid isoxazol-3-ylamide;
4-(1H-Indol-6-ylmethyl)-piperazine-1-carboxylic acid isoxazol-3-ylamide;
4-[3-(Naphthalen-2-yloxy)-benzyl]-piperazine-1-carboxylic acid isoxazol-3-ylamide;
4-(4-Bromo-3-fluoro-benzyl)-piperazine-1-carboxylic acid isoxazol-3-ylamide;
4-[3-(4-Cyano-phenoxy)-benzyl]-piperazine-1-carboxylic acid isoxazol-3-ylamide;
4-[3-(3,4-Difluorophenoxy)benzyl]-N-isoxazol-3-ylpiperazine-1-carboxamide;
4-(3,4-Dibromobenzyl)-N-isoxazol-3-ylpiperazine-1-carboxamide;
N-Isoxazol-3-yl-4-(3-{4-[(trifluoromethyl)sulfanyl]phenoxy}benzyl)-piperazine-1-carboxamide;
4-{3-[4-Fluoro-3-(trifluoromethyl)phenoxy]benzyl}-N-isoxazol-3-ylpiperazine-1-carboxamide;
4-[3-(3-Bromophenoxy)benzyl]-N-isoxazol-3-ylpiperazine-1-carboxamide;
N-Isoxazol-3-yl-4-(3-{4-[(trifluoromethyl)sulfonyl]phenoxy}benzyl)-piperazine-1-carboxamide;
N-Isoxazol-3-yl-4-{3-[3-(trifluoromethoxy)phenoxy]benzyl}piperazine-1-carboxamide;
4-(3,4-Dichlorobenzyl)-N-isoxazol-3-ylpiperazine-1-carboxamide;
N-Isoxazol-3-yl-4-{3-[4-(trifluoromethyl)phenoxy]benzyl}piperazine-1-carboxamide;
N-Isoxazol-3-yl-4-[3-(quinolin-6-yloxy)benzyl]piperazine-1-carboxamide;
4-{3-[4-Cyano-3-(trifluoromethyl)phenoxy]benzyl}-N-isoxazol-3-ylpiperazine-1-carboxamide;
4-[3-(4-Chlorophenoxy)benzyl]-N-(5-methylisoxazol-3-yl)piperazine-1-carboxamide;
4-(Quinolin-3-ylmethyl)-N-1H-tetrazol-5-ylpiperazine-1-carboxamide;
4-[3-(Naphthalen-2-yloxy)benzyl]-N-1H-tetrazol-5-ylpiperazine-1-carboxamide;
4-(3,4-Dibromobenzyl)-N-1H-tetrazol-5-ylpiperazine-1-carboxamide;
4-(4-Bromo-3-fluorobenzyl)-N-1H-tetrazol-5-ylpiperazine-1-carboxamide;
4-[3-(3,4-Difluorophenoxy)benzyl]-N-1H-tetrazol-5-ylpiperazine-1-carboxamide;
4-{3-[4-Cyano-3-(trifluoromethyl)phenoxy]benzyl}-N-1H-tetrazol-5-ylpiperazine-1-carboxamide;
N-1H-Tetrazol-5-yl-4-{3-[4-(trifluoromethyl)phenoxy]benzyl}piperazine-1-carboxamide;
N-1H-Tetrazol-5-yl-4-(3-{4-[(trifluoromethyl)sulfanyl]phenoxy}benzyl)-piperazine-1-carboxamide;
N-1H-Tetrazol-5-yl-4-{3-[3-(trifluoromethoxy)phenoxy]benzyl}piperazine-1-carboxamide;
4-[3-(3,4-Dichlorophenoxy)benzyl]-N-1H-tetrazol-5-ylpiperazine-1-carboxamide;
4-(Quinolin-2-ylmethyl)-N-1H-tetrazol-5-ylpiperazine-1-carboxamide;
4-(Naphthalen-2-ylmethyl)-N-1H-tetrazol-5-ylpiperazine-1-carboxamide;
4-(4-Bromo-benzyl)-piperazine-1-carboxylic acid (2H-tetrazol-5-yl)-amide;
4-(1H-Indol-6-ylmethyl)-piperazine-1-carboxylic acid (2H-tetrazol-5-yl)-amide;
4-(3-Benzyloxy-benzyl)-piperazine-1-carboxylic acid (2H-tetrazol-5-yl)-amide;
4-Benzo[1,3]dioxol-5-ylmethyl-piperazine-1-carboxylic acid (2H-tetrazol-5-yl)-amide;
4-(3-Phenoxy-benzyl)-piperazine-1-carboxylic acid (2H-tetrazol-5-yl)-amide;
4-(3,4-Dichloro-benzyl)-piperazine-1-carboxylic acid (2H-tetrazol-5-yl)-amide;
4-Benzo[b]thiophen-2-ylmethyl-piperazine-1-carboxylic acid (2H-tetrazol-5-yl)-amide;
4-[3-(3-Cyano-phenoxy)-benzyl]-piperazine-1-carboxylic acid (2H-tetrazol-5-yl)-amide;
4-{3-[4-Fluoro-3-(trifluoromethyl)phenoxy]benzyl}-N-2H-tetrazol-5-ylpiperazine-1-carboxamide;
N-2H-Tetrazol-5-yl-4-{3-[3-(trifluoromethyl)phenoxy]benzyl}piperazine-1-carboxamide;
4-[3-(4-Cyanophenoxy)benzyl]-N-2H-tetrazol-5-ylpiperazine-1-carboxamide;
N-2H-Tetrazol-5-yl-4-{3-[4-(2,2,2-trifluoroethoxy)phenoxy]benzyl}-piperazine-1-carboxamide;
4-{3-[(2,2-Difluoro-1,3-benzodioxol-5-yl)oxy]benzyl}-N-2H-tetrazol-5-ylpiperazine-1-carboxamide;
4-[3-(2-Chlorophenoxy)benzyl]-N-2H-tetrazol-5-ylpiperazine-1-carboxamide;
4-[3-(4-Chloro-phenoxy)-benzyl]-piperazine-1-carboxylic acid (1,5-dimethyl-1H-pyrazol-3-yl)-amide;
4-[3-(4-Chloro-phenoxy)-benzyl]-piperazine-1-carboxylic acid (4-bromo-1-methyl-1H-pyrazol-3-yl)-amide;
4-[3-(4-Chloro-phenoxy)-benzyl]-piperazine-1-carboxylic acid (2-ethyl-2H-pyrazol-3-yl)-amide;
4-[3-(4-Chlorophenoxy)benzyl]-N-(5-methyl-1H-pyrazol-3-yl)piperazine-1-carboxamide;
4-(3,4-Dibromobenzyl)-N-pyridazin-3-ylpiperazine-1-carboxamide;
4-[(2,2-Difluoro-1,3-benzodioxol-5-yl)methyl]-N-pyridazin-3-ylpiperazine-1-carboxamide;
N-Pyridazin-3-yl-4-(quinolin-3-ylmethyl)piperazine-1-carboxamide;
N-Pyridazin-3-yl-4-(quinolin-2-ylmethyl)piperazine-1-carboxamide;
4-(3,4-Dichlorobenzyl)-N-pyridazin-3-ylpiperazine-1-carboxamide;
4-(Naphthalen-2-ylmethyl)-N-pyridazin-3-ylpiperazine-1-carboxamide;
4-(1H-Indol-5-ylmethyl)-N-pyridazin-3-ylpiperazine-1-carboxamide;
N-2,1,3-Benzothiadiazol-4-yl-4-{[3-(phenylethynyl)phenyl]methyl}-piperazine-1-carboxamide;
N-2,1,3-Benzoxadiazol-4-yl-4-{[3-(phenylethynyl)phenyl]methyl}-piperazine-1-carboxamide;
4-[3-(3-Chloro-4-trifluoromethyl-phenoxy)-benzyl]-piperazine-1-carboxylic acid benzo[d]isoxazol-3-ylamide;
4-[3-(4-Chloro-3-trifluoromethyl-phenoxy)-benzyl]-piperazine-1-carboxylic acid benzo[d]isoxazol-3-ylamide;
4-[3-(4-Chloro-3-fluoro-phenoxy)-benzyl]-piperazine-1-carboxylic acid benzo[d]isoxazol-3-ylamide;
4-[3-(3-Chloro-4-fluoro-phenoxy)-benzyl]-piperazine-1-carboxylic acid benzo[d]isoxazol-3-ylamide;
4-[3-(4-Fluoro-phenoxy)-benzyl]-piperazine-1-carboxylic acid benzo[d]isoxazol-3-ylamide;
4-[3-(4-Butyl-phenoxy)-benzyl]-piperazine-1-carboxylic acid benzo[d]isoxazol-3-ylamide;
4-[(2,2-Difluoro-1,3-benzodioxol-5-yl)methyl]-N-pyrazin-2-ylpiperazine-1-carboxamide;
4-(1,3-Benzodioxol-5-ylmethyl)-N-pyrazin-2-ylpiperazine-1-carboxamide;
4-(4-Bromobenzyl)-N-pyrazin-2-ylpiperazine-1-carboxamide;
4-(Naphthalen-2-ylmethyl)-N-pyrazin-2-ylpiperazine-1-carboxamide;
N-Pyrazin-2-yl-4-{3-[4-(2,2,2-trifluoroethoxy)phenoxy]benzyl}-piperazine-1-carboxamide;
N-Pyrazin-2-yl-4-{3-[4-(trifluoromethyl)phenoxy]benzyl}piperazine-1-carboxamide;
4-(1H-Indol-5-ylmethyl)-N-pyrazin-2-ylpiperazine-1-carboxamide;
4-(3,4-Dibromobenzyl)-N-pyrazin-2-ylpiperazine-1-carboxamide;
4-(1-Benzothiophen-2-ylmethyl)-N-pyrazin-2-ylpiperazine-1-carboxamide;
4-[4-(Benzyloxy)benzyl]-N-pyrazin-2-ylpiperazine-1-carboxamide;
4-(3,4-Dichlorobenzyl)-N-pyrazin-2-ylpiperazine-1-carboxamide;
4-[3-(4-Bromophenoxy)benzyl]-N-pyrazin-2-ylpiperazine-1-carboxamide;
4-(4-Bromo-3-fluorobenzyl)-N-pyrazin-2-ylpiperazine-1-carboxamide;
4-[3-(Benzyloxy)benzyl]-N-pyrazin-2-ylpiperazine-1-carboxamide;
N-Pyrazin-2-yl-4-(quinolin-3-ylmethyl)piperazine-1-carboxamide;
4-[3-(3-Chlorophenoxy)benzyl]-N-pyrazin-2-ylpiperazine-1-carboxamide;
N-Pyrazin-2-yl-4-(3-{4-[(trifluoromethyl)sulfonyl]phenoxy}benzyl)-piperazine-1-carboxamide;
4-(3-Phenoxy-benzyl)-piperazine-1-carboxylic acid pyrazin-2-ylamide;
4-[3-(Naphthalen-2-yloxy)-benzyl]-piperazine-1-carboxylic acid pyrazin-2-ylamide;
4-[3-(4-Cyano-phenoxy)-benzyl]-piperazine-1-carboxylic acid pyrazin-2-ylamide;
4-Benzofuran-2-ylmethyl-piperazine-1-carboxylic acid pyrazin-2-ylamide;
4-[3-(3,4-Difluorophenoxy)benzyl]-N-pyrazin-2-ylpiperazine-1-carboxamide;
N-Pyrazin-2-yl-4-[3-(quinolin-6-yloxy)benzyl]piperazine-1-carboxamide;
N-Pyrazin-2-yl-4-{3-[4-(trifluoromethoxy)phenoxy]benzyl}piperazine-1-carboxamide;
N-Pyrazin-2-yl-4-(3-{4-[(trifluoromethyl)sulfanyl]phenoxy}benzyl)-piperazine-1-carboxamide;
4-[3-(3-Cyanophenoxy)benzyl]-N-pyrazin-2-ylpiperazine-1-carboxamide;
4-{3-[4-Cyano-3-(trifluoromethyl)phenoxy]benzyl}-N-pyrazin-2-ylpiperazine-1-carboxamide;
4-{3-[(2,2-Difluoro-1,3-benzodioxol-5-yl)oxy]benzyl}-N-pyrazin-2-ylpiperazine-1-carboxamide;
4-[3-(2-Chlorophenoxy)benzyl]-N-pyrazin-2-ylpiperazine-1-carboxamide;
N-Pyrazin-2-yl-4-(quinolin-2-ylmethyl)piperazine-1-carboxamide;
4-[3-(3-Bromophenoxy)benzyl]-N-pyrazin-2-ylpiperazine-1-carboxamide;
4-{3-[4-Fluoro-3-(trifluoromethyl)phenoxy]benzyl}-N-pyrazin-2-ylpiperazine-1-carboxamide;
N-Pyrazin-2-yl-4-{3-[3-(trifluoromethoxy)phenoxy]benzyl}piperazine-1-carboxamide;
4-[3-(4-Chlorophenoxy)benzyl]-N-(3-chloropyrazin-2-yl)piperazine-1-carboxamide;
4-[3-(4-Chlorophenoxy)benzyl]-N-(5-phenyl-1H-pyrazol-3-yl)piperazine-1-carboxamide;
4-[3-(4-Chloro-phenoxy)-benzyl]-piperazine-1-carboxylic acid (6-fluoro-benzo[d]isoxazol-3-yl)-amide; and
4-[3-(4-Chloro-phenoxy)-benzyl]-piperazine-1-carboxylic acid pyridazin-3-ylamide;
and pharmaceutically acceptable salts thereof.

32. A pharmaceutical composition according to claim 30, further comprising: an analgesic selected from the group consisting of opioids and non-steroidal anti-inflammatory drugs.

33. A pharmaceutical composition according to claim 30, further comprising: an additional active ingredient selected from the group consisting of aspirin, acetaminophen, opioids, ibuprofen, naproxen, COX-2 inhibitors, gabapentin, pregabalin, and tramadol.

34. A pharmaceutical composition for treating a disease, disorder, or medical condition mediated by FAAH activity, comprising:
(a) an effective amount of at least one active agent selected from the group consisting of:
compounds of Formula (Ia): 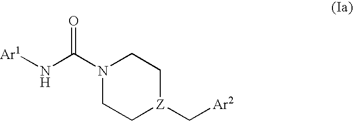
wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, or 1H-pyrazol-3-yl group;
Z is —N— or >CH; and
Ar² is:
(i) phenyl or 3-phenoxyphenyl substituted with one or two R^{a} moieties;
where each R^{a} moiety is independently —C₁₋₄alkyl, —OC₁₋₄alkyl, halo, —CF₃, —OCF₃, —OCH₂CF₃, —SCF₃, —S(O)₀₋₂C₁₋₄alkyl, —OSO₂C₁₋₄alkyl, —CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —CN;
where R^{b} and R^{c} are each independently —H or —C₁₋₄alkyl; or
(ii) benzo[1,3]dioxol-5-yl, 2,2-difluoro-benzo[1,3]dioxol-5-yl, or naphthyl;
where when Ar¹ is 6-chloro-pyridazin-3-yl, isoxazol-3-yl, or 1H-pyrazol-3-yl,
then Ar² is not benzo[1,3]dioxol-5-yl or 2,2-difluoro-benzo[1,3]dioxol-5-yl; and pharmaceutically acceptable salts, pharmaceutically acceptable prodrugs, and pharmaceutically active metabolites of said compounds of Formula (Ia); and
(b) a pharmaceutically acceptable excipient.

35. A pharmaceutical composition according to claim 34, wherein said active agent is selected from the group consisting of:
4-(2,2-Difluoro-benzo[1,3]dioxol-5-ylmethyl)-piperazine-1-carboxylic acid benzo[d]isoxazol-3-ylamide;
4-(2,2-Difluoro-benzo[1,3]dioxol-5-ylmethyl)-piperazine-1-carboxylic acid (3-phenyl-[1,2,4]thiadiazol-5-yl)-amide;
4-(2,2-Difluoro-benzo[1,3]dioxol-5-ylmethyl)-piperazine-1-carboxylic acid (1H-tetrazol-5-yl)-amide;
4-(2,2-Difluoro-benzo[1,3]dioxol-5-ylmethyl)-piperazine-1-carboxylic acid benzo[1,2,5]thiadiazol-4-ylamide;
4-(2,2-Difluoro-benzo[1,3]dioxol-5-ylmethyl)-piperazine-1-carboxylic acid benzo[1,2,5]oxadiazol-4-ylamide;
4-(2,2-Difluoro-benzo[1,3]dioxol-5-ylmethyl)-piperazine-1-carboxylic acid (3H-benzotriazol-5-yl)-amide;
4-(2,2-Difluoro-benzo[1,3]dioxol-5-ylmethyl)-piperazine-1-carboxylic acid thiophen-2-ylamide;
4-(2,2-Difluoro-benzo[1,3]dioxol-5-ylmethyl)-piperazine-1-carboxylic acid thiophen-3-ylamide;
4-Naphthalen-2-ylmethyl-piperidine-1-carboxylic acid (6-chloro-pyridazin-3-yl)-amide;
4-Naphthalen-2-ylmethyl-piperidine-1-carboxylic acid pyrazin-2-ylamide;
4-Naphthalen-2-ylmethyl-piperidine-1-carboxylic acid isoxazol-3-ylamide;
4-Naphthalen-2-ylmethyl-piperidine-1-carboxylic acid (3-phenyl-[1,2,4]thiadiazol-5-yl)-amide;
4-Naphthalen-2-ylmethyl-piperidine-1-carboxylic acid (1H-tetrazol-5-yl)-amide;
4-Naphthalen-2-ylmethyl-piperidine-1-carboxylic acid (2H-pyrazol-3-yl)-amide;
4-Naphthalen-2-ylmethyl-piperidine-1-carboxylic acid benzo[1,2,5]oxadiazol-4-ylamide;
4-Naphthalen-2-ylmethyl-piperidine-1-carboxylic acid (1H-benzotriazol-5-yl)-amide;
4-Naphthalen-2-ylmethyl-piperidine-1-carboxylic acid [1,5]naphthyridin-2-ylamide;
4-Naphthalen-2-ylmethyl-piperidine-1-carboxylic acid quinolin-2-ylamide;
4-Naphthalen-2-ylmethyl-piperidine-1-carboxylic acid benzothiazol-6-ylamide;
4-Naphthalen-2-ylmethyl-piperidine-1-carboxylic acid quinolin-5-ylamide;
4-Naphthalen-2-ylmethyl-piperidine-1-carboxylic acid benzo[d]isoxazol-3-ylamide;
4-(4-Fluoro-benzyl)-piperidine-1-carboxylic acid benzo[d]isoxazol-3-ylamide;
4-(4-Fluoro-benzyl)-piperidine-1-carboxylic acid (6-chloro-pyridazin-3-yl)-amide;
4-(4-Fluoro-benzyl)-piperidine-1-carboxylic acid isoxazol-3-ylamide;
4-(3-Trifluoromethyl-benzyl)-piperidine-1-carboxylic acid (6-chloro-pyridazin-3-yl)-amide;
4-(3-Trifluoromethyl-benzyl)-piperidine-1-carboxylic acid isoxazol-3-ylamide;
4-(3-Trifluoromethyl-benzyl)-piperidine-1-carboxylic acid benzo[d]isoxazol-3-ylamide;
4-[3-(4-Fluoro-3-trifluoromethyl-phenoxy)-benzyl]-piperazine-1-carboxylic acid benzo[d]isoxazol-3-ylamide;
4-[3-(3-Trifluoromethoxy-phenoxy)-benzyl]-piperazine-1-carboxylic acid benzo[d]isoxazol-3-ylamide;
4-[3-(4-Trifluoromethoxy-phenoxy)-benzyl]-piperazine-1-carboxylic acid benzo[d]isoxazol-3-ylamide;
4-[3-(3-Bromo-phenoxy)-benzyl]-piperazine-1-carboxylic acid benzo[d]isoxazol-3-ylamide;
4-[3-(4-Bromo-phenoxy)-benzyl]-piperazine-1-carboxylic acid benzo[d]isoxazol-3-ylamide;
4-[3-(3,4-Difluoro-phenoxy)-benzyl]-piperazine-1-carboxylic acid benzo[d]isoxazol-3-ylamide;
4-[3-(3,5-Difluoro-phenoxy)-benzyl]-piperazine-1-carboxylic acid benzo[d]isoxazol-3-ylamide;
4-{3-[4-(2,2,2-Trifluoro-ethoxy)-phenoxy]-benzyl}-piperazine-1-carboxylic acid benzo[d]isoxazol-3-ylamide;
4-[3-(4-Chloro-phenoxy)-benzyl]-piperazine-1-carboxylic acid benzo[d]isoxazol-3-ylamide;
4-[3-(4-Chloro-phenoxy)-benzyl]-piperazine-1-carboxylic acid benzo[1,2,5]thiadiazol-4-ylamide;
4-[3-(3,4-Dichloro-phenoxy)-benzyl]-piperazine-1-carboxylic acid benzo[1,2,5]thiadiazol-4-ylamide;
4-[3-(3,5-Dichloro-phenoxy)-benzyl]-piperazine-1-carboxylic acid benzo[1,2,5]thiadiazol-4-ylamide;
4-[3-(3-Trifluoromethyl-phenoxy)-benzyl]-piperazine-1-carboxylic acid benzo[1,2,5]thiadiazol-4-ylamide;
4-(3-Trifluoromethoxy-benzyl)-piperazine-1-carboxylic acid benzo[1,2,5]thiadiazol-4-ylamide;
4-[3-(4-Chloro-phenoxy)-benzyl]-piperazine-1-carboxylic acid (6-chloro-pyridazin-3-yl)-amide;
4-[3-(3,4-Dichloro-phenoxy)-benzyl]-piperazine-1-carboxylic acid (6-chloro-pyridazin-3-yl)-amide;
4-[3-(3-Trifluoromethyl-phenoxy)-benzyl]-piperazine-1-carboxylic acid (6-chloro-pyridazin-3-yl)-amide;
4-(4-Fluoro-3-phenoxy-benzyl)-piperazine-1-carboxylic acid (6-chloro-pyridazin-3-yl)-amide;
4-[3-(4-Chloro-phenoxy)-benzyl]-piperazine-1-carboxylic acid isoxazol-3-ylamide;
4-[3-(3,4-Dichloro-phenoxy)-benzyl]-piperazine-1-carboxylic acid isoxazol-3-ylamide;
4-[3-(3-Trifluoromethyl-phenoxy)-benzyl]-piperazine-1-carboxylic acid isoxazol-3-ylamide;
4-(4-Fluoro-3-phenoxy-benzyl)-piperazine-1-carboxylic acid isoxazol-3-ylamide;
4-[3-(3,4-Dichloro-phenoxy)-benzyl]-piperazine-1-carboxylic acid benzo[d]isoxazol-3-ylamide;
4-[3-(3-Trifluoromethyl-phenoxy)-benzyl]-piperazine-1-carboxylic acid benzo[d]isoxazol-3-ylamide;
4-(3-Trifluoromethoxy-benzyl)-piperazine-1-carboxylic acid benzo[d]isoxazol-3-ylamide;
4-[3-(4-Chloro-phenoxy)-benzyl]-piperazine-1-carboxylic acid (1H-pyrazol-3-yl)-amide;
4-[3-(3,4-Dichloro-phenoxy)-benzyl]-piperazine-1-carboxylic acid (1H-pyrazol-3-yl)-amide;
4-[3-(3-Trifluoromethyl-phenoxy)-benzyl]-piperazine-1-carboxylic acid (1H-pyrazol-3-yl)-amide;
4-(4-Fluoro-3-phenoxy-benzyl)-piperazine-1-carboxylic acid (1H-pyrazol-3-yl)-amide;
4-[3-(4-Chloro-phenoxy)-benzyl]-piperazine-1-carboxylic acid (1H-tetrazol-5-yl)-amide;
4-[3-(4-Chloro-phenoxy)-benzyl]-piperazine-1-carboxylic acid pyrazin-2-ylamide;
4-[3-(3,4-Dichloro-phenoxy)-benzyl]-piperazine-1-carboxylic acid pyrazin-2-ylamide;
4-[3-(3-Trifluoromethyl-phenoxy)-benzyl]-piperazine-1-carboxylic acid pyrazin-2-ylamide; and
4-(3-Trifluoromethoxy-benzyl)-piperazine-1-carboxylic acid pyrazin-2-ylamide;
and pharmaceutically acceptable salts thereof.

36. A pharmaceutical composition according to claim 34, further comprising: an analgesic selected from the group consisting of opioids and non-steroidal anti-inflammatory drugs.

37. A pharmaceutical composition according to claim 34, further comprising: an additional active ingredient selected from the group consisting of aspirin, acetaminophen, opioids, ibuprofen, naproxen, COX-2 inhibitors, gabapentin, pregabalin, and tramadol.

38. A method of treating a subject suffering from or diagnosed with a disease, disorder, or medical condition mediated by FAAH activity, comprising administering to the subject in need of such treatment an effective amount of at least one active selected from compounds of Formula (I): 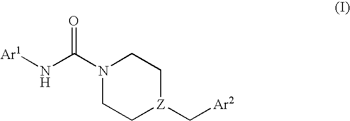
wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, 1H-pyrazol-3-yl, 5-methylpyrazin-2-yl, 3-chloropyrazin-2-yl, pyridazin-3-yl, 6-methoxypyridazin-3-yl, 5-methylisoxazol-3-yl, 1,5-dimethyl-1H-pyrazol-3-yl, 4-bromo-1-methyl-1H-pyrazol-3-yl, 2-ethyl-2H-pyrazol-3-yl, 5-methyl-1H-pyrazol-3-yl, or 5-phenyl-1H-pyrazol-3-yl group;
Z is —N— or >CH; and
Ar² is:
(i) phenyl unsubstituted or substituted with one or two R^{a} moieties;
where each R^{a} moiety is independently —C₁₋₄alkyl, —C≡C—R^{d}, —OC₁₋₄alkyl, halo, —CF₃, —OCF₃, —OCH₂CF₃, —SCF₃, —S(O)₀₋₂C₁₋₄alkyl, —SO₂CF₃, —OSO₂C₁₋₄alkyl, —(CH₂)₀₋₁CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —(CH₂)₀₋₁CN;
or two adjacent R^{a} moieties taken together form —O(CH₂)₁₋₂O— or —OCF₂O—;
where R^{b} and R^{c} are each independently —H or —C₁₋₄alkyl; and
R^{d} is H, C₃₋₆cycloalkyl, or —CH₂NR^{e}R^{f};
where R^{e} and R^{f} are each independently H or C₁₋₄alkyl;
(ii) phenyl substituted at the 3- or 4-position with -L-Ar³, unsubstituted or substituted with one or two R^{a} moieties, wherein:
L is a linker selected from the group consisting of —(CH₂)₁₋₃—, —CH═CH—, —O—, —OCH₂—, —CH₂O—, —NH—, >NC₁₋₄alkyl, —S—, —C≡C—, —C(═O)—, and
a covalent bond; and
Ar³ is:
(a) phenyl;
(b) naphthyl; or
(c) a monocyclic or bicyclic heteroaryl group; or
(iii) a 9- or 10-membered fused bicyclic heteroaryl group;
where when Ar¹ is 6-chloro-pyridazin-3-yl, isoxazol-3-yl, or 1H-pyrazol-3-yl,
then Ar² is not benzo[1,3]dioxol-5-yl or 2,2-difluoro-benzo[1,3]dioxol-5-yl;
and pharmaceutically acceptable salts, pharmaceutically acceptable prodrugs, and pharmaceutically active metabolites of said compounds of Formula (I).

39. A method according to claim 38, wherein said active agent is selected from the group consisting of:
N-1,2-Benzisoxazol-3-yl-4-[(2,2-difluoro-1,3-benzodioxol-5-yl)methyl]piperidine-1-carboxamide;
4-(3-o-Tolylethynyl-benzyl)-piperazine-1-carboxylic acid benzo[d]isoxazol-3-ylamide;
N-1,2-Benzisoxazol-3-yl-4-(3-{[2-(trifluoromethyl)-phenyl]-ethynyl}benzyl)-piperazine-1-carboxamide;
N-1,2-Benzisoxazol-3-yl-4-{3-[(2-methoxyphenyl)-ethynyl]-benzyl}-piperazine-1-carboxamide;
N-1,2-Benzisoxazol-3-yl-4-{3-[(2-fluorophenyl)ethynyl]-benzyl}-piperazine-1-carboxamide;
N-1,2-Benzisoxazol-3-yl-4-{3-[(2-bromophenyl)-ethynyl]benzyl}-piperazine-1-carboxamide;
4-(3-Ethynyl-benzyl)-piperazine-1-carboxylic acid benzo[d]isoxazol-3-ylamide;
N-1,2-Benzisoxazol-3-yl-4-{3-[3-(dimethylamino)prop-1-yn-1-yl]benzyl}-piperazine-1-carboxamide;
N-1,2-Benzisoxazol-3-yl-4-[3-(cyclohexylethynyl)benzyl]-piperazine-1-carboxamide;
N-1,2-Benzisoxazol-3-yl-4-[3-(cyclopentylethynyl)benzyl]-piperazine-1-carboxamide;
N-1,2-Benzisoxazol-3-yl-4-{3-[(2-chlorophenyl)-ethynyl]benzyl}-piperazine-1-carboxamide;
N-1,2-Benzisoxazol-3-yl-4-{3-[(3-chlorophenyl)ethynyl]benzyl}-piperazine-1-carboxamide;
N-1,2-Benzisoxazol-3-yl-4-{3-[(4-chlorophenyl)ethynyl]benzyl}-piperazine-1-carboxamide;
N-1,2-Benzisoxazol-3-yl-4-{3-[(3,4-dichlorophenyl)ethynyl]-benzyl}-piperazine-1-carboxamide;
N-1,2-Benzisoxazol-3-yl-4-[3-(cyclopropylethynyl)benzyl]-piperazine-1-carboxamide;
N-1,2-Benzisoxazol-3-yl-4-[3-(thiophen-3-ylethynyl)benzyl]-piperazine-1-carboxamide;
4-{3-[(2-Chlorophenyl)ethynyl]benzyl}-N-pyrazin-2-ylpiperazine-1-carboxamide;
4-{3-[(2-Chlorophenyl)ethynyl]benzyl}-N-pyridazin-3-ylpiperazine-1-carboxamide;
4-{3-[(2-Chlorophenyl)ethynyl]benzyl}-N-(5-methylpyrazin-2-yl)piperazine-1-carboxamide;
N-1,2-Benzisoxazol-3-yl-4-{3-[(2,4-dichlorophenyl)-ethynyl]benzyl}-piperazine-1-carboxamide;
N-1,2-Benzisoxazol-3-yl-4-(3-{[2-(trifluoromethoxy)phenyl]-ethynyl}benzyl)-piperazine-1-carboxamide;
N-1,2-Benzisoxazol-3-yl-4-{3-[(3,5-dichlorophenyl)ethynyl]benzyl}-piperazine-1-carboxamide;
N-1,2-Benzisoxazol-3-yl-4-{3-[(2,5-dichlorophenyl)ethynyl]benzyl}-piperazine-1-carboxamide;
N-1,2-Benzisoxazol-3-yl-4-{3-[(2-cyanophenyl)ethynyl]benzyl}-piperazine-1-carboxamide;
N-1,2-Benzisoxazol-3-yl-4-[3-(naphthalen-1-ylethynyl)benzyl]piperazine-1-carboxamide;
Methyl 2-[(3-{[4-(1,2-benzisoxazol-3-ylcarbamoyl)piperazin-1-yl]methyl}phenyl)ethynyl]benzoate;
N-1,2-Benzisoxazol-3-yl-4-{3-[(3-cyanophenyl)ethynyl]benzyl}piperazine-1-carboxamide;
N-1,2-Benzisoxazol-3-yl-4-[3-(1,3-benzodioxol-5-ylethynyl)benzyl]-piperazine-1-carboxamide;
N-1,2-Benzisoxazol-3-yl-4-{3-[(2,3-dichlorophenyl)ethynyl]benzyl}-piperazine-1-carboxamide;
N-1,2-Benzisoxazol-3-yl-4-{3-[(2-cyano-3-fluorophenyl)ethynyl]-benzyl}piperazine-1-carboxamide;
N-1,2-Benzisoxazol-3-yl-4-(3-{[2-(cyanomethyl)phenyl]ethynyl}-benzyl)piperazine-1-carboxamide;
Methyl {2-[(3-{[4-(1,2-benzisoxazol-3-ylcarbamoyl)piperazin-1-yl]methyl}phenyl)ethynyl]phenyl}acetate;
4-[3-(2-o-Tolyl-ethyl)-benzyl]-piperazine-1-carboxylic acid benzo[d]isoxazol-3-ylamide;
4-[3-(Pyrimidin-2-yloxy)-benzyl]-piperazine-1-carboxylic acid benzo[d]isoxazol-3-ylamide;
N-1,2-Benzisoxazol-3-yl-4-[3-(pyridin-2-yloxy)benzyl]piperazine-1-carboxamide;
N-1,2-Benzisoxazol-3-yl-4-[3-(pyrazin-2-yloxy)benzyl]piperazine-1-carboxamide;
4-[3-(2-Cyano-benzyloxy)-benzyl]-piperazine-1-carboxylic acid benzo[d]isoxazol-3-ylamide;
N-1,2-Benzisoxazol-3-yl-4-[3-(benzyloxy)benzyl]piperazine-1-carboxamide;
4-(1H-Benzimidazol-6-ylmethyl)-N-1,2-benzisoxazol-3-ylpiperazine-1-carboxamide;
N-1,2-Benzisoxazol-3-yl-4-(1H-indazol-6-ylmethyl)piperazine-1-carboxamide;
N-1,2-Benzisoxazol-3-yl-4-[4-(methylsulfonyl)benzyl]piperazine-1-carboxamide;
N-1,2-Benzisoxazol-3-yl-4-[4-(trifluoromethoxy)benzyl]piperazine-1-carboxamide;
4-[3-(4-Chlorophenoxy)benzyl]-N-(6-methoxypyridazin-3-yl)piperazine-1-carboxamide;
N-1,2-Benzisoxazol-3-yl-4-[4-chloro-3-(trifluoromethoxy)benzyl]-piperazine-1-carboxamide;
N-1,2-Benzisoxazol-3-yl-4-[4-fluoro-3-(trifluoromethoxy)benzyl]-piperazine-1-carboxamide;
N-1,2-Benzisoxazol-3-yl-4-[3-chloro-4-(trifluoromethoxy)benzyl]-piperazine-1-carboxamide;
N-1,2-Benzisoxazol-3-yl-4-[3-fluoro-4-(trifluoromethoxy)benzyl]-piperazine-1-carboxamide;
N-1,2-Benzisoxazol-3-yl-4-{3-[4-(trifluoromethyl)phenoxy]benzyl-piperazine-1-carboxamide;
N-1,2-Benzisoxazol-3-yl-4-(3-phenoxybenzyl)piperazine-1-carboxamide;
N-1,2-Benzisoxazol-3-yl-4-(3,4-dichlorobenzyl)piperazine-1-carboxamide;
N-1,2-Benzisoxazol-3-yl-4-[4-(benzyloxy)benzyl]piperazine-1-carboxamide;
N-1,2-Benzisoxazol-3-yl-4-(1-benzothiophen-2-ylmethyl)piperazine-1-carboxamide;
N-1,2-Benzisoxazol-3-yl-4-[3-(quinolin-6-yloxy)benzyl]piperazine-1-carboxamide;
N-1,2-Benzisoxazol-3-yl-4-(4-bromo-3-fluorobenzyl)piperazine-1-carboxamide;
N-1,2-Benzisoxazol-3-yl-4-(1,3-benzodioxol-5-ylmethyl)piperazine-1-carboxamide;
N-1,2-Benzisoxazol-3-yl-4-(quinolin-3-ylmethyl)piperazine-1-carboxamide;
N-1,2-Benzisoxazol-3-yl-4-(1H-indol-5-ylmethyl)piperazine-1-carboxamide;
N-1,2-Benzisoxazol-3-yl-4-[3-(naphthalen-2-yloxy)benzyl]piperazine-1-carboxamide;
N-1,2-Benzisoxazol-3-yl-4-(4-bromobenzyl)piperazine-1-carboxamide;
N-1,2-Benzisoxazol-3-yl-4-(3,4-dibromobenzyl)piperazine-1-carboxamide;
N-1,2-Benzisoxazol-3-yl-4-[3-(2-chlorophenoxy)benzyl]piperazine-1-carboxamide;
4-Naphthalen-2-ylmethyl-piperazine-1-carboxylic acid benzo[d]isoxazol-3-ylamide;
4-Quinolin-2-ylmethyl-piperazine-1-carboxylic acid benzo[d]isoxazol-3-ylamide;
4-[3-(4-Cyano-phenoxy)-benzyl]-piperazine-1-carboxylic acid benzo[d]isoxazol-3-ylamide;
4-Benzofuran-2-ylmethyl-piperazine-1-carboxylic acid benzo[d]isoxazol-3-ylamide;
N-1,2-Benzisoxazol-3-yl-4-[3-(3-chlorophenoxy)benzyl]piperazine-1-carboxamide;
N-1,2-Benzisoxazol-3-yl-4-{3-[4-cyano-3-(trifluoromethyl)phenoxy]benzyl}piperazine-1-carboxamide;
N-1,2-Benzisoxazol-3-yl-4-[3-(3-cyanophenoxy)benzyl]piperazine-1-carboxamide;
N-1,2-Benzisoxazol-3-yl-4-(3-{4-[(trifluoromethyl)sulfanyl]phenoxy}-benzyl)piperazine-1-carboxamide;
N-1,2-Benzisoxazol-3-yl-4-{3-[(2,2-difluoro-1,3-benzodioxol-5-yl)oxy]benzyl}piperazine-1-carboxamide;
N-1,2-Benzisoxazol-3-yl-4-(3-{4-[(trifluoromethyl)sulfonyl]phenoxy}-benzyl)piperazine-1-carboxamide;
N-1,2-Benzisoxazol-3-yl-4-{[3-(phenylethynyl)phenyl]methyl}piperazine-1-carboxamide;
N-Isoxazol-3-yl-4-{3-[4-(trifluoromethoxy)phenoxy]benzyl}piperazine-1-carboxamide;
4-[4-(Benzyloxy)benzyl]-N-isoxazol-3-ylpiperazine-1-carboxamide;
4-[3-(3-Chlorophenoxy)benzyl]-N-isoxazol-3-ylpiperazine-1-carboxamide;
N-isoxazol-3-yl-4-{3-[4-(2,2,2-trifluoroethoxy)phenoxy]benzyl}piperazine-1-carboxamide;
4-(1-Benzofuran-2-ylmethyl)-N-isoxazol-3-ylpiperazine-1-carboxamide;
4-[3-(3-Cyanophenoxy)benzyl]-N-isoxazol-3-ylpiperazine-1-carboxamide;
4-[3-(2-Chlorophenoxy)benzyl]-N-isoxazol-3-ylpiperazine-1-carboxamide;
4-{3-[(2,2-Difluoro-1,3-benzodioxol-5-yl)oxy]benzyl}-N-isoxazol-3-ylpiperazine-1-carboxamide;
4-(1-Benzothiophen-2-ylmethyl)-N-isoxazol-3-ylpiperazine-1-carboxamide;
4-(1,3-Benzodioxol-5-ylmethyl)-N-isoxazol-3-ylpiperazine-1-carboxamide;
N-Isoxazol-3-yl-4-(naphthalen-2-ylmethyl)piperazine-1-carboxamide;
4-[3-(4-Bromophenoxy)benzyl]-N-isoxazol-3-ylpiperazine-1-carboxamide;
4-Quinolin-2-ylmethyl-piperazine-1-carboxylic acid isoxazol-3-ylamide;
4-Quinolin-3-ylmethyl-piperazine-1-carboxylic acid isoxazol-3-ylamide;
4-(4-Bromo-benzyl)-piperazine-1-carboxylic acid isoxazol-3-ylamide;
4-(1H-Indol-6-ylmethyl)-piperazine-1-carboxylic acid isoxazol-3-ylamide;
4-[3-(Naphthalen-2-yloxy)-benzyl]-piperazine-1-carboxylic acid isoxazol-3-ylamide;
4-(4-Bromo-3-fluoro-benzyl)-piperazine-1-carboxylic acid isoxazol-3-ylamide;
4-[3-(4-Cyano-phenoxy)-benzyl]-piperazine-1-carboxylic acid isoxazol-3-ylamide;
4-[3-(3,4-Difluorophenoxy)benzyl]-N-isoxazol-3-ylpiperazine-1-carboxamide;
4-(3,4-Dibromobenzyl)-N-isoxazol-3-ylpiperazine-1-carboxamide;
N-Isoxazol-3-yl-4-(3-{4-[(trifluoromethyl)suIfanyl]phenoxy}benzyl)-piperazine-1-carboxamide;
4-{3-[4-Fluoro-3-(trifluoromethyl)phenoxy]benzyl}-N-isoxazol-3-ylpiperazine-1-carboxamide;
4-[3-(3-Bromophenoxy)benzyl]-N-isoxazol-3-ylpiperazine-1-carboxamide;
N-Isoxazol-3-yl-4-(3-{4-[(trifluoromethyl)sulfonyl]phenoxy}benzyl)-piperazine-1-carboxamide;
N-Isoxazol-3-yl-4-{3-[3-(trifluoromethoxy)phenoxy]benzyl}piperazine-1-carboxamide;
4-(3,4-Dichlorobenzyl)-N-isoxazol-3-ylpiperazine-1-carboxamide;
N-Isoxazol-3-yl-4-{3-[4-(trifluoromethyl)phenoxy]benzyl}piperazine-1-carboxamide;
N-Isoxazol-3-yl-4-[3-(quinolin-6-yloxy)benzyl]piperazine-1-carboxamide;
4-{3-[4-Cyano-3-(trifluoromethyl)phenoxy]benzyl}-N-isoxazol-3-ylpiperazine-1-carboxamide;
4-[3-(4-Chlorophenoxy)benzyl]-N-(5-methylisoxazol-3-yl)piperazine-1-carboxamide;
4-(Quinolin-3-ylmethyl)-N-1H-tetrazol-5-ylpiperazine-1-carboxamide;
4-[3-(Naphthalen-2-yloxy)benzyl]-N-1H-tetrazol-5-ylpiperazine-1-carboxamide;
4-(3,4-Dibromobenzyl)-N-1H-tetrazol-5-ylpiperazine-1-carboxamide;
4-(4-Bromo-3-fluorobenzyl)-N-1H-tetrazol-5-ylpiperazine-1-carboxamide;
4-[3-(3,4-Difluorophenoxy)benzyl]-N-1H-tetrazol-5-ylpiperazine-1-carboxamide;
4-{3-[4-Cyano-3-(trifluoromethyl)phenoxy]benzyl}-N-1H-tetrazol-5-ylpiperazine-1-carboxamide;
N-1H-Tetrazol-5-yl-4-{3-[4-(trifluoromethyl)phenoxy]benzyl}piperazine-1-carboxamide;
N-1H-Tetrazol-5-yl-4-(3-{4-[(trifluoromethyl)sulfanyl]phenoxy}benzyl)-piperazine-1-carboxamide;
N-1H-Tetrazol-5-yl-4-{3-[3-(trifluoromethoxy)phenoxy]benzyl}piperazine-1-carboxamide;
4-[3-(3,4-Dichlorophenoxy)benzyl]-N-1H-tetrazol-5-ylpiperazine-1-carboxamide;
4-(Quinolin-2-ylmethyl)-N-1H-tetrazol-5-ylpiperazine-1-carboxamide;
4-(Naphthalen-2-ylmethyl)-N-1H-tetrazol-5-ylpiperazine-1-carboxamide;
4-(4-Bromo-benzyl)-piperazine-1-carboxylic acid (2H-tetrazol-5-yl)-amide;
4-(1H-Indol-6-ylmethyl)-piperazine-1-carboxylic acid (2H-tetrazol-5-yl)-amide;
4-(3-Benzyloxy-benzyl)-piperazine-1-carboxylic acid (2H-tetrazol-5-yl)-amide;
4-Benzo[1,3]dioxol-5-ylmethyl-piperazine-1-carboxylic acid (2H-tetrazol-5-yl)-amide;
4-(3-Phenoxy-benzyl)-piperazine-1-carboxylic acid (2H-tetrazol-5-yl)-amide;
4-(3,4-Dichloro-benzyl)-piperazine-1-carboxylic acid (2H-tetrazol-5-yl)-amide;
4-Benzo[b]thiophen-2-ylmethyl-piperazine-1-carboxylic acid (2H-tetrazol-5-yl)-amide;
4-[3-(3-Cyano-phenoxy)-benzyl]-piperazine-1-carboxylic acid (2H-tetrazol-5-yl)-amide;
4-{3-[4-Fluoro-3-(trifluoromethyl)phenoxy]benzyl}-N-2H-tetrazol-5-yl piperazine-1-carboxamide;
N-2H-Tetrazol-5-yl-4-{3-[3-(trifluoromethyl)phenoxy]benzyl}piperazine-1-carboxamide;
4-[3-(4-Cyanophenoxy)benzyl]-N-2H-tetrazol-5-ylpiperazine-1-carboxamide;
N-2H-Tetrazol-5-yl-4-{3-[4-(2,2,2-trifluoroethoxy)phenoxy]benzyl}-piperazine-1-carboxamide;
4-{3-[(2,2-Difluoro-1,3-benzodioxol-5-yl)oxy]benzyl}-N-2H-tetrazol-5-ylpiperazine-1-carboxamide;
4-[3-(2-Chlorophenoxy)benzyl]-N-2H-tetrazol-5-ylpiperazine-1-carboxamide;
4-[3-(4-Chloro-phenoxy)-benzyl]-piperazine-1-carboxylic acid (1,5-dimethyl-1H-pyrazol-3-yl)-amide;
4-[3-(4-Chloro-phenoxy)-benzyl]-piperazine-1-carboxylic acid (4-bromo-1-methyl-1H-pyrazol-3-yl)-amide;
4-[3-(4-Chloro-phenoxy)-benzyl]-piperazine-1-carboxylic acid (2-ethyl-2H-pyrazol-3-yl)-amide;
4-[3-(4-Chlorophenoxy)benzyl]-N-(5-methyl-1H-pyrazol-3-yl)piperazine-1-carboxamide;
4-(3,4-Dibromobenzyl)-N-pyridazin-3-ylpiperazine-1-carboxamide;
4-[(2,2-Difluoro-1,3-benzodioxol-5-yl)methyl]-N-pyridazin-3-ylpiperazine-1-carboxamide;
N-Pyridazin-3-yl-4-(quinolin-3-ylmethyl)piperazine-1-carboxamide;
N-Pyridazin-3-yl-4-(quinolin-2-ylmethyl)piperazine-1-carboxamide;
4-(3,4-Dichlorobenzyl)-N-pyridazin-3-ylpiperazine-1-carboxamide;
4-(Naphthalen-2-ylmethyl)-N-pyridazin-3-ylpiperazine-1-carboxamide;
4-(1H-Indol-5-ylmethyl)-N-pyridazin-3-ylpiperazine-1-carboxamide;
N-2,1,3-Benzothiadiazol-4-yl-4-{[3-(phenylethynyl)phenyl]methyl}-piperazine-1-carboxamide;
N-2,1,3-Benzoxadiazol-4-yl-4-{[3-(phenylethynyl)phenyl]methyl}-piperazine-1-carboxamide;
4-[3-(3-Chloro-4-trifluoromethyl-phenoxy)-benzyl]-piperazine-1-carboxylic acid benzo[d]isoxazol-3-ylamide;
4-[3-(4-Chloro-3-trifluoromethyl-phenoxy)-benzyl]-piperazine-1-carboxylic acid benzo[d]isoxazol-3-ylamide;
4-[3-(4-Chloro-3-fluoro-phenoxy)-benzyl]-piperazine-1-carboxylic acid benzo[d]isoxazol-3-ylamide;
4-[3-(3-Chloro-4-fluoro-phenoxy)-benzyl]-piperazine-1-carboxylic acid benzo[d]isoxazol-3-ylamide;
4-[3-(4-Fluoro-phenoxy)-benzyl]-piperazine-1-carboxylic acid benzo[d]isoxazol-3-ylamide;
4-[3-(4-Butyl-phenoxy)-benzyl]-piperazine-1-carboxylic acid benzo[d]isoxazol-3-ylamide;
4-[(2,2-Difluoro-1,3-benzodioxol-5-yl)methyl]-N-pyrazin-2-ylpiperazine-1-carboxamide;
4-(1,3-Benzodioxol-5-ylmethyl)-N-pyrazin-2-ylpiperazine-1-carboxamide;
4-(4-Bromobenzyl)-N-pyrazin-2-ylpiperazine-1-carboxamide;
4-(Naphthalen-2-ylmethyl)-N-pyrazin-2-ylpiperazine-1-carboxamide;
N-Pyrazin-2-yl-4-{3-[4-(2,2,2-trifluoroethoxy)phenoxy]benzyl}-piperazine-1-carboxamide;
N-Pyrazin-2-yl-4-{3-[4-(trifluoromethyl)phenoxy]benzyl}piperazine-1-carboxamide;
4-(1H-Indol-5-ylmethyl)-N-pyrazin-2-ylpiperazine-1-carboxamide;
4-(3,4-Dibromobenzyl)-N-pyrazin-2-ylpiperazine-1-carboxamide;
4-(1-Benzothiophen-2-ylmethyl)-N-pyrazin-2-ylpiperazine-1-carboxamide;
4-[4-(Benzyloxy)benzyl]-N-pyrazin-2-ylpiperazine-1-carboxamide;
4-(3,4-Dichlorobenzyl)-N-pyrazin-2-ylpiperazine-1-carboxamide;
4-[3-(4-Bromophenoxy)benzyl]-N-pyrazin-2-ylpiperazine-1-carboxamide;
4-(4-Bromo-3-fluorobenzyl)-N-pyrazin-2-ylpiperazine-1-carboxamide;
4-[3-(Benzyloxy)benzyl]-N-pyrazin-2-ylpiperazine-1-carboxamide;
N-Pyrazin-2-yl-4-(quinolin-3-ylmethyl)piperazine-1-carboxamide;
4-[3-(3-Chlorophenoxy)benzyl]-N-pyrazin-2-ylpiperazine-1-carboxamide;
N-Pyrazin-2-yl-4-(3-{4-[(trifluoromethyl)sulfonyl]phenoxy}benzyl)-piperazine-1-carboxamide;
4-(3-Phenoxy-benzyl)-piperazine-1-carboxylic acid pyrazin-2-ylamide;
4-[3-(Naphthalen-2-yloxy)-benzyl]-piperazine-1-carboxylic acid pyrazin-2-ylamide;
4-[3-(4-Cyano-phenoxy)-benzyl]-piperazine-1-carboxylic acid pyrazin-2-ylamide;
4-Benzofuran-2-ylmethyl-piperazine-1-carboxylic acid pyrazin-2-ylamide;
4-[3-(3,4-Difluorophenoxy)benzyl]-N-pyrazin-2-ylpiperazine-1-carboxamide;
N-Pyrazin-2-yl-4-[3-(quinolin-6-yloxy)benzyl]piperazine-1-carboxamide;
N-Pyrazin-2-yl-4-{3-[4-(trifluoromethoxy)phenoxy]benzyl}piperazine-1-carboxamide;
N-Pyrazin-2-yl-4-(3-{4-[(trifluoromethyl)sulfanyl]phenoxy}benzyl)-piperazine-1-carboxamide;
4-[3-(3-Cyanophenoxy)benzyl]-N-pyrazin-2-ylpiperazine-1-carboxamide;
4-{3-[4-Cyano-3-(trifluoromethyl)phenoxy]benzyl}-N-pyrazin-2-ylpiperazine-1-carboxamide;
4-{3-[(2,2-Difluoro-1,3-benzodioxol-5-yl)oxy]benzyl}-N-pyrazin-2-ylpiperazine-1-carboxamide;
4-[3-(2-Chlorophenoxy)benzyl]-N-pyrazin-2-ylpiperazine-1-carboxamide;
N-Pyrazin-2-yl-4-(quinolin-2-ylmethyl)piperazine-1-carboxamide;
4-[3-(3-Bromophenoxy)benzyl]-N-pyrazin-2-ylpiperazine-1-carboxamide;
4-{3-[4-Fluoro-3-(trifluoromethyl)phenoxy]benzyl}-N-pyrazin-2-ylpiperazine-1-carboxamide;
N-Pyrazin-2-yl-4-{3-[3-(trifluoromethoxy)phenoxy]benzyl}piperazine-1-carboxamide;
4-[3-(4-Chlorophenoxy)benzyl]-N-(3-chloropyrazin-2-yl)piperazine-1-carboxamide;
4-[3-(4-Chlorophenoxy)benzyl]-N-(5-phenyl-1H-pyrazol-3-yl)piperazine-1-carboxamide;
4-[3-(4-Chloro-phenoxy)-benzyl]-piperazine-1-carboxylic acid (6-fluoro-benzo[d]isoxazol-3-yl)-amide; and
4-[3-(4-Chloro-phenoxy)-benzyl]-piperazine-1-carboxylic acid pyridazin-3-ylamide;
and pharmaceutically acceptable salts thereof.

40. A method according to claim 38, wherein the disease, disorder, or medical condition is selected from the group consisting of: anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, and neuroinflammation.

41. A method according to claim 38, wherein the disease, disorder, or medical condition is pain or inflammation.

42. A method according to claim 38, wherein the disease, disorder, or medical condition is anxiety, a sleep disorder, an eating disorder, or a movement disorder.

43. A method according to claim 38, wherein the disease, disorder, or medical condition is multiple sclerosis.

44. A method according to claim 38, wherein the disease, disorder, or medical condition is energy metabolism or bone homeostasis.

45. A method of treating a subject suffering from or diagnosed with a disease, disorder, or medical condition mediated by FAAH activity, comprising administering to the subject in need of such treatment an effective amount of at least one active selected from compounds of Formula (Ia): 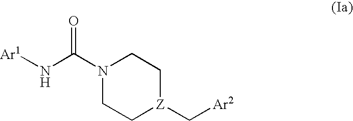
wherein:
Ar¹ is a benzo[d]isoxazol-3-yl, 6-fluorobenzo[d]isoxazol-3-yl, 3-phenyl-[1,2,4]thiadiazol-5-yl, 1H-tetrazol-5-yl, benzo[1,2,5]thiadiazol-4-yl, benzo[1,2,5]oxadiazol-4-yl, thiophen-2-yl, thiophen-3-yl, 6-chloro-pyridazin-3-yl, pyrazin-2-yl, isoxazol-3-yl, 1H-benzotriazol-5-yl, [1,5]naphthyridin-2-yl, quinolin-2-yl, benzothiazol-6-yl, quinolin-5-yl, or 1H-pyrazol-3-yl group;
Z is —N— or >CH; and
Ar² is:
(i) phenyl or 3-phenoxyphenyl substituted with one or two R^{a} moieties;
where each R^{a} moiety is independently —C₁₋₄alkyl, —OC₁₋₄alkyl, halo, —CF₃, —OCF₃, —OCH₂CF₃, —SCF₃, —S(O)₀₋₂C₁₋₄alkyl, —OSO₂C₁₋₄alkyl, —CO₂C₁₋₄alkyl, —CO₂H, —COC₁₋₄alkyl, —N(R^{b})R^{c}, —SO₂NR^{b}R^{c}, —NR^{b}SO₂R^{c}, —C(O)NR^{b}R^{c}, —NO₂, or —CN;
where R^{b} and R^{c} are each independently —H or —C₁₋₄alkyl; or
(ii) benzo[1,3]dioxol-5-yl, 2,2-difluoro-benzo[1,3]dioxol-5-yl, or naphthyl;
where when Ar¹ is 6-chloro-pyridazin-3-yl, isoxazol-3-yl, or 1H-pyrazol-3-yl, then Ar² is not benzo[1,3]dioxol-5-yl or 2,2-difluoro-benzo[1,3]dioxol-5-yl; and
pharmaceutically acceptable salts, pharmaceutically acceptable prodrugs, and
pharmaceutically active metabolites of said compounds of Formula (Ia).

46. A method according to claim 45, wherein said active agent is selected from the group consisting of:
4-(2,2-Difluoro-benzo[1,3]dioxol-5-ylmethyl)-piperazine-1-carboxylic acid benzo[d]isoxazol-3-ylamide;
4-(2,2-Difluoro-benzo[1,3]dioxol-5-ylmethyl)-piperazine-1-carboxylic acid (3-phenyl-[1,2,4]thiadiazol-5-yl)-amide;
4-(2,2-Difluoro-benzo[1,3]dioxol-5-ylmethyl)-piperazine-1-carboxylic acid (1H-tetrazol-5-yl)-amide;
4-(2,2-Difluoro-benzo[1,3]dioxol-5-ylmethyl)-piperazine-1-carboxylic acid benzo[1,2,5]thiadiazol-4-ylamide;
4-(2,2-Difluoro-benzo[1,3]dioxol-5-ylmethyl)-piperazine-1-carboxylic acid benzo[1,2,5]oxadiazol-4-ylamide;
4-(2,2-Difluoro-benzo[1,3]dioxol-5-ylmethyl)-piperazine-1-carboxylic acid (3H-benzotriazol-5-yl)-amide;
4-(2,2-Difluoro-benzo[1,3]dioxol-5-ylmethyl)-piperazine-1-carboxylic acid thiophen-2-ylamide;
4-(2,2-Difluoro-benzo[1,3]dioxol-5-ylmethyl)-piperazine-1-carboxylic acid thiophen-3-ylamide;
4-Naphthalen-2-ylmethyl-piperidine-1-carboxylic acid (6-chloro-pyridazin-3-yl)-amide;
4-Naphthalen-2-ylmethyl-piperidine-1-carboxylic acid pyrazin-2-ylamide;
4-Naphthalen-2-ylmethyl-piperidine-1-carboxylic acid isoxazol-3-ylamide;
4-Naphthalen-2-ylmethyl-piperidine-1-carboxylic acid (3-phenyl-[1,2,4]thiadiazol-5-yl)-amide;
4-Naphthalen-2-ylmethyl-piperidine-1-carboxylic acid (1H-tetrazol-5-yl)-amide;
4-Naphthalen-2-ylmethyl-piperidine-1-carboxylic acid (2H-pyrazol-3-yl)-amide;
4-Naphthalen-2-ylmethyl-piperidine-1-carboxylic acid benzo[1,2,5]oxadiazol-4-ylamide;
4-Naphthalen-2-ylmethyl-piperidine-1-carboxylic acid (1H-benzotriazol-5-yl)-amide;
4-Naphthalen-2-ylmethyl-piperidine-1-carboxylic acid [1,5]naphthyridin-2-ylamide;
4-Naphthalen-2-ylmethyl-piperidine-1-carboxylic acid quinolin-2-ylamide;
4-Naphthalen-2-ylmethyl-piperidine-1-carboxylic acid benzothiazol-6-ylamide;
4-Naphthalen-2-ylmethyl-piperidine-1-carboxylic acid quinolin-5-ylamide;
4-Naphthalen-2-ylmethyl-piperidine-1-carboxylic acid benzo[d]isoxazol-3-ylamide;
4-(4-Fluoro-benzyl)-piperidine-1-carboxylic acid benzo[d]isoxazol-3-ylamide;
4-(4-Fluoro-benzyl)-piperidine-1-carboxylic acid (6-chloro-pyridazin-3-yl)-amide;
4-(4-Fluoro-benzyl)-piperidine-1-carboxylic acid isoxazol-3-ylamide;
4-(3-Trifluoromethyl-benzyl)-piperidine-1-carboxylic acid (6-chloro-pyridazin-3-yl)-amide;
4-(3-Trifluoromethyl-benzyl)-piperidine-1-carboxylic acid isoxazol-3-ylamide;
4-(3-Trifluoromethyl-benzyl)-piperidine-1-carboxylic acid benzo[d]isoxazol-3-ylamide;
4-[3-(4-Fluoro-3-trifluoromethyl-phenoxy)-benzyl]-piperazine-1-carboxylic acid benzo[d]isoxazol-3-ylamide;
4-[3-(3-Trifluoromethoxy-phenoxy)-benzyl]-piperazine-1-carboxylic acid benzo[d]isoxazol-3-ylamide;
4-[3-(4-Trifluoromethoxy-phenoxy)-benzyl]-piperazine-1-carboxylic acid benzo[d]isoxazol-3-ylamide;
4-[3-(3-Bromo-phenoxy)-benzyl]-piperazine-1-carboxylic acid benzo[d]isoxazol-3-ylamide;
4-[3-(4-Bromo-phenoxy)-benzyl]-piperazine-1-carboxylic acid benzo[d]isoxazol-3-ylamide;
4-[3-(3,4-Difluoro-phenoxy)-benzyl]-piperazine-1-carboxylic acid benzo[d]isoxazol-3-ylamide;
4-[3-(3,5-Difluoro-phenoxy)-benzyl]-piperazine-1-carboxylic acid benzo[d]isoxazol-3-ylamide;
4-{3-[4-(2,2,2-Trifluoro-ethoxy)-phenoxy]-benzyl}-piperazine-1-carboxylic acid benzo[d]isoxazol-3-ylamide;
4-[3-(4-Chloro-phenoxy)-benzyl]-piperazine-1-carboxylic acid benzo[d]isoxazol-3-ylamide;
4-[3-(4-Chloro-phenoxy)-benzyl]-piperazine-1-carboxylic acid benzo[1,2,5]thiadiazol-4-ylamide;
4-[3-(3,4-Dichloro-phenoxy)-benzyl]-piperazine-1-carboxylic acid benzo[1,2,5]thiadiazol-4-ylamide;
4-[3-(3,5-Dichloro-phenoxy)-benzyl]-piperazine-1-carboxylic acid benzo[1,2,5]thiadiazol-4-ylamide;
4-[3-(3-Trifluoromethyl-phenoxy)-benzyl]-piperazine-1-carboxylic acid benzo[1,2,5]thiadiazol-4-ylamide;
4-(3-Trifluoromethoxy-benzyl)-piperazine-1-carboxylic acid benzo[1,2,5]thiadiazol-4-ylamide;
4-[3-(4-Chloro-phenoxy)-benzyl]-piperazine-1-carboxylic acid (6-chloro-pyridazin-3-yl)-amide;
4-[3-(3,4-Dichloro-phenoxy)-benzyl]-piperazine-1-carboxylic acid (6-chloro-pyridazin-3-yl)-amide;
4-[3-(3-Trifluoromethyl-phenoxy)-benzyl]-piperazine-1-carboxylic acid (6-chloro-pyridazin-3-yl)-amide;
4-(4-Fluoro-3-phenoxy-benzyl)-piperazine-1-carboxylic acid (6-chloro-pyridazin-3-yl)-amide;
4-[3-(4-Chloro-phenoxy)-benzyl]-piperazine-1-carboxylic acid isoxazol-3-ylamide;
4-[3-(3,4-Dichloro-phenoxy)-benzyl]-piperazine-1-carboxylic acid isoxazol-3-ylamide;
4-[3-(3-Trifluoromethyl-phenoxy)-benzyl]-piperazine-1-carboxylic acid isoxazol-3-ylamide;
4-(4-Fluoro-3-phenoxy-benzyl)-piperazine-1-carboxylic acid isoxazol-3-ylamide;
4-[3-(3,4-Dichloro-phenoxy)-benzyl]-piperazine-1-carboxylic acid benzo[d]isoxazol-3-ylamide;
4-[3-(3-Trifluoromethyl-phenoxy)-benzyl]-piperazine-1-carboxylic acid benzo[d]isoxazol-3-ylamide;
4-(3-Trifluoromethoxy-benzyl)-piperazine-1-carboxylic acid benzo[d]isoxazol-3-ylamide;
4-[3-(4-Chloro-phenoxy)-benzyl]-piperazine-1-carboxylic acid (1H-pyrazol-3-yl)-amide;
4-[3-(3,4-Dichloro-phenoxy)-benzyl]-piperazine-1-carboxylic acid (1H-pyrazol-3-yl)-amide;
4-[3-(3-Trifluoromethyl-phenoxy)-benzyl]-piperazine-1-carboxylic acid (1H-pyrazol-3-yl)-amide;
4-(4-Fluoro-3-phenoxy-benzyl)-piperazine-1-carboxylic acid (1H-pyrazol-3-yl)-amide;
4-[3-(4-Chloro-phenoxy)-benzyl]-piperazine-1-carboxylic acid (1H-tetrazol-5-yl)-amide;
4-[3-(4-Chloro-phenoxy)-benzyl]-piperazine-1-carboxylic acid pyrazin-2-ylamide;
4-[3-(3,4-Dichloro-phenoxy)-benzyl]-piperazine-1-carboxylic acid pyrazin-2-ylamide;
4-[3-(3-Trifluoromethyl-phenoxy)-benzyl]-piperazine-1-carboxylic acid pyrazin-2-ylamide; and
4-(3-Trifluoromethoxy-benzyl)-piperazine-1-carboxylic acid pyrazin-2-ylamide;
and pharmaceutically acceptable salts thereof.

47. A method according to claim 45, wherein the disease, disorder, or medical condition is selected from the group consisting of: anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, and neuroinflammation.

48. A method according to claim 45, wherein the disease, disorder, or medical condition is pain or inflammation.

49. A method according to claim 45, wherein the disease, disorder, or medical condition is anxiety, a sleep disorder, an eating disorder, or a movement disorder.

50. A method according to claim 45, wherein the disease, disorder, or medical condition is multiple sclerosis.

51. A method according to claim 45, wherein the disease, disorder, or medical condition is energy metabolism or bone homeostasis.
